(19) United States
(12) Patent Application Publication
(10) Pub. No.: US 2021/0030802 A1
(43) Pub. Date: Feb. 4, 2021

(54) GLUCOCORTICOID-RESISTANT LEUKOCYTES AND THEIR USE IN THE TREATMENT OF CANCERS AND VIRUSES

(71) Applicant: CONSTANT BIOTECHNOLOGY, LLC, Covington, KY (US)

(72) Inventor: Brian R. CLARK, Loveland, OH (US)

(73) Assignee: CONSTANT BIOTECHNOLOGY, LLC, Covington, KY (US)

(21) Appl. No.: 17/044,161

(22) PCT Filed: Apr. 5, 2019
(86) PCT No.: PCT/US2019/026081

§ 371 Date: Sep. 30, 2020

Related U.S. Application Data
(60) Provisional application No. 62/654,332, filed on Apr. 7, 2018.

(51) Int. Cl. A61K 35/17; A61K 31/235; A61K 38/17; A61K 31/573; A61P 35/00

(52) U.S. Cl. CPC A61K 35/17; A61K 31/235; A61P 35/00; A61K 31/573; A61K 38/1774

(57) ABSTRACT

A composition including genetically modified leukocytes is provided, where the genetically modified leukocytes contains a gene or expresses a protein that confers reversible resistance to glucocorticoids. In various aspects, the gene that confers resistance to glucocorticoids encodes 11-beta-dehydrogenase. Administering such genetically modified leukocytes provides leukocyte functions in treating one or more auto-immune, inflammatory, infectious or cancerous diseases or disorders, where the leukocytes are resistant to the effects of glucocorticoids such as alterations of numerous gene transcriptions in the leukocytes. Methods of reversing the glucocorticoid resistance in such genetically modified leukocytes are also provided by administering inhibitors of 11-beta-hydroxysteroid dehydrogenase. Methods of modifying the growth of these genetically modified leukocytes, or identification of candidate inhibitors of glucocorticoid resistance based on these genetically modified leukocytes, are also provided.

### CROSS-REFERENCE TO RELATED APPLICATIONS

This application includes a claim of priority under 35 U.S.C. § 119(e) to U.S. provisional patent application No. 62/654,332, filed Apr. 7, 2018, the entirety of which is hereby incorporated by reference.

### FIELD OF INVENTION

This invention relates to adoptive cellular therapy, and more specifically to genetically modified leukocytes that are resistant to immunosuppressive glucocorticoids.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Glucocorticoids are used pharmaceutically in medicine for a number of indications including: to suppress immune reactions, to reduce inflammation and swelling including cerebral edema and to promote lung maturation in premature babies. Excessive circulating glucocorticoids can also arise in Cushing's syndrome and as a result of certain tumors. Glucocorticoids have inhibitory effects on a broad range of immune responses. Most of the anti-inflammatory and immunosuppressive actions of glucocorticoids are attributable either directly or indirectly to the transcriptional effects of glucocorticoid receptor (abbreviated “GR”) agonism which alters transcription of numerous genes in leukocytes.

Glucocorticoids such as cortisol can be found in the plasma bound to transcortin (encoded by the gene SERPINA6) or serum albumin.

Normal physiology requires certain cells and tissues to be relatively “resistant” to the effects of glucocorticoids (“steroids”) or act as a barrier to the diffusion of steroids. These include: (a) cells in specific regions of the kidney that must eliminate the action of glucocorticoids (e.g., cortisol) in order to respond selectively to the structurally related mineralocorticoids since the mineralocorticoid receptor has similar affinities for the glucocorticoid cortisol (or corticosterone) and the mineralocorticoid aldosterone, and (b) the placenta, which can act as a barrier to the diffusion of (a finite level of) maternal glucocorticoids in during normal fetal development.

The physiological mechanism used to achieve steroid resistance is the expression of enzymes which utilize glucocorticoids as substrates. These enzymes generate a product steroid which has a lower affinity for the GR (gene name abbreviation NR3C1). In other words, in certain tissues, enzymes degrade glucocorticoids into less active (or inactive) forms.

U.S. Pat. No. 9,217,026 describes targeted cleavage of both copies of the glucocorticoid receptor (GR) gene in the genome of the cell to render cells resistant to glucocorticoids. Disruption of the GR allele in leukocytes in this manner is time-consuming and requires extensive selection and genomic analysis of tested cells. Moreover, because both copies of the GR allele are disrupted, GR-disrupted cells may be unresponsive to the administration of glucocorticoids that could otherwise be used to control adverse and deleterious immune responses. Menger L. et al., describe the use of the TALEN system to cleave the GR gene in an attempt to confer steroid unresponsiveness on T cells. Barrett A J, et al. describe two other routes to potentially confer resistance to steroids in adoptively transferred T cells, i.e., engineering T cells to overexpress 11β-hydroxysteroid dehydrogenases type 2 (11β-HSD2), which converts active GC, cortisol, to inactive cortisone, thereby inducing steroid resistance, as well as blocking Nfil3 or its signaling downstream of the GR to reduce glucocorticoid-induced apoptosis in T cells.

Other studies have looked at the transfection of a certain number of cell types with glucocorticoid-degrading enzymes, e.g., 11-beta-dehydrogenases. Tested glucocorticoid-degrading enzymes included hydroxysteroid 11-beta dehydrogenase 2 (HSD11B2, also known as corticosteroid 11-beta-dehydrogenase isozyme 2), hydroxysteroid 11-beta-dehydrogenase 1-like protein (HSD11B1L), and hydroxysteroid 11-beta dehydrogenase 1 (HSD1B1, also known as corticosteroid 11-beta-dehydrogenase isozyme 1). In the application, the gene or gene product of HSD11B1 is often referred to as “HSD1,” the gene or gene product of HSD11B2 as “HSD2,” and hydroxysteroid dehydrogenase activity abbreviated to “HSD”.

For example, Chinese Hamster Ovary (CHO) cells were transfected with HSD11B1 and HSD11B2 and their ability to convert 11-hydroxyl and keto forms of a glucocorticoid (cortisol and cortisone, respectively) was assayed. HEK-293 cells (from human embryonic kidney) were transduced with 11 beta-hydroxysteroid dehydrogenase type 1 genes from human, mouse, rat, hamster, guinea-pig and dog, where cell lysates were assayed for 11β-Hydroxysteroid dehydrogenase type 1 activity on cortisole and dehydrocorticosterone. Genes for human and mouse 11β-hydroxysteroid dehydrogenases (11-beta HSD) were transfected into Pichia Pastoris, where 11-beta HSD activity was assayed with potential inhibitors of 11-beta HSD. The gene for human HSD11B1 was co-transfected into HEK-293 cells (from human embryonic kidney) and HepG2/C3A cells (human hepatocellular carcinoma) along with a glucocorticoid-responsive luciferase reporter gene system to study 11β-hydroxysteroid dehydrogenase activity. The genes for human HSD11B1 and HSD11B2 were transfected into HEK-293 cells and cell lysates assayed for HSD activity. The genes for human HSD11B1 and HSD11B2 were transfected into HEK-293 cells and cell lysates assayed for HSD activity. The genes for human HSD11B1, HSD11B2 and variant-b of human HSD11B1L (“11-beta-HSD3” therein) were transfected into HEK-293 cells and intact cells assayed for HSD activity. In another study, HEK-293 cells were transfected with plasmids for HSD11B1 and HSD11B2. The HSD activity was assayed in cell lysates and glucocorticoid responsiveness assessed using a GR-reporter gene construct in intact cells. No steroid dehydrogenase activity (determined by no detectable conversion of cortisol into cortisone) was detected in stimulated mouse lymphocytes.

However, none of the aforementioned work has attempted transfecting or transducing HSD into leukocytes. This is due to various reasons, and among them, it remains challenging to develop reversible glucocorticoid resistance in leukocytes, or a steroid resistance that can be readily overcome.

Therefore, it is an objective of the present invention to provide genetically modified leukocytes with controlled resistance to glucocorticoid.

It is another objective of the present invention to provide a method of genetically modifying leukocyte and a method of using these leukocytes.

### SUMMARY OF THE INVENTION

The following embodiments and aspects thereof are described and illustrated in conjunction with compositions and methods which are meant to be exemplary and illustrative, not limiting in scope.

Genetically modified leukocytes, and a composition including genetically modified leukocytes, are provided which contain or express a gene that confers reversible resistance to a glucocorticoid. In various embodiments, the gene that confers reversible resistance to glucocorticoid encodes a 11-beta-hydroxysteroid dehydrogenase, which is a glucocorticoid-degrading enzyme, e.g., corticosteroid 11-beta-dehydrogenase isozyme 2, corticosteroid 11-beta-dehydrogenase isozyme 1, and hydroxysteroid 11-beta-dehydrogenase 1-like protein. Various embodiments provide these genetically modified leukocytes degrade a glucocorticoid (e.g., convert cortisol to cortisone) at a rate of at least 20 pg/hour/10⁵ of genetically modified leukocytes, 50 pg/hour/10⁵ of genetically modified leukocytes, 100 pg/hour/10⁵ of genetically modified leukocytes, 200 pg/hour/10⁵ of genetically modified leukocytes, 300 pg/hour/10⁵ of genetically modified leukocytes, 400 pg/hour/10⁵ of genetically modified leukocytes, 500 pg/hour/10⁵ of genetically modified leukocytes, 600 pg/hour/10⁵ of genetically modified leukocytes, 700 pg/hour/10⁵ of genetically modified leukocytes, 800 pg/hour/10⁵ of genetically modified leukocytes, 900 pg/hour/10⁵ of genetically modified leukocytes, 1000 pg/hour/10⁵ of genetically modified leukocytes, 1200 pg/hour/10⁵ of genetically modified leukocytes, 1500 pg/hour/10⁵ of genetically modified leukocytes, 2000 pg/hour/10⁵ of genetically modified leukocytes, or more; whereas leukocytes without the genetic modification with the mentioned transgene(s) have little (e.g., <20 pg/hour/10⁵ of leukocytes) or undetectable degradation of the glucocorticoid, and whereas an inhibitor of 11-beta-hydroxysteroid dehydrogenase can reduce the degradation rate of the glucocorticoid by respective genetically modified leukocytes at about 10%, 20%, 30%, 40%, 50%, 60%, 70%, 80%, 90% or 100%.

Examples of genes to modify leukocytes include 11-beta-hydroxysteroid dehydrogenase type II (HSD11B2), 11-beta-hydroxysteroid dehydrogenase type III gene variant (HSD11B1L), 11-beta-hydroxysteroid dehydrogenase type I (HSD1B1), and a modified SERPINA6 gene. In some embodiments, the gene to confer reversible resistance to glucocorticoid in leukocytes includes the polynucleotide of HSD11B2, and does not include HSD11B1, HSD11B1L or a modified or non-modified SERPINA6 gene. In other embodiments, the gene to confer reversible resistance to glucocorticoid in leukocytes includes the polynucleotide of HSD11B1, and does not include HSD11B2, HSD11B1L or a modified or non-modified SERPINA6 gene. Preferably, the resistance to glucocorticoid by the leukocyte is reversible by means such as adding an inhibitor of 11-beta-hydroxysteroid dehydrogenase; or the resistance can be overcome by increasing the concentration of glucocorticoid to the leukocytes.

Further aspects provide that the polynucleotide of HSD11B2 to confer reversible resistance to glucocorticoid in leukocytes have different forms or modifications.

One exemplary modification includes codon optimization of the exon 1 of HSD11B2, e.g., as set forth in SEQ ID No.: 18, denoted as “B2” in FIGS. 8A-8C and 9. In one aspect, leukocytes transduced with a vector comprising HSD11B2 with codon-optimized exon 1 (e.g., as set forth in SEQ ID NO.: 18) convert cortisol to cortisone at a rate of about 600-800 pg/hour/10⁵ of genetically modified leukocytes, whereas control leukocytes without transgene or with a transgene that encode product with no interaction with glucocorticoid have little to undetectable conversion of cortisol to cortisone.

Another exemplary embodiment is without codon modification at approximately exon 1 of HSD11B2, which remains “wild type,” e.g., as set forth in SEQ ID No.: 31, denoted as “wtExon1-none” in FIGS. 8A-8C and 9. In one aspect, leukocytes transduced with a vector comprising HSD11B2 without codon modification at exon 1 (e.g., as set forth in SEQ ID No.: 31) convert cortisol to cortisone at a rate of about 800-1200 pg/hour/10⁵ of genetically modified leukocytes, and/or convert prednisolone or dexamethasone at a rate of at least 2000 pg/hour/10⁵ of genetically modified leukocytes, whereas control leukocytes without transgene or with a transgene that encode product with no interaction with glucocorticoid have little to undetectable conversion of cortisol to cortisone.

Yet another exemplary modification is using a bicistronic sequence encoding HSD11B2 and a sequence encoding a cell surface protein including a marker (e.g., “tag”), the two of which are linked with a “2A” sequence that can encode a self-cleaving peptide, denoted as “B2-Tag” (“tag” is downstream of HSD11B2) as shown in FIGS. 8A-8C and 9, e.g., whose polynucleotide sequence is set forth in SEQ ID No.: 32, and the polypeptide sequence is set forth in SEQ ID No.: 33. In one aspect, leukocytes transduced with a vector comprising HSD11B2 with a 2A sequence and a tag downstream (e.g., as set forth in SEQ ID No.: 32) convert cortisol to cortisone at a rate of about 200-600 pg/hour/10⁵ of genetically modified leukocytes, whereas control leukocytes without transgene or with a transgene that encode product with no interaction with glucocorticoid have little to undetectable conversion of cortisol to cortisone.

Another exemplary modification is using a bicistronic sequence encoding HSD11B2 following a sequence encoding a cell surface protein including a tag, the two of which are linked with a “2A” sequence that can encode a self-cleaving peptide, denoted as “Tag-B2” (where the “tag” is upstream of HSD11B2), e.g., an exemplary polynucleotide as set forth in SEQ ID No.: 37, which translates to a polypeptide set forth in SEQ ID No.: 38.

Another embodiment includes a bicistronic sequence encoding (i) HSD11B1 or HSD11B2 and (ii) a cell surface protein that acts to direct the cell to response to the presence of an antigen on another cell.

In one aspect, leukocytes transduced with a vector comprising HSD11B1 (e.g., as set forth in any of SEQ ID Nos.: 17, 19-28) convert cortisol to cortisone at a rate of about 20-200 pg/hour/10⁵ of genetically modified leukocytes, whereas control leukocytes without transgene or with a transgene that encode product with no interaction with glucocorticoid have little to undetectable conversion of cortisol to cortisone.

Various embodiments provide a composition including these genetically modified leukocytes. The composition may be a population of leukocytes wherein at least five, six, seven, eight, nine, or ten percent contain or express a gene that confers reversible resistance to a glucocorticoid. An exemplary embodiment provides lymphocytes that are genetically modified (e.g., transduced) with HSD11B2-containing vector (e.g., HSD11B2 transgene) deplete cortisol, prednisolone or dexamethasone, e.g., reduction in the levels of cortisol, prednisolone or dexamethasone present in the culture media of these genetically modified lymphocytes. A further aspect of this embodiment provides these HSD11B2-transduced lymphocytes act on cortisol to increase cortisone, the inactive metabolite of cortisol, e.g., in cell culture media. A further aspect of this embodiment provides these HSD11B2-transduced lymphocytes act on other steroids to increase the concentrations of 11-keto forms of the steroid. Another aspect of this embodiment provides the conversion from cortisol to cortisone, as well as the depletion of prednisolone and dexamethasone, conferred by HSD11B2 transgene to the genetically modified lymphocytes, is inhibited by an inhibitor of HSD11B2 such as posaconazole. Another exemplary embodiment provides lymphocytes that are genetically modified (e.g., transduced) with HSD11B1-containing vector (e.g., HSD11B1 transgene) deplete cortisol and convert cortisol to cortisone, and deplete prednisolone and/or dexamethasone. Another aspect of this embodiment provides the conversion from cortisol to cortisone, as well as the depletion of prednisolone and dexamethasone, conferred by HSD11B1 transgene to the genetically modified lymphocytes, is inhibited by an inhibitor of HSD11B1 such as posaconazole. The composition may also be a pharmaceutical composition, including a population of the genetically modified leukocytes and a pharmaceutically acceptable diluent or excipient. Various embodiments provide the genetically modified leukocytes are further modified to express a recombinant T-cell receptor gene or a chimeric T cell antigen receptor.

In various embodiments, the leukocytes with genetic modifications to exhibit reversible resistance to glucocorticoid are selected from the group consisting of alveolar macrophages, antigen presenting cells, B-cells, basophils, cytotoxic T-cells, dendritic cells, epithelioid cells, eosinophils, giant cells, granulocytes, helper T-cells, histiocytes, Kupffer cells, Langerhans cells, large granular lymphocytes, leukocyte precursors, lymphocytes, mast cells, memory cells, microglia, monocytes, monoosteophils, myeloid dendritic cells, natural killer cells, natural killer T cells, neutrophils, osteoclasts, phagocytes, plasma cells, plasmacytoid dendritic cells, regulatory T-cells (Tregs), suppressor T-cells, T-cells and tumor infiltrating basophils.

A process of reducing steroid resistance in genetically modified leukocytes is also provided, which includes administering a pharmaceutically effective amount of an 11-beta-hydroxysteroid dehydrogenase inhibitor to the subject having received genetically modified leukocytes that are reversibly resistant to a glucocorticoid. Exemplary inhibitors of 11-beta-hydroxysteroid dehydrogenase (HSD) include carbenoxolone, itraconazole, hydroxyitraconazole (OHI), ketaconazole and posaconazole. In various embodiments, steroid-resistant cells disclosed herein are rendered responsive (subject to) the immunosuppressive effective of steroid in the presence or after treatment with inhibitors of HSD.

A process of providing steroid resistance in a subject in need thereof is also provided, which includes administering a therapeutically effective amount of a population of genetically modified leukocytes that are reversibly resistant to a glucocorticoid. In various aspects, these leukocytes contain or express a 11-beta-hydroxysteroid dehydrogenase.

In some embodiments, a process of treating an inflammatory, auto-immune, infectious, or cancerous disease or disorder includes a combination of providing steroid resistance and administering existing therapeutics. A composition containing the genetically modified leukocytes exhibiting reversible resistance to glucocorticoid may be administered concurrently or sequentially with one or more of glucocorticoid, nonsteroidal anti-inflammatory drugs, anti-infectives, and chemotherapeutics.

A process of improving the in vitro growth of leukocytes expressing a 11-beta-hydroxysteroid dehydrogenase gene is also provided, which includes incubating the cells with an effective amount of an inhibitor of 11-beta-hydroxysteroid dehydrogenase. Preferably, the 11-beta-hydroxysteroid dehydrogenase gene is HSD11B2.

A process of screening an inhibitor capable of reversing glucocorticoid resistance is also provided, which includes contacting a candidate agent with a population of cells including at least five percent genetically modified leukocytes that express a gene that confers resistance to 11-beta-hydroxysteroids; followed by measuring resistance to steroids of the population of cells. Typically, a loss or reduction of resistance to steroids of the population of cells indicates the candidate agent is an inhibitor capable of reversing glucocorticoid resistance; and an absence of the loss or reduction of resistance indicates the candidate agent is not an inhibitor capable of reversing glucocorticoid resistance.

An expression vector containing a gene that confers reversible resistance to a glucocorticoid is provided. In various embodiments, the vector has a backbone of pCCL-c-MNDU3c-X. In various embodiments, the genetically modified leukocytes of the present invention contains an expression vector with a backbone of pCCL-c-MNDU3c-X and an insertion of a nucleic acid sequence that encodes one or more 11-beta-hydroxysteroid dehydrogenase, e.g., preferably HSD11B2. Such an expression vector may be introduced to modify leukocytes by various transfection methods such as electroporation.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts reactions catalyzed by reductase or dehydrogenase activities of HSDs (e.g., HSD11B1 and HSD11B2) on glucocorticoids as exemplified with cortisol and cortisone. 11-beta-hydroxysteroid dehydrogenases act on the side group of the carbon at position 11 of glucocorticoids (circled). This example shows cortisone (left-most structure, keto group at position 11) and cortisol (right-most structure, hydroxyl at position 11). In certain organs and tissues, HSD11B1 can also perform the “reverse” reaction that synthesizes active steroid (e.g., cortisol) from an inactive steroid (e.g., cortisone). (Prior Art.)

FIGS. 2A and 2B depict transfection efficiency. Flow cytometry of lymphoid cells electroporated with vector containing green fluorescent protein (GFP) performed in parallel to electroporation with vectors containing cloned HSD genes. Green fluorescent protein detection in cells electroporated with a GFP containing vector (FIG. 2B) compared to electroporation with non-GFP vector (FIG. 2A) demonstrates the electroporation conditions supported efficient gene transfer.

FIGS. 3A and 3B depict the percentages of survival of RS4;11 cells transduced with HSD11B1 lentivector, in the presence of various concentrations (nM) of dexamethasone (DEX; FIG. 3A) and prednisolone (PRED; FIG. 3B). Wild-type, untransduced RS4;11 cells (given vehicle alone) were used as control.

FIGS. 4A and 4B depict the percentages of survival of RS4;11 cells transduced with HSD11B2 lentivector, in the presence of various concentrations (nM) of dexamethasone (DEX), occasionally with an HSD inhibitor, carbenoxolone at 10 μM or 1 μM (denoted as CBX-10 or CBX-1, respectively; FIG. 4A), or posaconazole at 1 μM (PZ; FIG. 4B). Values expressed as percentage of each cell type's (HSD2 transduced or wild type, un-transduced) control treated with vehicle alone. Data for DEX alone treatments of HSD11B2 and wild type cells are duplicated in FIGS. 4A and 4B for reference. Protection was reversed using 1 μM of posaconazole (PZ) or 10 μM or 1 μM of carbenoxolone (CBX-10, CBX-1).

FIGS. 5A and 5B depict the percentages of survival of RS4;11 cells transduced with HSD11B2 lentivector, in the presence of various concentrations (nM) of prednisolone (PRED), occasionally with an HSD inhibitor, carbenoxolone at 10 μM or 1 μM (denoted as CBX-10 or CBX-1, respectively; FIG. 5A), or posaconazole at 1 μM (PZ; FIG. 5B). Values expressed as percentage of each cell type's (HSD11B2 transduced or wild type, un-transduced) control treated with vehicle alone. Protection was reversed using 1 μM of posaconazole (PZ) or 10 μM or 1 μM of carbenoxolone (CBX-10, CBX-1). Data for PRED alone treatments of HSD11B2 and wild type cells are duplicated in FIGS. 5A and 5B for reference.

FIGS. 6A and 6B depict stimulated leukocytes are predominantly CD3 positive lymphocytes, as determined by flow cytometry. 93.6% of live stimulated leukocytes are CD3 positive.

FIGS. 7A and 7B depicts flow cytometry showing 44.1% of stimulated leukocytes express surface marker antigen detected by Chessie 13-39.1 (“tag”) following two (right panel) rounds of transduction with lentivector containing a bicistonic B2-tag construct compared to control antibody staining (left panel).

FIGS. 8A, 8B and 8C depict the degradation of cortisol, prednisolone (Pred) and dexamethasone (Dex), respectively, by stimulated leukocytes transduced with lentiviral vectors with gene constructs (bar labels): HSD11B1 (“HSD1”), HSD11B2 (“B2”), wtExon1-none (“wt1-B2”), B2-Tag (“B2-Tag”) and THREE-MIX-ALPHA (as control; “C”). Steroid detected by ELISA. Effect of HSD inhibitor posaconazole (Posac; 7 μM) on the degradation. Values are adjusted for number of transgene bearing cells present at the end incubation. Asterisk indicates value is below the limit of quantitation for the assay. Gene constructs HSD11B1, HSD11B2, wtExon1-none (“wt1-B2”) and B2-Tag all degraded the steroids. Control cells had no significant steroid degradation activity. In FIG. 8A, with “wt1-B2,” the cortisol levels at end of assay were below the limit of quantitation by the ELISA, indicating most cortisol was depleted and the depletion rate incalculable from this data, hence the broken bar thereof.

FIG. 9 depicts the conversion of cortisol to cortisone by stimulated leukocytes transduced with lentiviral vectors of gene constructs (bar labels): HSD11B1 (“HSD1”), HSD11B2 (“B2”), wtExon1-none (“wt1-B2”), B2-Tag (“B2-Tag”) and THREE-MIX-ALPHA (“C”; control) measured by UHPLC-Mass Spectrometry. Effect of HSD inhibitor posaconazole (“Posac”) on the conversion. Culture media without cells (“M”) and without added cortisol was also analyzed. Y-axis values are given in pg per hour per 10e5 transgene copies. Asterisk indicates value is below the limit of quantitation for the assay. Gene constructs HSD11B1, HSD11B2, wtExon1-none and B2-Tag all generated cortisone. Production of cortisone by control transduced stimulated lymphocytes (“C”) was below the quantitative limit of the assay.

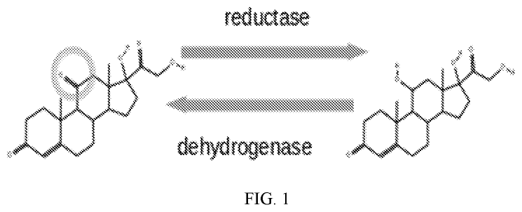

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning. A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

2A self-cleaving peptides, or 2A peptides, is a class of 18-22 aa-long peptides, which can induce the cleaving of the recombinant protein in cell. 2A peptides are derived from the 2A region in the genome of virus. The members of 2A peptides are named after the virus in which they have been first described. For example, F2A, the first described 2A peptide, is derived from foot-and-mouth disease virus. Exemplary 2A peptides include, but are not limited to, P2A, E2A, F2A and T2A, with the following sequences (adding the sequence “GSG” (Gly-Ser-Gly) on the N-terminal of a 2A peptide is optional):

P2A has a sequence of
(SEQ ID No.: 39)
ATNFSLLKQAGDVEENPGP
or
(SEQ ID No.: 40)
GSGATNFSLLKQAGDVEENPGP;
E2A has a sequence of
(SEQ ID No.: 41)
QCTNYALLKLAGDVESNPGP
or
(SEQ ID No.: 42)
GSGQCTNYALLKLAGDVESNPGP;
F2A has a sequence of
(SEQ ID No.: 43)
VKQTLNFDLLKLAGDVESNPGP
or
(SEQ ID No.: 44)
GSGVKQTLNFDLLKLAGDVESNPGP;
and
T2A has a sequence of
(SEQ ID No.: 45)
EGRGSLLTCGDVEENPGP
or
(SEQ ID No.: 46)
GSGEGRGSLLTCGDVEENPGP;

“Adoptive cellular therapy,” “adoptive cell therapy,” or “adoptive cell transfer” (ACT) refers to the treatment of a disease by the adoptive transfer of hematologic cells including leukocytes to a patient whereby the hematologic cells modulate a disease and/or its symptoms. Adoptive cellular therapy includes, but is not limited to, the use of: blood or platelet transfusions; donor-derived anti-viral lymphocytes to treat viral infections; tumor infiltrating lymphocytes (TILs) for cancer treatment; chimeric antigen receptor bearing T-cells (CAR-T) for cancer; lymphocytes that have been selected for, or genetically modified to drive, expression of anti-tumor T-cell receptor genes; natural killer cells for cancer treatment; antigen presenting cells such as dendritic cells or macrophages that present microbial, viral or tumor antigens; hematopoietic stem cell transplantation whereby hematopoietic progenitors are delivered, often contained within populations of bone marrow, peripheral blood (with or without mobilization of hematopoietic precursors), umbilical cord blood or enriched precursor cells (e.g., CD34+ cells); hematopoietic cell grafts with and populations of leukocytes for use in graft-versus-leukemia or graft-versus-tumor responses; transfusions of leukocytes or their precursors to treat acquired or congenital leukopenias and immune deficiencies; leukocytes including CD3+ T-cells to promote immune reconstitution following hematopoietic ablation and hematologic stem cell transplantation. Cells used in ACT may be obtained or derived from the recipient of the ACT (i.e., self or autologous cell population), from another individual or individuals (“non-self”), or some mixture of self and non-self. Cells used in ACT may be genetically modified.

“Adoptive cellular immunotherapy” or “adoptive cell immunotherapy” refers to a type of adoptive cellular therapy where an immune cell is delivered into a mammal to effect a beneficial result. Examples of adoptive cellular immunotherapy include, but are not limited to, anti-viral T cells, CAR-T cells, tumor infiltrating lymphocytes (TILs) and natural killer cells.

“Auto-immune disease,” “auto-immune disorders,” or “auto-immunity” refers to or describes a condition in mammals where an immune response interferes with, or causes damage to normal cells, tissues or physiological processes. Examples of auto-immune disorders include but are not limited to alopecia areata, antiphospholipid antibody syndrome (aPL), autoimmune hepatitis, Celiac disease, diabetes type 1, eosinophilic esophagitis, Graves' disease, Guillain-Barre syndrome, Hashimoto's thyroditis, hemolytic anemia, Idiopathic thrombocytopenic purpura (ITP), Inflammatory bowel disease (IBD), ulcerative colitis, inflammatory myopathies, multiple sclerosis, myasthenia gravis, primary biliary cirrhosis, Rheumatoid arthritis (adult and juvenile), scleroderma, Sjögren's syndrome, Systemic lupus erythematosus (SLE), vitiligo.

“b,” “B,” “beta” and “0” when used as prefixes in definitions of molecules are used equivalently and interchangeably.

“Beneficial results” as used herein may include, but are not limited to, lessening or alleviating the severity of the disease condition, preventing the disease condition from worsening, curing the disease condition, preventing the disease condition from developing, lowering the chances of a patient developing the disease condition, prolonging a patient's life or life expectancy and reducing side-effects.

“Cancer” and “cancerous” refer to or describe a condition in mammals that is typically characterized by unregulated cell growth. Examples of cancer include, but are not limited to carcinomas, sarcomas, B-cell lymphomas (Hodgkin's lymphomas and/or non-Hodgkins lymphomas, Burkitts' lymphoma), leukemias, T-cell lymphomas, multiple myelomas, brain tumor, breast cancer, histiocytosis, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, nasopharyngeal cancer, head and neck cancer, brain cancer, and prostate cancer, including but not limited to androgen-dependent prostate cancer and androgen-independent prostate cancer.

“Co-express” refers to simultaneous expression of two or more genes. Genes may be nucleic acids encoding, for example, a single protein or a chimeric protein as a single polypeptide chain. In an embodiment, the first and second polynucleotide chains are linked by a nucleic acid sequence that encodes a linker polypeptide that is capable of being cleaved. In another embodiment, the first and second polynucleotide chains are driven by independent promoters. In another embodiment the polynucleotides may be linked by internal ribosome entry sequence (IRES) or a functionally equivalent sequence. Alternately, the genes are encoded by two different polynucleotides and are instead present on, for example, two different vectors. If the aforementioned sequences are encoded by separate vectors, these vectors may be simultaneously or sequentially transfected or transduced.

“Conditions,” “disease conditions,” “diseases” and “disease state” include physiological states in which a disease or symptom is manifest. Examples of conditions include cancer, infection, auto-immunity, graft failure and delayed hematopoietic reconstitution.

“Corticosteroids,” “steroids,” “glucocorticoids,” “glucocorticosteroids” or “11-beta-hydroxysteroids” refers to a class of steroid hormones that are produced in the adrenal cortex of vertebrates, as well as the synthetic analogues of these hormones. Natural and synthetic corticosteroids may display glucocorticoid activity, mineralocorticoid activity or both. The classification of a steroid as a “glucocorticoid” or “glucocorticosteroid” is intended to emphasize a compound's predominant glucocorticoid activity even if the compound also has mineralocorticoid activity.

“Effector function” refers to the specialized function of a cell. For example, an effector function of a T-cell may be cytolytic activity, helper activity, suppressor activity, regulatory activity and may include the secretion of cytokines when the cell is stimulated. Effector function may act locally to the cell (e.g. cytolytic activity), distally (e.g. secretion of cytokines) or both locally and distally (e.g. secretion of cytokines).

“Electroporation” refers to the administration of an electric current to a cell or population of cells so that nucleic acid present outside the cell is rapidly brought into the cell. Electroporation is a form of transfection.

“Express” or “expression” refers to the production of a protein either directly from a gene under steady-state conditions or production as a result of induction of expression of that gene by a factor from outside the cell.

“Insert,” “payload” or “gene” refers to a polynucleotide that is delivered into a cell to cause genetic modification. These phrases may also define genes, “foreign” genes or sequences and “transgenes.” Gene refers to polynucleotide that is introduced into a cell, or may refer to a polynucleotide or a site of targeting within a cell. When referring to a site of chromosomal genetic modification, a particular genomic location may be referred to terms including but not limited to a gene, a locus, an allele and may be identified by position on chromosomal maps. Often, but not required, a gene may be capable of producing an RNA transcript or being recognized by DNA or RNA processing machinery (for example a payload comprising an exogenous gene promoter that would be inserted in front of the coding region of a given gene in order to drive gene expression in a situation where otherwise the gene's endogenous promoter would not drive expression). Often, but not required, the payload is delivered as part of a vector. Payload may include regulatory or control sequences, such as start, stop, promoter, signal, disruptive sequences (e.g. sites for homologous recombination), anti-sense sequences, RNA stability or RNA regulation sequences, internal ribosome entry sequences, signal for protein secretion or targeting to organelles, or other sequences used by a cell's genetic, transcriptional and translational machinery. The gene or sequence may include nonfunctional sequences or sequences with no known function. A host cell that receives introduced DNA or RNA has been “transformed” and is a “transformant.” The invention also contemplates DNA sequences that encode the same desired protein by alternative codon usage.

Gene name abbreviations used herein (e.g. SERPINA6, HSD11B2, HSD11B1L, HSD11B1, NR3C1), unless otherwise specified, refer to the human gene. The corresponding genes, names and gene name abbreviations for other species are readily obtained by one skilled in the art to which this invention belongs. To the extent the meaning from the context is recognized by one of ordinary skill in the art, no distinction is made in the nomenclature between the human gene and the human gene product (protein) in the application, as we have not adhered to the HUGO Gene Nomenclature Committee of italicized text for human gene symbols and non-italicized for the human protein.

“Genetically modified cells,” “gene modified cells,” “redirected cells,” or “genetically engineered cells” refer to cells or cell types that have had their complement of DNA or RNA altered by external action. Many methods for such modification are known and include, but are not limited to: transduction of cells using a viral or viral-based vector, transfection of an expression vector (often a plasmid), introduction of enzymes or enzyme systems with additional components whereby those systems modify a cell's DNA and or RNA complement. Such systems include Transcription activator-like effector nucleases (TALENS), Zinc finger proteins and clustered regularly interspaced short palindromic repeats (CRISPR)/Cas9. Other systems genetic modifications approaches use transposons in systems such as Sleeping Beauty. For example, one type of “genetically modified leukocyte” is a leukocyte that contains a gene that confers steroid resistance of the invention and which also co-expresses a transgene encoding for a recombinant T-cell receptor gene or a chimeric T cell antigen receptor. Genetically modified cells may be created by the action of man (e.g. delivery of a protein of, or RNA encoding for, a recombinase or integrase enzyme), by nature (e.g. by a viral infection such as Epstein Barr Virus or a wart virus) or a combination of both man and nature. Genetic modifications also include but are not limited to modifications to the cell's structures around DNA and RNA which includes epigenetic modifications of DNA, RNA (e.g. methylation of nucleotide bases) and post-translational modification of proteins involved in the regulation of the function of DNA and e.g. acetylation or methylation of chromosomal histones.

“Immune cell” refers to a leukocyte which has a direct role in an immune response or which has immune cell function.

“Immune cell function” refers to a cell's known or potential function in an “immune response” and may or may not include other activities which may include, but are not limited to, removal of cellular and tissue debris including enucleation of erythrocytes, maturation of erythroid cells, maturation of platelets, production of cytokines and pro-inflammatory factors, promoting apoptosis or anergy in leukocytes, providing survival signals to leukocytes, immune surveillance, migration, antigen presentation, maintaining an immune system, anti-viral cytotoxicity, anti-cancer cytotoxicity, promoting engraftment of transplanted hematopoietic stem and progenitor cells, anti-helminth activity, phagocytosis of microbes. wound repair, bone repair, promoting immune tolerance and antibody-dependent cell mediated cytotoxicity (ADCC). Immune cell function, like aspects of immune response, may promote the health of a mammal or may be deleterious, for example, by causing auto-immunity or graft rejection.

“Immune response” refers to immune activities including, but not limited to: innate immunity, humoral immunity, cellular immunity, immunity, inflammatory response, acquired (adaptive) immunity, autoimmunity and/or overactive immunity, breaking of immune tolerance, graft rejection, response to allo- and xeno-antigens, graft-versus-leukemia activity, graft-versus-tumor activity, graft-versus-host disease, promoting immune tolerance and includes immune responses produced by adoptive cellular therapies.

“Leukocyte” refers to a cell of the blood cell lineage. Leukocytes include, but are not limited to, alveolar macrophages, antigen presenting cells, B-cells, basophils, cytotoxic T-cells, dendritic cells, epithelioid cells, eosinophils, giant cells, granulocytes, helper T-cells, histiocytes, Kupffer cells, Langerhans cells, large granular lymphocytes, leukocyte precursors, lymphocytes, macrophages, mast cells, memory cells, microglia, monocytes, monoosteophils, myeloid dendritic cells, natural killer cells, natural killer T cells, neutrophils, osteoclasts, phagocytes, plasma cells, plasmacytoid dendritic cells, regulatory T-cells (Tregs), suppressor T-cells, T-cells and tumor infiltrating basophils. Leukocytes are distinguishable from two other lineages of the blood cells—erythroid cells (wherein maturing erythrocytes contain substantial levels of hemoglobin protein) and megakaryocytes and platelets. Leukocyte as used herein refers to a non-erythroid, non-megakaryocytic hematologic cell regardless of whether the leukocyte has been derived from a normal physiological hematopoietic process of a mammal, e.g. is a cell of, or is a cell derived from, a sample obtained from a human patient or donor, or whether the leukocyte was generated from an alternate population of cells, such as, and not limited to, a leukocyte generated in vitro from precursors or progenitors derived from other sources of cells including, but not limited to, embryonic stem cells (ESC) or induced pluripotent stem cells (iPSC). The forgoing definitions are not limiting—other cell populations with lesser potency (multi-potent, bi-potent or uni-potent) may be used as sources of leukocyte precursors or progenitors instead of pluripotent or totipotent cells. The forgoing cell populations cells may be transformed. Mature and maturing leukocytes may be found throughout the human body including without limitation, circulating in the peripheral blood (e.g. neutrophils, eosinophils, basophils, T-cells, B-cells, macrophage/monocytes, dendritic cells), in the lymphatics, the spleen, the liver, in the primary and secondary lymphoid organs and in the central nervous system. Leukocytes may be resident in tissues (e.g. microglia in the central nervous system, tissue macrophages or osteoclasts in bone tissue). Some leukocytes migrate and traffic through tissues and organs and adopt new phenotypes depending on their history, function and location, by way of example monoosteophils being produced from the monocytes and macrophage lineage. Macrophages may fuse to give rise to giant cells. Although the anatomical location of a leukocyte may give clues to the type of leukocyte or its function, classification of a leukocyte requires a multi-parametric analysis based on definitions in the field for each leukocyte sub-population that are in use at the time of analysis.

Sub-populations of leukocytes are generally defined by parameters such as cell size, shape, intracellular granularity and degree of surface regularity, cell surface markers, gene expression including specialized genes such as the T-cell receptor, immunoglobulin heavy and light chains, and cellular function. These parameters can be examined by many means known in the field including, but not limited to, electromagnetic radiation including optically by cell analyzers, light microscopy with and without histological stains, the use of antibodies, aptamers and with means of detection (e.g. fluorochromes, quantum dots, enzyme staining) in combination with techniques such as cell imaging, flow cytometry or mass-spectroscopy-cytometry, analysis of intracellular markers by assays including granule types and contents, enzyme function, permeabilization for antibody or aptamer staining of intracellular antigens including cytokines, functional studies (e.g. phagocytosis, motility and chemotaxis, degranulation, capacity to undergo mitosis in response to cytokines or in response to stimuli such as aggregation of cell surface antigens by cross-linking antibodies or by mitogens, ability to stimulate, suppress or attract other leukocytes, ability to kill target cells or kill or ingest pathogens, ability to degrade, remodel or form bone), gene expression analysis, impedance analysis and cell adhesion noise (CAN-Q), adherence to substrates including plastic or antibody coated beads or columns. Leukocyte precursors can be defined using the same parameters as listed herein, plus additional studies that may be performed to evaluate the potency of such precursors—including which cell types can be produced from the precursor cell and the precursor's proliferation potential—using in vitro or in vivo studies. T-cells and B-cells are examples of leukocytes where sub-populations of these cells continue to be identified. Leukocytes that have undergone ex-vivo manipulation may display different phenotypes when compared to the original cells or other leukocytes. This phenotypic difference may be particularly evident when leukocytes are maintained in culture for hours, days or weeks, including under culture conditions that drive mitosis.

In another aspect, leukocyte as used herein also refers to a cell obtained from a leukocytic leukemia, lymphoma, histiocytosis or dysplasia. In yet another aspect, leukocyte as used herein also refers to a cell of a cell line, regardless of whether that cell line is stable or unstable, transformed or untransformed, where that cell line is derived from leukocytes, leukocyte precursors or leukocyte progenitors. Examples of leukocyte cell lines used for human clinical studies include “GRm13Z40-2”, a cytotoxic T-lymphocyte cell line genetically modified by the targeted disruption of GR alleles and Neukoplast (NK-92), a natural killer cell line.

In various aspects, the definition of leukocyte and classification of leukocyte type also anticipates that some mammalian leukocytes and the precursors of some hematologic lineages may display plasticity, i.e. an ability to develop and differentiate, or de-differentiate, between two or more lineages that were otherwise believed to be distinct paths of development and maturation, e.g. see “Transdifferentiation of Malignant B-Cells into Macrophages in a Murine Model of Burkitt's Lymphoma”, Bruns et al. (2014) Blood, vol. 124 no. 21 5406 and references therein.

“Mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Patient” as used herein refers to a mammal.

“Polynucleotide” includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), anti-mirs (also known as antagomirs), small activating RNAs (saRNAs), genomic DNA, synthetic DNA, synthetic RNA, and/or tRNA and hybrids between a strand of DNA and a strand RNA form.

“Resistance” refers to reducing the effectiveness of a drug, compound or other condition acting on a cell, whereby a cell which has resistance (“resistant cell”) shows a lower responsiveness to the drug, compound or condition when compared to a non-resistant cell. Resistance may be overcome or reversed by reducing, eliminating or defeating the mechanisms which underpin resistance.

“Responsiveness” refers to the measurable response of a cell. Techniques used to measure responsiveness include but are not limited to: performing laboratory analyses and assays including but not limited to measuring the number of cells, the persistence of cells, the growth of cells, the survival of cells, evaluating the cell's ability to migrate; evaluating the cell's ability to interact with target cells and effects on growth, gene expression, cytokine production, phenotype, report gene activity and cell function. Responsiveness can also be evaluated in vivo by, for example, clinical laboratory measurements and observing clinical outcomes.

“Reversible” with respective to glucocorticoid resistance generally refers to the ability to modulate the resistance to glucocorticoid that is conferred by leukocytes that are genetically modified with a transgene of 11-beta hydroxysteroid dehydrogenase. For example, adding an inhibitor of 11-beta-hydroxysteroid dehydrogenase to the genetically modified leukocytes, or subjects administered with the genetically modified leukocytes, can reduce the steroid resistance conferred on the genetically modified leukocytes. As another example, the resistance can be overcome by increasing the concentration of glucocorticoid to the leukocytes, or to the subjects administered with the leukocytes. As another example, the resistance can be overcome by administering a glucocorticoid to the leukocytes, or to the subjects administered with the leukocytes, where such glucocorticoid is a relatively poor substrate for the chosen 11-beta hydroxysteroid dehydrogenase, so that the glucocorticoid not inactivated by the HSD and can reach concentrations within the target cell which produce a desired response.

“Target cell” refers to a cell that is the target of a treatment, immune response or immune cell function. Without limiting the forgoing, by way of some examples, a target cell may be a cancer cell (the target of a cytolytic T cell), a T cell (the target of a suppressor T cell), a T cell (the target of an antigen presenting cell), a B cell (the target of a T helper cell), a transformed epithelial cell i.e. a wart cell (the target of ointment containing anti-wart compound).

The terms “T-cell” and “T-lymphocyte” are interchangeable and used synonymously herein. Without limiting the forgoing, by way of some examples include but are not limited to naive T cells, central memory T cells, effector memory T cells, memory T cells, regulatory T cells, suppressor T cells or combinations thereof.

The terms “transduction” and “transfection” are defined separately herein but share a common basis in delivering a polynucleotide into a cell and are often used synonymously herein. We refer to transfection as a form of polynucleotide delivery that utilizes viruses, viral vectors or components of viruses. “Transduction” refers to the introduction of an exogenous polynucleotide into a cell using a viral vector or components of viruses. “Transfection” refers to the introduction of a exogenous polynucleotide into a cell using a non-viral means. The term “transformation” means the introduction of a polynucleotide comprising a DNA or RNA sequence to a host cell. Transformation may result in the host cell replicating the DNA or RNA sequence, or may result in expression of the introduced DNA or RNA sequence to produce a desired substance, such as a protein or enzyme coded by the introduced DNA or RNA sequence or may simply result in the action of DNA or RNA—without replication or expression—on the DNA or RNA complement of the cell. An example of the latter is the delivery of anti-sense and RNA interference oligonucleotides. The term “transformant” means the cell which has been transformed. A transformant may be a microbe or animal cell. The polynucleotide e.g., DNA or RNA introduced to a host cell can come from any source, including cells of the same genus or species as the host cell, or cells of a different genus or species. A physical process or chemical agent may be applied to assist with the delivery of a polynucleotide. Such physical processes include electroporation and such chemical agents include liposomes and chemical agents that associate with polynucleotides to promote for uptake into cells by endocytosis (including receptor mediated endocytosis), phagocytosis, pinocytosis, emperipolesis and vesicle fusion.

In various aspects, “transformed” has two meanings. One related to transfection, above, the second meaning defined below. In combination, this can lead to sentences wherein both definitions are in use, by way of example and with explanations in square brackets: “we can transfect a cell with a vector and transfection agent wherein that vector contains a transforming oncogene [useful to generate a transformed cell], leading to a transformant [cell post-gene delivery] and resultant transformation [process] of the target cell [into a transformed cell]”. “Transformed” as used herein has a second meaning with respect to cells whereby a transformed cell is a cell that has undergone a genetic or phenotypic change to permit sustained growth in tissue culture or a system of animal passage, where such a transformed cell may display one of more properties of: tumor formation with or without spread, growth factor independence, colony formation, ability to undergo serial passaging in culture and loss of contact inhibition. The process by which a cell is transformed here is “transformation”. One form of transformation is malignant transformation. A transformed cell is often associated with genetic changes, and such changes may be induced by an external action (e.g. by a chemical, physical (e.g., alpha particle) or energetic (e.g. X-ray, gamma ray) mutagen, by a virus or vector containing one or more genes capable of promoting transformation, by fusion with an already transformed cell), by spontaneous genetic re-arrangements or mutations within a cell to result in a transformed phenotype, or by a combination of external action and spontaneous genetic re-arrangements or mutations within a cell.

“Treatment” and “treating” refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented. Treatment also includes medical intervention to reduce side effects of a previous or concurrent treatment, or the selection of a treatment regimen that is expected to minimize side effects.

“Tumor” refers to all neoplastic cell growth and proliferation, whether malignant or benign, and all pre-cancerous and cancerous cells and tissues.

“Vector,” “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc. Viral vectors which may be used include but are not limited to lentiviral vectors, retroviral vectors, foamy virus vectors, adeno-associated virus (AAV) vectors, hybrid vectors and/or plasmid transposons (for example sleeping beauty transposon system) or integrase based vector systems. Other vectors that may be used in connection with alternate embodiments of the invention will be apparent to those of skill in the art.

Genes that Confers Resistance to Glucocorticoids

In various embodiments, genes or alleles that confer resistance to glucocorticoids include HSD11B2 (11-beta-hydroxysteroid dehydrogenase 2), genes encoded by the HSD11B1L family (11-beta-hydroxysteroid dehydrogenase), HSD11B1 (11-beta-hydroxysteroid dehydrogenase 1), SERPINA6 (encoding protein transcortin of SEQ ID NO: 2), and a modified SERPINA6 gene called SER6mod (which encodes protein of SEQ ID NO: 1).

Table 1 summarizes three enzymes with glucocorticoid-degrading activity.

TABLE 1
Brief summary and comparison of three enzymes with glucocorticoid-degrading
activity.

| Gene name | | | |
|---|---|---|---|
| (abbreviation) | HSD11B2 | HSD11B1L | HSD11B1 |
| Protein name | Corticosteroid 11- | Hydroxysteroid 11-beta- | Corticosteroid 11-beta- |
| (UNIPROT | beta-dehydrogenase | dehydrogenase 1-like | dehydrogenase isozyme 1 |
| database) | isozyme 2 (SEQ ID | protein (SEQ ID NO: 5- | (SEQ ID NO: 4) |
| | NO: 3) | 14 for various isoforms | |
| | | or of chimpanzee origin) | |
| Co-factor | nicotinamide adenine | NAD | nicotinamide adenine |
| | dinucleotide | | dinucleotide phosphate |
| | (NAD+) | | hydrogen (NADPH) (to |
| | | | synthesize steroids) |
| | | | nicotinamide adenine |
| | | | dinucleotide phosphate |
| | | | NADP+ (to degrade |
| | | | steroids). |
| Enzyme function | Almost exclusively | Isoform b reported to | Typically reduces steroids |
| | acts as a | display weak | but can perform the reverse |
| | dehydrogenase. | dehydrogenase activity | reaction i.e. |
| | | in vitro at physiological | dehydrogenation under |
| | | pH. | some conditions. |

These three enzymes are further described in Yang et al, Placenta 46 (2016) 63-71; in Chapman et al, Physiol Rev 93: 1139-1206, 2013; and in Gomez-Sanchez and Gomez-Sanchez, Compr Physiol. 2014 July; 4(3): 965-994.

The discovery and analysis of HSD11B1L (also known as SCDR10B) was described in Huang et al., Acta Biochemica Polonia, Vol. 56 No. 2 (2009), 279-289. Specifically, HSD11B2, HSD11B1L and HSD11B1 act on the carbon-I position of a glucocorticoid molecule and either dehydrogenate (convert a hydroxyl group to a keto group) or reduce (convert a keto group to a hydroxyl group). For glucocorticoids, compounds with a hydroxyl group at position 11 (11-hydroxy) demonstrate greater action via the glucocorticoid receptor than compounds with an 11-keto group when tested in vitro. In this manner, cortisol (hydroxyl at carbon 11) has greater activity than cortisone (keto at position 11). By extension, dehydrogenation of a glucocorticoid by an enzyme with 11-beta-hydroxysteroid dehydrogenase activity will generate a less active glucocorticoid, and thus render a cell which expresses the enzyme with steroid 11-beta-hydroxysteroid dehydrogenase activity less responsive or resistant to glucocorticoids.

Another gene that binds to glucocorticoids and which can reduce the concentration of free glucocorticoids within a cell—transcortin—is encoded by the gene SERPINA6, and transcortin protein is expressed and secreted from cells.

In various embodiments, an engineered form of transcortin is provided which, unlike native transcortin protein that is secreted from cells, remains intra-cellular and sequesters glucocorticoids within the cell so as to prevent their binding to cytoplasmic GR. In some aspects, the region encoding the signal peptide that promotes extra-cellular secretion is deleted from the SERPINA 6 gene. This modified SERPINA6 gene is herein termed SER6mod. SER6mod encodes a protein (SEQ ID NO:1) which comprises amino acids 23 through 405 of transcortin (encoded by gene SERPINA6) wherein the numbering refers to the native, full-length protein sequence (SEQ ID NO:2) encoded by SERPINA6 (Uniprot reference for SERPINA6 P08185). The protein produced by SER6mod is not expected to be antigenic because the normal processing of SERPINA6 results in cleavage of the signal sequence. Thus, circulating plasma transcortin is identical to SER6mod protein, except that the SER6mod protein will, in this invention, not be secreted and remain intracellular.

Unlike 11-beta-hydroxysteroid dehydrogenases which act enzymatically to produce reduced responsiveness to glucocorticoids over a wide range of concentrations, glucocorticoid-binding protein encoded by SER6mod, (SEQ ID NO:1), has a level at which its steroid binding capacity becomes saturated, or where such binding to transcortin/SER6mod is essentially avoided by, for example, administering dexamethasone which displays a lower affinity for transcortin compared to other glucocorticoids such as cortisol, prednisone and prednisolone. As such, the resistance conferred by SER6mod in a cell may be overcome by increasing the dose of glucocorticoids, particularly dexamethasone, which have a lower affinity for transcortin and for protein encoded by SER6mod.

Further aspects provide that the polynucleotide of HSD11B2 to confer reversible resistance to glucocorticoid in leukocytes have different forms or modifications. One exemplary modification includes codon optimization of the exon 1 of HSD11B2, e.g., as set forth in SEQ ID No.: 18. Another exemplary embodiment is without codon modification at approximately exon 1 of HSD11B2, which remains “wild type,” e.g., as set forth in SEQ ID No.: 31. Yet another exemplary modification is using a bicistronic sequence encoding HSD11B2 and a sequence encoding a cell surface marker (e.g., “tag”), the two of which are linked with a “2A” sequence that can encode a self-cleaving peptide, denoted as “B2-Tag”, e.g., polynucleotide sequence set forth in SEQ ID No.: 32 and polypeptide sequence set forth in SEQ ID No.: 33.

### Vector/Transfection

The genes may be incorporated into vectors, along with control or other sequences, and used to transfect or transduce cells. The choice of vector and expression control sequences to which HSD11B2, HSD1B1L, HSD11B1 or SERPINA6 is operably linked depends on the functional properties desired, e.g., protein expression, and the host cell to be transformed.

In various embodiments, a gene conferring resistance to glucocorticoid is inserted in a backbone vector pCCL-c-MNDU3c-X of SEQ ID NO: 30. In some aspects, HSD11B2, HSD11B1L or HSD11B1 are codon optimized with additional 5′ and 3′ sequences which correspond to the vector sequence(s) adjacent to restriction enzyme cleavage site(s). In some aspects, the gene for HSD11B2, HSD11B1L or HSD11B1 is edited to have a common particle Kozak consensus sequence. In various aspects, insert genes are synthesized as GBLOCKS® gene fragment.

Appropriate transcriptional/translational control signals and protein coding sequences are described in, for example, Sambrook et al., Molecular Cloning: A Laboratory Manual, 3d Ed. (Cold Spring Harbor Laboratory 2001). These techniques may include in vitro recombinant DNA and synthetic techniques and in vivo recombination, e.g., in vivo homologous recombination. Expression of a nucleic acid sequence may be regulated by a second nucleic acid sequence that is operably linked to the polypeptide-encoding sequence.

Exemplary expression control elements useful for regulating the expression of an operably linked coding sequence include, but are not limited to, inducible promoters, constitutive promoters, secretion signals, and other regulatory elements. When an inducible promoter is used, it can be controlled, e.g., by a change in nutrient status, or a change in temperature, in the host cell medium.

Expression vectors capable of being replicated in a bacterial or eukaryotic host comprising a nucleic acid encoding a polypeptide (or protein) are used to transfect a host and thereby direct expression of such nucleic acid (e.g., genes that confer resistance to glucocorticoid) to produce the polypeptide (or protein). Exemplary mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Transfection methods may include chemical means, e.g.; calcium phosphate, DEAE-dextran, or liposome; or physical means, e.g., microinjection or electroporation. In some embodiments, electroporation is used for transfecting leukocytes with an expression vector containing the insert of HSD11B2, HSD11B1L or HSD11B1.

The transfected cells are grown up by techniques such as those described in Kuchler et al. (1977) Biochemical Methods in Cell Culture and Virology. In various embodiments, the host cell line is mammalian origin, and particularly, human origin.

Numerous expression vector systems may be employed. For example, one class of vector utilizes DNA elements which are derived from animal viruses such as bovine papilloma virus, polyoma virus, adenovirus, adeno-associated virus, herpes simplex virus-1, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. The neomycin phosphotransferase (neo) gene is an example of a selectable marker gene. Additional elements may also be needed for optimal synthesis of mRNA. These elements may include signal sequences, splice signals, as well as transcriptional promoters, enhancers, and termination signals. Examples of expression vectors compatible with eukaryotic cells include pSVL and pKSV-10 (Pharmacia), pBPV-1, pML2d (International Biotechnologies), pTDT1 (ATCC® 31255) and other eukaryotic expression vectors.

The recombinant expression vectors may carry sequences that regulate replication of the vector in host cells (e.g., origins of replication) and selectable marker genes. It will be appreciated by those skilled in the art that the design of the expression vector, including the selection of regulatory sequences may depend on such factors as the choice of the host cell to be transformed, the level of expression of protein desired, etc. Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdmP)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615, which are incorporated herein in their entireties.

### Genetically Modified Leukocytes

Various embodiments provide genetically modified leukocytes that have a resistance to glucocorticosteroid, where the resistance is reversible or can be overcome in a subject receiving the modified leukocytes. The genetically modified leukocytes contain an expression vector of a gene that confers resistance to glucocorticoid. In various aspects, the genetically modified leukocytes contain an expression vector for one or more genes of HSD11B2, HSD11B1L, HSD11B1 and SERPINA6. In some aspects, the genetically modified leukocytes contain an expression vector for HSD11B2, but do not contain an expression vector for HSD11B1L, HSD11B1 or SERPINA6.

Other aspects of the invention provide genetically modified leukocytes that express one or more of corticosteroid 11-beta-hydroxysteroid dehydrogenase isozyme 2, hydroxysteroid 11-beta-hydroxysteroid dehydrogenase 1-like protein, corticosteroid 11-beta-hydroxysteroid dehydrogenase isozyme 1, and a truncated transcortin (SER6mod). In some aspects, genetically modified leukocytes express 11-beta-hydroxysteroid dehydrogenase isozyme 2, but do not express 11-beta-hydroxysteroid dehydrogenase 1-like protein, 11-beta-hydroxysteroid dehydrogenase isozyme 1, or transcortin.

The genetically modified leukocytes with reversible resistance to glucocorticoids provide beneficial results to patients treated with glucocorticoids. Exemplary beneficial results include, or are characterized by, improved leukocyte survival and/or activity in the presence of glucocorticoid compared to native leukocytes or leukocytes without genetic modification of conferring glucocorticoid resistance.

In various embodiments, at least 5%, 6%, 7%, 8%, 9%, 10%, 15%, 20% or 25% of a population of genetically modified cells are genetically modified leukocytes containing genes conferring resistance to glucocorticoids. In various embodiments, about 5%, 6%, 7%, 8%, 9%, 10%, 15%, 20% or 25% of a population of genetically modified cells are genetically modified leukocytes containing genes conferring resistance to glucocorticoids. In various embodiments, up to 5%, 6%, 7%, 8%, 9%, 10%, 15%, 20% or 25% of a population of genetically modified cells are genetically modified leukocytes containing genes conferring resistance to glucocorticoids.

In another embodiment, at least 10% of a population of genetically modified cells are genetically modified leukocytes containing genes conferring resistance to glucocorticoids. In some embodiments, a vector containing the gene that confers resistance to glucocorticoid is electroporated into leukocytes.

Leukocytes can migrate into and out of the peripheral blood pool by a process called extravasation. Lymphocytes may also be retained in the lung pools. Levels of leukocytes in the peripheral blood are related to, and lag behind, cycles in the levels of serum cortisol. Dosing with pharmaceutical steroids results in a decrease of peripheral blood leukocytes including lymphocytes. Thus, serum glucocorticoids can stimulate the extravasation of leukocytes out of the peripheral blood pool. Steroid resistant leukocytes will have a lower response to such stimuli and will persist in the bloodstream for longer duration.

Lymphoid leukemia and lymphomas often involve lymphoid organs such as the spleen and lymph nodes. Steroid-driven extravasation of lymphocytes into the lymphatics may drive adoptive cell therapies (ACTs) into those lymphoid tissues to an extent that the ‘increased local dose’ may be sufficient to offset any reduction in cytotoxicity caused by steroids. This may contrast with solid tumors where, presumably, therapeutic cells are delivered to the target tumor by the bloodstream. ACT tumor-targeting of solid tumors, including by CAR-Ts, may be reduced by steroid-induced extravasation of the cells into lymphatics. In contrast, steroid-resistant lymphocytes and CAR-Ts will persist longer in the peripheral blood as a result of lack of steroid signaling that would otherwise drive their extravasation into secondary lymphoid organs or other such compartments. As a result, steroid-resistant leukocytes including ACTs and CAR-Ts will remain in the bloodstream longer, have more cumulative “dwell time” at tumor sites and thus have increased anti-tumor effectiveness. The forgoing is not limited to modulating ACT's extravasation into the lymphatics and may include modulation of the entry of leukocytes into other sites of leukocyte accumulation including the lung, intestines, omentum, bone marrow and joints including synovial space. The forgoing is also not limited to anti-cancer and may be useful to treat auto-immune disorders, including those involving accumulations of leukocytes in tissues.

### Coexpression System/Therapy

Genetically modified leukocytes generally contains a gene that confers steroid resistance of the invention, and in various embodiments, also co-expresses a transgene encoding an additional therapeutic effect. In some embodiments, the modified leukocytes of the present invention also co-expresses a transgene encoding for a recombinant T-cell receptor (TCR) gene or a chimeric T cell antigen receptor. In one aspect, (1) the polynucleotide chain conferring steroid resistance and (2) the polynucleotide chain encoding a recombinant TCR or a chimeric T cell antigen receptor, are linked by a nucleic acid sequence that encodes a cleavable linker. In another aspect, (1) the polynucleotide chain conferring steroid resistance and (2) the polynucleotide chain encoding a recombinant TCR or a chimeric T cell antigen receptor, are driven by independent promoters. In another aspect, the polynucleotides (1) and (2) may be linked by internal ribosome entry sequence (IRES). In yet another aspect, the polynucleotides (1) and (2) are present on, for example, two different vectors, which may be simultaneously or sequentially transfected or transduced. In another aspect, a combination gene may be used wherein polynucleotides (1) and (2) flank a polynucleotide sequence that encodes a cleavable polypeptide linker such that expression of the combination gene results in a single long polypeptide that is then cleaved into two polypeptides by cleavage of the polypeptide linker.

### Priming/Stimulation of the Leukocytes

In various embodiments, the genetically modified leukocytes of the present invention are also primed or stimulated with an antigen to confer an additional therapeutic effect. In some embodiments, the leukocytes are transfected and thereafter stimulated/primed with the antigen; in other embodiments, the leukocytes are first stimulated/primed with the antigen and then transfected with the gene that confers steroid resistance; and in yet other embodiments, the leukocytes are concurrently stimulated/primed with the antigen and transfected with the gene that confers steroid resistance.

### Pharmaceutical Composition and Dosage

A pharmaceutical composition is also provided including a population of genetically modified leukocytes and a pharmaceutically acceptable carrier or diluent. To facilitate administration, genetically modified leukocytes according to the invention can be made into a pharmaceutical composition or made into an implant appropriate for administration in vivo, with appropriate carriers or diluents, which further can be pharmaceutically acceptable. The means of making such a composition or an implant have been described, for instance, Remington's Pharmaceutical Sciences, 16th Ed., Mack, ed. (1980).

In some aspects, the genetically modified leukocytes can be formulated into a preparation in semisolid (e.g., encapsulated in hydrogel) or in liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not ineffectuate the cells expressing an HSD. Thus, genetically modified leukocytes, including T cells, can be made into a pharmaceutical composition containing a balanced salt solution, for example, Hanks' balanced salt solution, or normal saline.

Various embodiments provide the genetically modified leukocytes, or a pharmaceutical composition thereof, of the present invention can be provided in unit dosage form wherein each dosage unit, e.g., an injection, contains a predetermined amount of the population of genetically modified leukocytes or the composition, alone or in appropriate combination with other active agents. The term unit dosage form refers to physically discrete units suitable as unitary dosages for human and animal subjects, each unit containing a predetermined quantity of the genetically modified leukocytes of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the population of genetically modified leukocytes, or its pharmaceutical composition, in the particular subject.

For example, a single dosage contains about 1×10⁴, 5×10⁴, 1×10⁵, 5×10⁵, 1×10⁶, 5×10⁶, 1×10⁷, or 5×10⁷ of genetically modified leukocytes that contain a gene conferring resistance to glucocorticoid per kilogram of patient bodyweight. One or more dosage units may be administered to a subject depending on the condition of the subject and the therapeutic effects of the treatment. Multiple dosages may be administered at weekly, monthly or yearly intervals, where appropriate.

### Combination Therapy

In various embodiments, genetically modified leukocytes containing a gene that confers resistance to glucocorticoid are used or administered to a subject, in combination with glucocorticoid, nonsteroidal anti-inflammatory drugs, anti-infectives, or chemotherapeutics. The combination of therapies is used to treat, reduce the severity or likelihood, or slow the progression of auto-immune diseases or disorders, inflammatory disorders, infectious diseases or cancers.

In some aspects, genetically modified leukocytes exhibiting resistance of glucocorticoid, or a population thereof, are administered prior to glucocorticoid, nonsteroidal anti-inflammatory drugs, anti-infectives, or chemotherapeutics. In some aspects, genetically modified leukocytes exhibiting resistance of glucocorticoid, or a population thereof, are administered concurrently with glucocorticoid, nonsteroidal anti-inflammatory drugs, anti-infectives, or chemotherapeutics. In other aspects, modified leukocytes exhibiting resistance of glucocorticoid, or a population thereof, and glucocorticoid, nonsteroidal anti-inflammatory drugs, anti-infectives, or chemotherapeutics, are administered repeatedly as needed.

Exemplary glucocorticoids that may be administered concurrently or sequentially with the genetically modified leukocytes include, but are not limited to, cortisol, cortisone, prednisone, prednisolone, methylprednisolone, dexamethasone, betamethasone, triamcinolone, fludrocortisone acetate, and deoxycorticosterone acetate.

Exemplary nonsteroidal anti-inflammatory drugs (NSAIDs) that may be administered concurrently or sequentially with the genetically modified leukocytes include, but are not limited to, aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

Exemplary anti-infectives that may be administered concurrently or sequentially with the genetically modified leukocytes include, but are not limited to, antibiotics, antifungals, anthelmintics, antimalarials, antiprotozoals, antituberculosis agents, and antivirals.

Exemplary chemotherapeutics that may be administered concurrently or sequentially with the genetically modified leukocytes include, but are not limited to, alkylating agents (e.g., mechlorethamine, cyclophosphamide, melphalan, chlorambucil, ifosfamide and busulfan; N-Nitroso-N-methylurea (MNU), carmustine (BCNU), lomustine (CCNU) and semustine (MeCCNU), fotemustine and streptozotocin; dacarbazine, mitozolomide and temozolomide; thiotepa, mytomycin and diaziquone); antimetabolites (e.g., anti-folates, fluoropyrimidines, deoxynucleoside analogues and thiopurines); anti-microtubule agents (e.g., vinorelbine, vindesine, and vinflunine); topoisomerase inhibitors (e.g., irinotecan, topotecan, camptothecin, etoposide, doxorubicin, mitoxantrone, teniposide, novobiocin, merbarone, and aclarubicin); and cytotoxic antibiotics (e.g., doxorubicin, daunorubicin, leukocyteepirubicin, idarubicin, pirarubicin, aclarubicin, mitoxantrone, actinomycin, bleomycin, and mitomycin).

### Methods of Using the Cells

### Providing Glucocorticoid Resistance

In various embodiments, a method of treating, reducing the severity or likelihood, or slowing the progression of a disease in a mammal includes administering a pharmaceutical composition that contains genetically modified leukocytes that express a gene that confers resistance to glucocorticoid. In some aspects, the method of treating, reducing the severity or likelihood, or slowing the progression of a disease in a mammal includes administering a pharmaceutical composition that contains genetically modified leukocytes that express a gene that confers resistance to 11-beta-hydroxysteroids.

Exemplary diseases or disorder to be treated or reduced severity or likelihood of by administering genetically modified leukocytes include neoplasms (e.g. cancers, leukemias and lymphomas), infections caused by microorganisms or viruses (e.g. human cytomegalovirus (CMV), BK-virus, adenovirus), auto-immune disorders (e.g. type I diabetes, systemic lupus erythromatosis (SLE), Hashimoto's thyroiditis), acquired or congenital immune-deficiencies and hematopoietic stem cell transplantations. Examples of autoimmune diseases that may be treated with the genetically modified leukocytes of the present invention include, but are not limited to, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, peraiciousanemia, rapidly progressive glomerulonephritis, psoriasis eosinophilic esophagitis and fibrosing alveolitis. Exemplary diseases or disorder to be treated or reduced severity or likelihood of by administering genetically modified leukocytes along with treating or pre-treating the patient with glucocorticoids include side-effects of delivering immune cells to patients, cytokine release syndrome, severe inflammatory syndrome, vascular leak, hypotension, pulmonary edema, neurotoxicity, cerebral edema, hemophagocytic lymphohistiocytosis or macrophage activation syndrome and mast cell activation syndrome.

### Patient Population

In some embodiments, a subject suitable for receiving the genetically modified leukocytes of the present invention is a patient receiving transplantation, who typically are prescribed with or are taking glucocorticoids to prevent rejection of the graft by components of the immune system. For example, in allogeneic kidney transplantation, patients may be medicated with immunosuppressive drugs such as cyclosporine, sirolimus, mycophenolate mofetil or tacrolimus in combination with a glucocorticoid to prevent rejection of the graft by components of the immune system. Immunocompromised patients are at risk of developing uncontrolled viral infections such as BK virus (BKV) and human cytomegalovirus (CMV). For example, recipients of bone marrow (BMT) or renal transplants, may experience severe renal and urological complications from BKV. There is currently no effective therapeutic for BKV, and the only treatment available in solid organ transplantation is a reduction of immunosuppressive treatment—including glucocorticoids—with the concomitant risk of graft rejection.

### Reducing Glucocorticoid Resistance (Reverse/Overcome Resistance)

In some embodiments, a method is provided of reducing glucocorticoid resistance in a mammal by administering a therapeutically effective amount of an 11-beta-hydroxysteroid dehydrogenase inhibitor to the mammal. In further aspects, an 11-beta-hydroxysteroid dehydrogenase inhibitor reduces glucocorticoid resistance in leukocytes.

Exemplary 11-beta-hydroxysteroid dehydrogenase inhibitors include but are not limited to albendazole, butoconazole, hydroxyitraconazole, itraconazole, ketaconazole, sertaconazole, terconazole, tioconazole, posaconazole, BVT2733, progesterone, 11-beta hydroxyprogesterone, deoxycorticosterone, abietic acid, Merck-544/T0504, carbenoxolone and glycerrhetinic acid (See Chapman et al, Physiol Rev 93: 1139-1206, 2013; Beck et al., Biochem Pharmacol. 2017 Apr. 15; 130:93-103.)

In other embodiments, a method is provided for treating, reducing the severity, or slowing the progression of a side effect resulting from a prior administration of genetically modified leukocytes that express a gene that confers resistance to 11-beta-hydroxysteroids. The method includes administering a therapeutically effective amount of an 11-beta-hydroxysteroid dehydrogenase inhibitor and a glucocorticoid. Exemplary glucocorticoids include but are not limited to beclomethasone, betamethasone, budesonide, cortisol, cortisone, deoxycorticosterone, dexamethasone, hydrocortisone, fludrocortisone, methylprednisolone, prednisolone, prednisone, and triamcinolone.

In various other embodiments, the resistance to glucocorticoids of genetically modified leukocytes expressing, for example SER6mod, is overcome by administration to the patient of an increased level of glucocorticoids, including dexamethasone.

### Identification of Inhibitors Against Steroid Resistance

A process of identifying inhibitors of steroid resistance is provided, including contacting a candidate agent with a population of cells including genetically modified leukocytes that express a gene that confers resistance to 11-beta-hydroxysteroids, and measuring a loss or reduction of resistance to steroids of the genetically modified leukocytes. Candidate agents that impart a measurable loss or reduction of resistance to steroids are identified as an inhibitor.

### Improvement of Growth of Genetically Modified Leukocytes

A method for improving the growth of genetically modified leukocytes is also provided by contacting an effective amount of an inhibitor of HSD activity with cultures of genetically modified leukocytes that express a gene that confers resistance to 11-beta-hydroxysteroids.

Differences from Others Methods

Zhang H, et al., in Biochemical and Biophysical Research Communications 490 (2017) 1399-1406 describe transfecting a gene for HSD11B2 into murine bone-like cells (MLO-Y4) and murine osteoblast-like cells (MC3T3-E1) and exposing them to dexamethasone. Apoptosis was detected by flow cytometry for 7AAD and Annexin V. The authors reported addition of dexamethasone (DEX) increased apoptosis in MC3T3-E1 cells from (approximate numbers) 7% in the control group to 29% in the DEX-treated group, and fell to 14% if the cells were transduced with the gene for HSD2 prior to DEX treatment. The authors reported that addition of DEX increased apoptosis in MLO-Y4 cells demonstrated apoptosis in (approximate numbers) 6% in the control group to 24% in the DEX-treated group, and fell to 12% if the cells were transfected with the gene for HSD11B2. Delivery of the gene for HSD11B2 plus a short interfering RNA (siRNA) against HSD11B2 using an adenoviral system resulted in increased apoptosis when cells were treated with DEX (37% apoptosis for MC3T3-E1 and 25% for MLO-Y4 compared to control arm with apoptosis of 22% and 17%, respectively).

However, the study by Zhang H, et al. used relatively high doses of DEX (100 nM and 1 μM for MC3T3-E1 and MLO-Y4, respectively) to drive what was described as apoptosis. The species of HSD11B2 gene used (e.g. mouse or human) was not stated, but the PCR primers used for real time PCR for mRNA levels were designed against the murine HSD11B2 sequence.

To the best of Applicant's knowledge, prior to the present invention, there are no studies that deliver an external gene to confer steroid resistance on human hematopoietic cells including hematopoietic cell lines.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Cloning and Transfection and Laboratory Work

Genes may be synthesized or obtained from commercial vendors (e.g Origene, Integrated DNA Technologies, Life Technologies, Thermo Fischer, Invitrogen) and cloned into vectors obtained from the same commercial vendors. Such vendors also supply transfection reagents, cell lines, tissue culture media, and reagents to perform analyses of cells and enzyme activity. Chemicals obtained from vendors such as Sigma Aldrich and TCI. Human cell products obtained from approved sources such as the Cell Processing Core at Cincinnati Children's Hospital (CCHMC). Flow cytometry analyses obtained from core laboratories including Research Flow Cytometry Core at CCHMC.

The conferring of glucocorticoid resistance was assessed by methods including: (i) survival in the presence of glucocorticoids, measured by methods including but not limited to: cell counts, cell viability, MTT assay, apoptosis assays (tube or plate-based, imaging, flow cytometry), (ii) using techniques to measure reduction in immune activity, (iii) cells lines that die in the presence of glucocorticoids, (iv) detecting the nuclear translocation of GR, and (v) reporter genes and constructs that ‘report’ glucocorticoid action via GR.

Cloning of HSD Genes into the pCCL Vector

Specifically, lentiviral vector pCCL-c-MNDU3c-X (DB Kohn, University of California Los Angeles, Calif.) (“backbone vector”) was cleaved using a double digest with restriction enzymes Acc65I and EcoRI in NEBuffer 3.1 using an extended incubation time in accordance with manufacturers guidance (New England Biolabs, Ipswich, Mass.). Digested vector was separated by gel electrophoresis and the band corresponding to the linearized cleavage product was visualized, excised and purified (Monarch DNA Gel Extraction Kit, New England Biolabs, Ipswich, Mass.).

Insert gene sequences were codon optimized for gene synthesis and designed with additional 5′ and 3′ sequences which corresponded to the vector sequences adjacent to the predicted Acc65I and EcoRI restriction enzyme cleavage sites in the vector. Genes for human HSD11B1L isoforms were edited to have a common partial Kozak sequence through the first methionine (ATG start) codon. Insert genes were synthesized as GBLOCKS® gene fragments by Integrated DNA Technologies, Coralville, Iowa Inserts were cloned into the double digested lentivector backbone using assembly cloning according to manufacturer's instructions, transformed into NEB 5-alpha competent E. coli (NEBuilder HiFI DNA Assembly Cloning Kit, New England Biolabs, Ipswich, Mass.) and plated on LB agar plates with 100 ug/ml ampicillin (Teknova, Hollister, Calif.). Following overnight incubation at 37° C., individual colonies were picked, cultured in liquid broth and vector DNA purified. DNA was sequenced using forward primer, CCAAGGACCTGAAATGACCC (SEQ ID NO: 15), and reverse primer, CTGAATAATAAGATGACATGAACTACTACTGC (SEQ ID NO: 16) (Functional Biosciences, Madison, Wis.). Sequencing using these primers produced reads that span the vector-insert junction and produce overlapping reads. Selected clones that incorporated a full-length insert sequence in the anticipated site in the lenti-vector were produced at the “maxi-prep” scale (1 mg) and purified (Plasmid.com, Fargo, N. Dak.).

The HSD synthetic DNAs (DNA sequence of the GBLOCKS®) had 5′ and 3′ extensions to match the vector and are codon optimized for the DNA synthesis process at IDT. Further, the HSD11B1L genes (of various isoforms) have been designed with a uniform (partial) Kozak consensus sequence (up to the first ATG/Met). Therefore, the constructs are longer than the reference DNA coding sequences, but they encode the same proteins as the protein sequence encoded by their respective reference DNA coding sequences. (Protein sequences that are known and accessible from public database are used as reference protein sequences: e.g., SEQ ID Nos: 3-14) Specifically, Applicant's genes typically have a general structure of:

vector sequence-Kozak-ATG-gene-stop codon-vector sequence.

For clarity, Kozak sequences overlap with start ATG (Met) codon and the first base of the codon following the ATG. The GBLOCK®s have the following identifiable features (written 5′ to 3′; hyphens used for clarifying punctuation only) for HSD11B1L multiple human genes: region homologous with lenti vector-5′ untranslated region (UTR) and region of Kozak from the HSD11B1L isoform b with ATG start codon+rest of codon optimized gene and stop codon-EcoNI site (if the coding sequence does not already have one)-Acc65I Site-region homologous to vector. The GBLOCK®s have the following identifiable features (written 5′ to 3′; hyphens used for clarifying punctuation only) for the HSD11B1L chimp gene: the HSD11B1Lchimp (encodes a 286 amino acid protein) GBLOCK® comprises the GBLOCK® for the human HSD11B1L encoding the 286 amino acid HSD11B1L (human isoform/variant b) where a codon optimized chimpanzee coding sequence replaces the human coding sequence. For HSD11B1 and HSD11B2, the HSD11B1 GBLOCK® and the HSD11B2 GBLOCK® include: region homologous with lenti vector-5′ UTR region-Kozak sequence from each HSD gene with ATG start codon-rest of codon optimized gene and stop codon-EcoNI site (if the coding sequence does not already have one)-Acc65I Site-region homologous to vector. The SER6mod GBLOCK® is organized: region homologous with lenti vector-5′ UTR region-Kozak with ATG start codon-rest of codon optimized modified gene (this sequence omits the natural leader sequence) and stop codon-EcoNI site-Acc65I Site-region homologous to vector.

The following DNA sequences are indicated by a denotation of “Name,” “type” and “Sequence Length in base pairs (bp)”. GBLOCK® (gBlocks Gene Fragments) are sequence-verified, double-stranded DNA fragments custom-made and provided by Integrated DNA Technologies.

HSD11B1 GBLOCK ® (970 bp):
(SEQ ID NO: 17)
CCCCTCACTCGGCGCGATCTAGATCTCGAATCGCCCTGTCGGATGGCTTT
TATGAAAAAATATCTCCTCCCCATTCTGGGGCTCTTCATGGCCTACTACT
ACTATTCTGCAAACGAGGAATTCAGACCAGAGATGCTCCAAGGAAAGAAA
GTGATTGTCACAGGGGCCAGCAAAGGGATCGGAAGAGAGATGGCTTATCA
TCTGGCGAAGATGGGAGCCCATGTGGTGGTGACAGCGAGGTCAAAAGAAA
CTCTACAGAAGGTGGTATCCCACTGCCTGGAGCTTGGAGCAGCCTCAGCA
CACTACATTGCTGGCACCATGGAAGACATGACCTTCGCAGAGCAATTTGT
TGCCCAAGCAGGAAAGCTCATGGGAGGACTAGACATGCTCATTCTCAACC
ACATCACCAACACTTCTTTGAATCTTTTTCATGATGATATTCACCATGTG
CGCAAAAGCATGGAAGTCAACTTCCTCAGTTACGTGGTCCTGACTGTAGC
TGCCTTGCCCATGCTGAAGCAGAGCAATGGAAGCATTGTTGTCGTCTCCT
CTCTGGCTGGGAAAGTGGCTTATCCAATGGTTGCTGCCTATTCTGCAAGC
AAGTTTGCTTTGGATGGGTTCTTCTCCTCCATCAGAAAGGAATATTCAGT
GTCCAGGGTCAATGTATCAATCACTCTCTGTGTTCTTGGCCTCATAGACA
CAGAAACAGCCATGAAGGCAGTTTCTGGGATAGTCCATATGCAAGCAGCT
CCAAAGGAGGAATGTGCCCTGGAGATCATCAAAGGGGGAGCTCTGCGCCA
AGAAGAAGTGTATTATGACAGCTCACTCTGGACCACTCTTCTGATCAGAA
ATCCATGCAGGAAGATCCTGGAATTTCTCTACTCAACGAGCTATAATATG
GACAGATTCATAAACAAGTAGCCTGAAAAAGGGGTACCTTTAAGACCAAT
GACTTACAAGGCAGCTGTAG.
HSD11B2 GBLOCK ® (1,318 bp):
(SEQ ID NO: 18)
CCCCTCACTCGGCGCGATCTAGATCTCGAATCGCCAGCCCGCTGGGCCGC
CATGGAGCGTTGGCCTTGGCCATCGGGTGGTGCTTGGCTGCTCGTGGCTG
CTCGTGCACTGCTGCAGCTGCTGCGTTCAGACCTGCGTCTGGGTCGTCCA
CTGCTGGCAGCACTGGCACTGCTGGCTGCACTCGACTGGCTGTGCCAGCG
TCTGCTGCCTCCACCAGCTGCACTCGCTGTGCTGGCTGCTGCTGGTTGGA
TCGCATTGTCCCGTCTGGCACGTCCACAGCGTCTGCCAGTGGCTACTCGT
GCAGTGCTCATCACCGGTTGTGACTCTGGTTTTGGTAAGGAGACGGCTAA
GAAACTGGACTCCATGGGTTTCACGGTGCTGGCTACCGTATTGGAGTTGA
ACAGCCCTGGTGCTATCGAGCTGCGTACCTGCTGCTCCCCTCGTCTAAGG
CTGCTGCAGATGGACCTGACCAAACCAGGAGACATTAGCCGTGTGCTAGA
GTTCACCAAGGCTCACACCACCAGCACCGGTCTGTGGGGTCTCGTCAACA
ACGCAGGTCACAATGAAGTAGTTGCTGATGCAGAGCTGTCTCCAGTGGCT
ACTTTCCGTAGCTGCATGGAGGTGAATTTCTTTGGTGCACTCGAGCTGAC
CAAGGGTCTCCTGCCTCTGCTGCGTAGCTCAAGGGGTCGTATCGTGACTG
TGGGAAGCCCAGCAGGAGACATGCCATATCCATGCTTGGGAGCTTATGGA
ACCTCCAAAGCAGCTGTGGCACTACTCATGGACACATTCAGCTGTGAACT
CCTTCCTTGGGGAGTCAAGGTCAGCATCATCCAGCCTGGTTGCTTCAAGA
CAGAGTCAGTGAGAAACGTGGGTCAGTGGGAAAAGCGTAAGCAATTGCTG
CTGGCTAACCTGCCTCAAGAGCTGCTGCAGGCTTACGGTAAGGACTACAT
CGAGCACTTGCATGGACAGTTCCTGCACTCGCTACGTCTGGCTATGTCCG
ACCTCACCCCAGTTGTAGATGCTATCACAGATGCACTGCTGGCAGCTAGG
CCTCGTCGTCGTTATTACCCTGGTCAGGGTCTGGGACTCATGTACTTCAT
CCACTACTACCTGCCTGAAGGTCTGAGGCGTCGTTTCCTGCAGGCTTTCT
TCATCAGTCACTGTCTGCCTCGAGCACTGCAGCCTGGTCAGCCTGGTACT
ACCCCACCACAGGACGCAGCTCAGGACCCAAACCTGAGCCCTGGTCCTTC
CCCAGCAGTGGCTAGGTGACCTGAAAAAGGGGTACCTTTAAGACCAATGA
CTTACAAGGCAGCTGTAG.
HSD11B1Lchimp GBLOCK ® (938 bp):
(SEQ ID NO: 19)
CCCCTCACTCGGCGCGATCTAGATCTCGAATCGAGGACCATGAAGGTGCT
TCTCCTCACAGGACTGGGAGCTCTGTTCTTCGCTTATTATTGGGATGACA
ACTTTGACCCAGCTAGCCTCCAGGGAGCACGAGTGCTGCTGACAGGAGCT
AATGCTGGTGTTGGTGAGGAGCTGGCTTATCACTACGCACGTCTGGGTTC
CCACCTGGTGCTCACTGCTCACACTGAGGCTCTCCTGCAGAAGGTGGTAG
GAAACTGCAGGAAGCTGGGTGCTCCTAAGGTCTTCTACATCGCAGCAGAC
ATGGCTTCCCCTGAGGCACCTGAGAGCGTGGTGCAGTTTGCACTGGACAA
GCTGGGTGAGGGACTGGGTCTGAATCCTGGAGTCAGGGACCGTGGTCTAG
GTCTTAGGGACAGGACCAGAATTGGACTGTGGTGCCGTCTGCAGGTAAAC
TTTGTGAGCTACGTGCAACTGACGTCGAGGGCACTGCCTAGCCTGACAGA
CAGCAAGGGTTCCCTGGTGGTGGTGTCCTCGCTGCTCGGTCGTGTGCCTA
CGTCGTTCTCCACTCCATACTCGGCAGCTAAGTTTGCACTGGACGGTTTC
TTCGGTTCCCTGAGGAGGGAGCTGGACGTGCAGGACGTGAACGTGGCTAT
CACCATGTGCGTCCTGGGTCTCCGAGATCGTGCTTCCGCTGCTGAGGCAG
TCAGGGGAGTCACGAGGGTCAAGGCAGCTCCAGGACCTAAGGCAGCTCTG
GCTGTGATCCGTGGTGGTGCTACGCGTGCAGCTGGTGTCTTCTACCCATG
GCGTTTCCGTCTGCTGTGCTTGCTCAGGCGTTGGCTGCCACGTCCAAGGG
CTTGGTTTATCCGTCAGGAGCTCAACGTCACGGCTGCAGCTGCAGCTTGA
GGTACCTTTAAGACCAATGACTTACAAGGCAGCTGTAG.
HSD11B1Lg GOLF GBLOCK (1,079 bp):
(SEQ ID NO: 20)
CCCCTCACTCGGCGCGATCTAGATCTCGAATCGAGGACCATGGCAAATCT
CGGTACACTACAACTTCTGCCTCCTAGGTTCAAGCGATTCTCCTGCCTCA
GCCTCCCAAATATCTGGATTACAGGTATGCCTGTGCCAGCTACCTCTGTC
CCTTGTCCTTCTGCAGGTCCACACAGGACCATGAAGGTGCTTCTCCTCAC
AGGACTGGGAGCTCTGTTCTTCGCTTATTATTGGGATGACAACTTCGACC
CAGCTAGCCTCCAGGGAGCACGAGTGCTGCTGACAGGAGCTAACGCTGGT
GTTGGTGAGGAGCTGGCTTATCACTACGCACGTCTGGGTTCCCACCTGGT
GCTCACTGCTCACACTGAGGCTCTCCTGCAGAAGGTGGTAGGAAACTGCA
GGAAGCTGGGTGCTCCTAAGGTCTTCTACATCGCAGCAGACATGGCTTCC
CCTGAGGCACCTGAGAGCGTGGTGCAGTTTGCACTGGACAAGCTGGGTGG
ACTGGACTACCTCGTGCTGAACCACATCGGTGGTGCTCCAGCTGGTACGC
GAGCTCGTAGCCCTCAGGCAACTCGTTGGCTCATGCAGGTAAACTTTGTG
AGCTACGTGCAACTGACGTCGAGGGCACTGCCTAGCCTGACGGACAGCAA
GGGTTCCCTGGTGGTGGTGTCCTCGCTGCTCGGTCGTGTGCCTACGTCGT
TCTCCACTCCTTACTCGGCAGCTAAGTTTGCACTGGACGGTTTCTTCGGT
TCCCTGAGGAGGGAGCTGGACGTGCAGGACGTGAACGTGGCTATCACCAT
GTGCGTCCTGGGTCTCCGAGATCGTGCTTCCGCTGCTGAGGCAGTCAGGG
GAGTCACGAGGGTCAAGGCAGCTCCAGGACCTAAGGCAGCTCTGGCTGTG
ATCCGTGGTGGTGCTACGCGTGCAGCTGGTGTCTTCTACCCATGGCGTTT
CCGTCTGCTGTGCTTGCTCAGGCGTTGGCTACCACGTCCAAGGGCTTGGT
TTATCCGTCAGGAGCTCAACGTCACGGCTGCAGCAGCTTGAGGTACCTTT
AAGACCAATGACTTACAAGGCAGCTGTAG.
HSD11B1Lb BRAVO GBLOCK ® (938 bp):
(SEQ ID NO: 21)
CCCCTCACTCGGCGCGATCTAGATCTCGAATCGAGGACCATGAAGGTGCT
TCTCCTCACAGGACTGGGAGCTCTGTTCTTCGCTTATTATTGGGATGACA
ACTTCGACCCAGCTAGCCTCCAGGGAGCACGAGTGCTGCTGACAGGAGCT
AACGCTGGTGTTGGTGAGGAGCTGGCTTATCACTACGCACGTCTGGGTTC
CCACCTGGTGCTCACTGCTCACACTGAGGCTCTCCTGCAGAAGGTGGTAG
GAAACTGCAGGAAGCTGGGTGCTCCTAAGGTCTTCTACATCGCAGCAGAC
ATGGCTTCCCCTGAGGCACCTGAGAGCGTGGTGCAGTTTGCACTGGACAA
GCTGGGTGGACTGGACTACCTCGTGCTGAACCACATCGGTGGTGCTCCAG
CTGGTACGCGAGCTCGTAGCCCTCAGGCAACTCGTTGGCTCATGCAGGTA
AACTTTGTGAGCTACGTGCAACTGACGTCGAGGGCACTGCCTAGCCTGAC
GGACAGCAAGGGTTCCCTGGTGGTGGTGTCCTCGCTGCTCGGTCGTGTGC
CTACGTCGTTCTCCACTCCTTACTCGGCAGCTAAGTTTGCACTGGACGGT
TTCTTCGGTTCCCTGAGGAGGGAGCTGGACGTGCAGGACGTGAACGTGGC
TATCACCATGTGCGTCCTGGGTCTCCGAGATCGTGCTTCCGCTGCTGAGG
CAGTCAGGGGAGTCACGAGGGTCAAGGCAGCTCCAGGACCTAAGGCAGCT
CTGGCTGTGATCCGTGGTGGTGCTACGCGTGCAGCTGGTGTCTTCTACCC
ATGGCGTTTCCGTCTGCTGTGCTTGCTCAGGCGTTGGCTACCACGTCCAA
GGGCTTGGTTTATCCGTCAGGAGCTCAACGTCACGGCTGCAGCAGCTTGA
GGTACCTTTAAGACCAATGACTTACAAGGCAGCTGTAG
HSD11B1Lc CHARLIE GBLOCK (688 bp):
(SEQ ID NO: 22)
CCCCTCACTCGGCGCGATCTAGATCTCGAATCGAGGACCATGGCTTCCCC
TGAGGCACCTGAGAGCGTGGTGCAGTTTGCACTGGACAAGCTGGGTGGAC
TGGACTACCTCGTGCTGAACCACATCGGTGGTGCTCCAGCTGGTACGCGA
GCTCGTAGCCCTCAGGCAACTCGTTGGCTCATGCAGGTAAACTTTGTGAG
CTACGTGCAACTGACGTCGAGGGCACTGCCTAGCCTGACGGACAGCAAGG
GTTCCCTGGTGGTGGTGTCCTCGCTGCTCGGTCGTGTGCCTACGTCGTTC
TCCACTCCTTACTCGGCAGCTAAGTTTGCACTGGACGGTTTCTTCGGTTC
CCTGAGGAGGGAGCTGGACGTGCAGGACGTGAACGTGGCTATCACCATGT
GCGTCCTGGGTCTCCGAGATCGTGCTTCCGCTGCTGAGGCAGTCAGGGGA
GTCACGAGGGTCAAGGCAGCTCCAGGACCTAAGGCAGCTCTGGCTGTGAT
CCGTGGTGGTGCTACGCGTGCAGCTGGTGTCTTCTACCCATGGCGTTTCC
GTCTGCTGTGCTTGCTCAGGCGTTGGCTACCACGTCCAAGGGCTTGGTTT
ATCCGTCAGGAGCTCAACGTCACGGCTGCAGCAGCTTGACCTGAAAAAGG
GGTACCTTTAAGACCAATGACTTACAAGGCAGCTGTAG.
HSD11B1Ld DELTA GBLOCK ® (547 bp):
(SEQ ID NO: 23)
CCCCTCACTCGGCGCGATCTAGATCTCGAATCGAGGACCATGCAGGTAAA
CTTTGTGAGCTACGTGCAACTGACGTCGAGGGCACTGCCTAGCCTGACGG
ACAGCAAGGGTTCCCTGGTGGTGGTGTCCTCGCTGCTCGGTCGTGTGCCT
ACGTCGTTCTCCACTCCTTACTCGGCAGCTAAGTTTGCACTGGACGGTTT
CTTCGGTTCCCTGAGGAGGGAGCTGGACGTGCAGGACGTGAACGTGGCTA
TCACCATGTGCGTCCTGGGTCTCCGAGATCGTGCTTCCGCTGCTGAGGCA
GTCAGGGGAGTCACGAGGGTCAAGGCAGCTCCAGGACCTAAGGCAGCTCT
GGCTGTGATCCGTGGTGGTGCTACGCGTGCAGCTGGTGTCTTCTACCCAT
GGCGTTTCCGTCTGCTGTGCTTGCTCAGGCGTTGGCTACCACGTCCAAGG
GCTTGGTTTATCCGTCAGGAGCTCAACGTCACGGCTGCAGCAGCTTGACC
TGAAAAAGGGGTACCTTTAAGACCAATGACTTACAAGGCAGCTGTAG.
HSD11B1Le ECHO GBLOCK ® (1,025 bp):
(SEQ ID NO: 24)
CCCCTCACTCGGCGCGATCTAGATCTCGAATCGAGGACCATGAAGGTGCT
TCTCCTCACAGGACTGGGAGCTCTGTTCTTCGCTTATTATTGGGATGACA
ACTTCGACCCAGCTAGCCTCCAGGGAGCACGAGTGCTGCTGACAGGAGCT
AACGCTGGTGTTGGTGAGGAGCTGGCTTATCACTACGCACGTCTGGGTTC
CCACCTGGTGCTCACTGCTCACACTGAGGCTCTCCTGCAGAAGGTGGTAG
GAAACTGCAGGAAGCTGGGTGCTCCTAAGGTCTTCTACATCGCAGCAGAC
ATGGCTTCCCCTGAGGCACCTGAGAGCGTGGTGCAGTTTGCACTGGACAA
GCTGGGTGGACTGGACTACCTCGTGCTGAACCACATCGGTGGTGCTCCAG
CTGGTACGCGAGCTCGTAGCCCTCAGGCAACTCGTTGGCTCATGCAGGTA
AACTTTGTGAGCTACGTGCAACTGACGTCGAGGGCACTGCCTAGCCTGAC
GGACAGCAAGGGTTCCCTGGTGGTGGTGTCCTCGCTGCTCGGTCGTGTGC
CTACGTCGTTCTCCACTCCTTACTCGGCAGCTAAGTTTGCACTGGACGGT
TTCTTCGGTTCCCTGAGGAGGGAGCTGGACGTGCAGGACGTGAACGTGGC
TATCACCATGTGCGTCCTGGGTCTCCGAGATCGTGCTTCCGCTGCTGAGG
CAGTCAGGAGCTCAACGTCAAGGCCAAGGCAGCCTGAGCACAGGGGAGTG
CCTCTCCAGTCCCAGACGGCAATGTTCCTCCCTCCAACTGTCCCTGGAGC
TAGAACACTCACAGAGACACCTCTGAGAGGATGGCCACAGCCTAAGATGA
AGTCATCAAGACAGAAAAGCAAAACCGAGAAAAACGACGGACACCTGGAA
CCAGTCACGGCTTGGGAGGTGCAGGTGCCTCGTGTTAGGCGTCTTTGTAG
GGGACTTGCAAGGCCTCACCTGTTTGGTCATGATTGAGGTACCTTTAAGA
CCAATGACTTACAAGGCAGCTGTAG.
HSD11B1La ALPHA GBLOCK ® (706 bp):
(SEQ ID NO: 25)
CCCCTCACTCGGCGCGATCTAGATCTCGAATCGAGGACCATGAAGGTGCT
TCTCCTCACAGGACTGGGAGCTCTGTTCTTCGCTTATTATTGGGATGACA
ACTTCGACCCAGGTGGACTGGACTACCTCGTGCTGAACCACATCGGTGGT
GCTCCAGCTGGTACGCGAGCTCGTAGCCCTCAGGCAACTCGTTGGCTCAT
GCAGGTAAACTTTGTGAGCTACGTGCAACTGACGTCGAGGGCACTGCCTA
GCCTGACGGACAGCAAGGGTTCCCTGGTGGTGGTGTCCTCGCTGCTCGGT
CGTGTGCCTACGTCGTTCTCCACTCCTTACTCGGCAGCTAAGTTTGCACT
GGACGGTTTCTTCGGTTCCCTGAGGAGGGAGCTGGACGTGCAGGACGTGA
ACGTGGCTATCACCATGTGCGTCCTGGGTCTCCGAGATCGTGCTTCCGCT
GCTGAGGCAGTCAGGGGAGTCACGAGGGTCAAGGCAGCTCCAGGACCTAA
GGCAGCTCTGGCTGTGATCCGTGGTGGTGCTACGCGTGCAGCTGGTGTCT
TCTACCCATGGCGTTTCCGTCTGCTGTGCTTGCTCAGGCGTTGGCTACCA
CGTCCAAGGGCTTGGTTTATCCGTCAGGAGCTCAACGTCACGGCTGCAGC
AGCTTGACCTGAAAAAGGGGTACCTTTAAGACCAATGACTTACAAGGCAG
CTGTAG.
HSD11B1Lh HOTEL GBLOCK ® (692 bp):
(SEQ ID NO: 26)
CCCCTCACTCGGCGCGATCTAGATCTCGAATCGAGGACCATGAAGGTGCT
TCTCCTCACAGGACTGGGAGCTCTGTTCTTCGCTTATTATTGGGATGACA
ACTTCGACCCAGGTAAACTTTGTGAGCTACGTGCAACTGACGTCGCAGGT
GCTGCTCAGCCTGACGGACAGCAAGGACTCCCTGGTGGTGGTGTCCTCGC
TGCTAGGCCACGTGCTCACGTCGTTCTCCACTCCCTACTCGGTGGTCAAG
TTTGCGCTGGAAGGCTTCTTAGGCTCCCTGCAGCAGGAGCTGGACGTGCA
GGACGTGAACGTGGTCATCACCATGTGCGTCCTGGACCTCCAAGATCGCG
TCTCCGTCGTCGAGGTAGTCAGGGAAGTCACGAGGGTCAAGGTGGTCCTG
GAGCTCAAGGTAGCCCTGGTCGTGATCCAAGGAGGCGTCACGCACGTGGT
AGGCGTCTTCTACCTGTGGCATTTCCACCTGCTGTGCTTGCTCCAGCACT
GGCTACCGCACCTGCAGGTCTGGTTTATCCACCAGGAGCTCAACGTCACG
GTCGTGGTAGCCTGAGCACCGGAGGATGCCCTTCCAGTCCTAGAAGGCAA
TGTTCCTCCCTCCAACTGTCCCTGGAGCCAGAACACTCACAGAGACACCC
TTGAGGTACCTTTAAGACCAATGACTTACAAGGCAGCTGTAG.
HSD11B1Lf FRANK GBLOCK ® (623 bp):
(SEQ ID NO: 27)
CCCCTCACTCGGCGCGATCTAGATCTCGAATCGAGGACCATGCAGGTAAA
CTTTGTGAGCTACGTGCAACTGACGTCGAGGGCACTGCCTAGCCTGACGG
ACAGCAAGGGTTCCCTGGTGGTGGTGTCCTCGCTGCTCGGTCGTGTGCCT
ACGTCGTTCTCCACTCCTTACTCGGCAGCTAAGTTTGCACTGGACGGTTT
CTTCGGTTCCCTGAGGAGGGAGCTGGACGTGCAGGACGTGAACGTGGCTA
TCACCATGTGCGTCCTGGGTCTCCGAGATCGTGCTTCCGCTGCTGAGGCA
GTCAGGAGCTCAACGTCAAGGCCAAGGCAGCCTGAGCACAGGGGAGTGCC
TCTCCAGTCCCAGACGGCAATGTTCCTCCCTCCAACTGTCCCTGGAGCTA
GAACACTCACAGAGACACCTCTGAGAGGATGGCCACAGCCTAAGATGAAG
TCATCAAGACAGAAAAGCAAAACCGAGAAAAACGACGGACACCTGGAACC
AGTCACGGCTTGGGAGGTGCAGGTGCCTCGTGTTAGGCGTCTTTGTAGGG
GACTTGCAAGGCCTCACCTGTTTGGTCATGATTGAGGTACCTTTAAGACC
AATGACTTACAAGGCAGCTGTAG.
HSD11B1Li INDIA GBLOCK ® (782 bp):
(SEQ ID NO: 28)
CCCCTCACTCGGCGCGATCTAGATCTCGAATCGAGGACCATGAAGGTGCT
TCTCCTCACAGGACTGGGAGCTCTGTTCTTCGCTTATTATTGGGATGACA
ACTTCGACCCAGGTGGACTGGACTACCTCGTGCTGAACCACATCGGTGGT
GCTCCAGCTGGTACGCGAGCTCGTAGCCCTCAGGCAACTCGTTGGCTCAT
GCAGGTAAACTTTGTGAGCTACGTGCAACTGACGTCGAGGGCACTGCCTA
GCCTGACGGACAGCAAGGGTTCCCTGGTGGTGGTGTCCTCGCTGCTCGGT
CGTGTGCCTACGTCGTTCTCCACTCCTTACTCGGCAGCTAAGTTTGCACT
GGACGGTTTCTTCGGTTCCCTGAGGAGGGAGCTGGACGTGCAGGACGTGA
ACGTGGCTATCACCATGTGCGTCCTGGGTCTCCGAGATCGTGCTTCCGCT
GCTGAGGCAGTCAGGAGCTCAACGTCAAGGCCAAGGCAGCCTGAGCACAG
GGGAGTGCCTCTCCAGTCCCAGACGGCAATGTTCCTCCCTCCAACTGTCC
CTGGAGCTAGAACACTCACAGAGACACCTCTGAGAGGATGGCCACAGCCT
AAGATGAAGTCATCAAGACAGAAAAGCAAAACCGAGAAAAACGACGGACA
CCTGGAACCAGTCACGGCTTGGGAGGTGCAGGTGCCTCGTGTTAGGCGTC
TTTGTAGGGGACTTGCAAGGCCTCACCTGTTTGGTCATGATTGAGGTACC
TTTAAGACCAATGACTTACAAGGCAGCTGTAG.
SER6mod GBLOCK ® (1,246 bp):
(SEQ ID NO: 29)
CCCCTCACTCGGCGCGATCTAGATCTCGAATCGCTATACTGGACAATGGA
TCCTAACGCTGCTTATGTGAACATGAGTAACCATCACAGGGGTCTGGCTT
CAGCTAACGTTGACTTTGCTTTCAGCCTGTATAAGCACCTAGTGGCTTTG
AGTCCTAAAAAGAACATTTTCATCTCCCCTGTGAGCATCTCCATGGCTTT
AGCTATGCTGTCCCTGGGTACCTGTGGTCACACAAGGGCTCAGCTTCTCC
AGGGTCTGGGTTTCAACCTCACTGAGAGGTCTGAGACTGAGATCCACCAG
GGTTTCCAGCACCTGCACCAACTCTTTGCAAAGTCAGACACCAGCTTAGA
AATGACCATGGGTAATGCTTTGTTTCTTGATGGTAGCCTGGAGTTGCTGG
AGTCATTCTCAGCAGACATCAAGCACTACTATGAGTCAGAGGTCTTGGCT
ATGAATTTCCAGGACTGGGCAACAGCTAGCAGACAGATCAACAGCTATGT
CAAGAATAAGACACAGGGAAAAATTGTCGACTTGTTTTCAGGACTGGATA
GCCCAGCTATCCTCGTCCTGGTCAACTATATCTTCTTCAAAGGTACATGG
ACACAGCCTTTTGACCTGGCAAGCACCAGGGAGGAGAACTTCTATGTGGA
CGAGACAACTGTGGTGAAGGTGCCTATGATGTTGCAGTCGAGCACCATCA
GTTACCTTCATGACGCAGAGCTCCCTTGCCAGCTGGTGCAGATGAACTAC
GTGGGTAATGGAACTGTCTTCTTCATCCTTCCAGACAAGGGAAAGATGAA
CACAGTCATCGCTGCACTGAGCAGGGACACGATTAACAGGTGGTCCGCAG
GTCTGACCAGCAGCCAGGTGGACCTGTACATTCCAAAGGTCACCATCTCT
GGAGTCTATGACCTCGGAGATGTGCTGGAGGAAATGGGTATTGCAGACTT
GTTCACCAACCAGGCAAATTTCTCACGTATCACCCAGGACGCTCAGCTGA
AGTCATCAAAGGTGGTCCATAAAGCTGTGCTGCAACTCAATGAGGAGGGT
GTGGACACAGCTGGTTCCACTGGAGTCACCCTAAACCTGACGTCCAAGCC
TATCATCTTGCGTTTCAACCAGCCTTTCATCATCATGATCTTCGACCACT
TCACCTGGAGCAGCCTTTTCCTGGCAAGGGTTATGAACCCAGTGTAACCT
GAAAAAGGGGTACCTTTAAGACCAATGACTTACAAGGCAGCTGTAG.
The backbone vector pCCL-c-MNDU3c-X
sequence contains 6,571 bps:
(SEQ ID NO: 30)
CAGGTGGCACTTTTCGGGGAAATGTGCGCGGAACCCCTATTTGTTTATTT
TTCTAAATACATTCAAATATGTATCCGCTCATGAGACAATAACCCTGATA
AATGCTTCAATAATATTGAAAAAGGAAGAGTATGAGTATTCAACATTTCC
GTGTCGCCCTTATTCCCTTTTTTGCGGCATTTTGCCTTCCTGTTTTTGCT
CACCCAGAAACGCTGGTGAAAGTAAAAGATGCTGAAGATCAGTTGGGTGC
ACGAGTGGGTTACATCGAACTGGATCTCAACAGCGGTAAGATCCTTGAGA
GTTTTCGCCCCGAAGAACGTTTTCCAATGATGAGCACTTTTAAAGTTCTG
CTATGTGGCGCGGTATTATCCCGTATTGACGCCGGGCAAGAGCAACTCGG
TCGCCGCATACACTATTCTCAGAATGACTTGGTTGAGTACTCACCAGTCA
CAGAAAAGCATCTTACGGATGGCATGACAGTAAGAGAATTATGCAGTGCT
GCCATAACCATGAGTGATAACACTGCGGCCAACTTACTTCTGACAACGAT
CGGAGGACCGAAGGAGCTAACCGCTTTTTTGCACAACATGGGGGATCATG
TAACTCGCCTTGATCGTTGGGAACCGGAGCTGAATGAAGCCATACCAAAC
GACGAGCGTGACACCACGATGCCTGTAGCAATGGCAACAACGTTGCGCAA
ACTATTAACTGGCGAACTACTTACTCTAGCTTCCCGGCAACAATTAATAG
ACTGGATGGAGGCGGATAAAGTTGCAGGACCACTTCTGCGCTCGGCCCTT
CCGGCTGGCTGGTTTATTGCTGATAAATCTGGAGCCGGTGAGCGTGGGTC
TCGCGGTATCATTGCAGCACTGGGGCCAGATGGTAAGCCCTCCCGTATCG
TAGTTATCTACACGACGGGGAGTCAGGCAACTATGGATGAACGAAATAGA
CAGATCGCTGAGATAGGTGCCTCACTGATTAAGCATTGGTAACTGTCAGA
CCAAGTTTACTCATATATACTTTAGATTGATTTAAAACTTCATTTTTAAT
TTAAAAGGATCTAGGTGAAGATCCTTTTTGATAATCTCATGACCAAAATC
CCTTAACGTGAGTTTTCGTTCCACTGAGCGTCAGACCCCGTAGAAAAGAT
CAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAG
CTACCAACTCTTTTTCCGAAGGTAACTGGCTTCAGCAGAGCGCAGATACC
AAATACTGTCCTTCTAGTGTAGCCGTAGTTAGGCCACCACTTCAAGAACT
CTGTAGCACCGCCTACATACCTCGCTCTGCTAATCCTGTTACCAGTGGCT
GCTGCCAGTGGCGATAAGTCGTGTCTTACCGGGTTGGACTCAAGACGATA
GTTACCGGATAAGGCGCAGCGGTCGGGCTGAACGGGGGGTTCGTGCACAC
AGCCCAGCTTGGAGCGAACGACCTACACCGAACTGAGATACCTACAGCGT
GAGCTATGAGAAAGCGCCACGCTTCCCGAAGGGAGAAAGGCGGACAGGTA
TCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAA
AAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGT
ATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAAC
CGCCTCTCCCCGCGCGTTGGCCGATTCATTAATGCAGCTGGCACGACAGG
TTTCCCGACTGGAAAGCGGGCAGTGAGCGCAACGCAATTAATGTGAGTTA
GCTCACTCATTAGGCACCCCAGGCTTTACACTTTATGCTTCCGGCTCGTA
TGTTGTGTGGAATTGTGAGCGGATAACAATTTCACACAGGAAACAGCTAT
GACCATGATTACGCCAAGCGCGCAATTAACCCTCACTAAAGGGAACAAAA
GCTGGAGCTGCAAGCTTGGCCATTGCATACGTTGTATCCATATCATAATA
TGTACATTTATATTGGCTCATGTCCAACATTACCGCCATGTTGACATTGA
TTATTGACTAGTTATTAATAGTAATCAATTACGGGGTCATTAGTTCATAG
CCCATATATGGAGTTCCGCGTTACATAACTTACGGTAAATGGCCCGCCTG
GCTGACCGCCCAACGACCCCCGCCCATTGACGTCAATAATGACGTATGTT
CCCATAGTAACGCCAATAGGGACTTTCCATTGACGTCAATGGGTGGAGTA
TTTACGGTAAACTGCCCACTTGGCAGTACATCAAGTGTATCATATGCCAA
GTACGCCCCCTATTGACGTCAATGACGGTAAATGGCCCGCCTGGCATTAT
GCCCAGTACATGACCTTATGGGACTTTCCTACTTGGCAGTACATCTACGT
ATTAGTCATCGCTATTACCATGGTGATGCGGTTTTGGCAGTACATCAATG
GGCGTGGATAGCGGTTTGACTCACGGGGATTTCCAAGTCTCCACCCCATT
GACGTCAATGGGAGTTTGTTTTGGCACCAAAATCAACGGGACTTTCCAAA
ATGTCGTAACAACTCCGCCCCATTGACGCAAATGGGCGGTAGGCGTGTAC
GGTGGGAGGTCTATATAAGCAGAGCTCGTTTAGTGAACCGGGGTCTCTCT
GGTTAGACCAGATCTGAGCCTGGGAGCTCTCTGGCTAACTAGGGAACCCA
CTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGT
CAGTGTGGAAAATCTCTAGCAGTGGCGCCCGAACAGGGACCTGAAAGCGA
AAGGGAAACCAGAGGAGCTCTCTCGACGCAGGACTCGGCTTGCTGAAGCG
CGCACGGCAAGAGGCGAGGGGCGGCGACTGGTGAGTACGCCAAAAATTTT
GACTAGCGGAGGCTAGAAGGAGAGAGATGGGTGCGAGAGCGTCAGTATTA
AGCGGGGGAGAATTAGATCGCGATGGGAAAAAATTCGGTTAAGGCCAGGG
GGAAAGAAAAAATATAAATTAAAACATATAGTATGGGCAAGCAGGGAGCT
AGAACGATTCGCAGTTAATCCTGGCCTGTTAGAAACATCAGAAGGCTGTA
GACAAATACTGGGACAGCTACAACCATCCCTTCAGACAGGATCAGAAGAA
CTTAGATCATTATATAATACAGTAGCAACCCTCTATTGTGTGCATCAAAG
GATAGAGATAAAAGACACCAAGGAAGCTTTAGACAAGATAGAGGAAGAGC
AAAACAAAAGTAAGACCACCGCACAGCAAGCGGCCGCTGATCTTCAGACC
TGGAGGAGGAGATATGAGGGACAATTGGAGAAGTGAATTATATAAATATA
AAGTAGTAAAAATTGAACCATTAGGAGTAGCACCCACCAAGGCAAAGAGA
AGAGTGGTGCAGAGAGAAAAAAGAGCAGTGGGAATAGGAGCTTTGTTCCT
TGGGTTCTTGGGAGCAGCAGGAAGCACTATGGGCGCAGCCTCAATGACGC
TGACGGTACAGGCCAGACAATTATTGTCTGGTATAGTGCAGCAGCAGAAC
AATTTGCTGAGGGCTATTGAGGCGCAACAGCATCTGTTGCAACTCACAGT
CTGGGGCATCAAGCAGCTCCAGGCAAGAATCCTGGCTGTGGAAAGATACC
TAAAGGATCAACAGCTCCTGGGGATTTGGGGTTGCTCTGGAAAACTCATT
TGCACCACTGCTGTGCCTTGGAATGCTAGTTGGAGTAATAAATCTCTGGA
ACAGATTGGAATCACACGACCTGGATGGAGTGGGACAGAGAAATTAACAA
TTACACAAGCTTAATACACTCCTTAATTGAAGAATCGCAAAACCAGCAAG
AAAAGAATGAACAAGAATTATTGGAATTAGATAAATGGGCAAGTTTGTGG
AATTGGTTTAACATAACAAATTGGCTGTGGTATATAAAATTATTCATAAT
GATAGTAGGAGGCTTGGTAGGTTTAAGAATAGTTTTTGCTGTACTTTCTA
TAGTGAATAGAGTTAGGCAGGGATATTCACCATTATCGTTTCAGACCCAC
CTCCCAACCCCGAGGGGACCCGACAGGCCCGAAGGAATAGAAGAAGAAGG
TGGAGAGAGAGACAGAGACAGATCCATTCGATTAGTGAACGGATCTCGAC
GGTATCGATAAGCTAATTCACAAATGGCAGTATTCATCCACAATTTTAAA
AGAAAAGGGGGGATTGGGGGGTACAGTGCAGGGGAAAGAATAGTAGACAT
AATAGCAACAGACATACAAACTAAAGAATTACAAAAACAAATTACAAAAA
TTCAAAATTTTCGGGTTTATTACAGGGACAGCAGAGATCCAGTTTGGGAA
TTAGCTTGATCGATTAGTCCAATTTGTTAAAGACAGGATATCAGTGGTCC
AGGCTCTAGTTTTGACTCAACAATATCACCAGCTGAAGCCTATAGAGTAC
GAGCCATAGATAGAATAAAAGATTTTATTTAGTCTCCAGAAAAAGGGGGG
AATGAAAGACCCCACCTGTAGGTTTGGCAAGCTAGGATCAAGGTTAGGAA
CAGAGAGACAGCAGAATATGGGCCAAACAGGATATCTGTGGTAAGCAGTT
CCTGCCCCGGCTCAGGGCCAAGAACAGTTGGAACAGCAGAATATGGGCCA
AACAGGATATCTGTGGTAAGCAGTTCCTGCCCCGGCTCAGGGCCAAGAAC
AGATGGTCCCCAGATGCGGTCCCGCCCTCAGCAGTTTCTAGAGAACCATC
AGATGTTTCCAGGGTGCCCCAAGGACCTGAAATGACCCTGTGCCTTATTT
GAACTAACCAATCAGTTCGCTTCTCGCTTCTGTTCGCGCGCTTCTGCTCC
CCGAGCTCAATAAAAGAGCCCACAACCCCTCACTCGGCGCGATCTAGATC
TCGAATCGAATTCGAGCTCGGTACCTTTAAGACCAATGACTTACAAGGCA
GCTGTAGATCTTAGCCACTTTTTAAAAGAAAAGGGGGGACTGGAAGGGCT
AATTCACTCCCAACGAAGACAAGATCTGCTTTTTGCTTGTACTGGGTCTC
TCTGGTTAGACCAGATCTGAGCCTGGGAGCTCTCTGGCTAACTAGGGAAC
CCACTGCTTAAGCCTCAATAAAGCTTGCCTTGAGTGCTTCAAGTAGTGTG
TGCCCGTCTGTTGTGTGACTCTGGTAACTAGAGATCCCTCAGACCCTTTT
AGTCAGTGTGGAAAATCTCTAGCAGTAGTAGTTCATGTCATCTTATTATT
CAGTATTTATAACTTGCAAAGAAATGAATATCAGAGAGTGAGAGGAACTT
GTTTATTGCAGCTTATAATGGTTACAAATAAAGCAATAGCATCACAAATT
TCACAAATAAAGCATTTTTTTCACTGCATTCTAGTTGTGGTTTGTCCAAA
CTCATCAATGTATCTTATCATGTCTGGCTCTAGCTATCCCGCCCCTAACT
CCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCT
CTGAGCTATTCCAGAAGTAGTGAGGAGGCTTTTTTGGAGGCCTAGGCTTT
TGCGTCGAGACGTACCCAATTCGCCCTATAGTGAGTCGTATTACGCGCGC
TCACTGGCCGTCGTTTTACAACGTCGTGACTGGGAAAACCCTGGCGTTAC
CCAACTTAATCGCCTTGCAGCACATCCCCCTTTCGCCAGCTGGCGTAATA
GCGAAGAGGCCCGCACCGATCGCCCTTCCCAACAGTTGCGCAGCCTGAAT
GGCGAATGGCGCGACGCGCCCTGTAGCGGCGCATTAAGCGCGGCGGGTGT
GGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCC
CGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTG
GGCCATCGCCCTGATAGACGGTTTTTCGCCCTTTGACGTTGGAGTCCACG
TTCTTTAATAGTGGACTCTTGTTCCAAACTGGAACAACACTCAACCCTAT
CTCGGTCTATTCTTTTGATTTATAAGGGATTTTGCCGATTTCGGCCTATT
GGTTAAAAAATGAGCTGATTTAACAAAAATTTAACGCGAATTTTAACAAA
ATATTAACGTTTACAATTTCC.

### Cell Preparation

Human leukocytes were obtained from apheresis products of consented human subjects pursuant to an Institutional Review Board (IRB) approved process (Cell Processing Core, Cincinnati Children's Hospital, Cincinnati, Ohio). Leukocytes were enriched for CD4+ and CD8+cells using a CD4/8 microbeads and magnetic retention in accordance with manufacturers instructions (Miltenyi Biotec Inc, Auburn, Calif.). Enrichment of cells recovered from the column was determined by flow cytometry using antibodies against CD4 and CD8. Cells recovered from the column were cultured in TEXMACS supplemented with 1% (v/v) of 100× penicillin and streptomycin stock solution (Lonza) and activated and expanded using cross-linking of the T-cell receptor complex (CYTOSTIM, Miltentyi Biotech) and interleukin 2 (IL-2) supplementation in accordance with manufacturer's instructions (Miltenyi Biotech). Following 3 days of stimulation, flow cytometry was performed on the expanded human leukocytes using fluorochrome conjugated antibodies against CD3, CD4 and CD8. Cells were gated on lymphocyte size by selecting forward and side scatter parameters and live cells gated by 7-AAD exclusion. This gated population contained 98% CD3+ cells, 69% CD4+ cells and 28.7% CD8+ cells, indicating the predominant populations were CD3+CD4+ and CD3+CD8+ T-lymphocytes.

Human lymphoid cell line used was RS4;11 (AddexBio, San Diego, Calif.). Cells were adapted to and cultured in TEXMACS Medium (Miltenyi Biotec Inc, Auburn, Calif.) supplemented with 1% (v/v) of 100× penicillin/streptomycin stock solution (Lonza Inc, Allendale, N.J.) and RS4;11 was optionally supplemented with 10% (v/v) heat-inactivated fetal calf serum (HI-FCS) (VWR, Radnor, Pa.).

Cell cultures were incubated at 37° C. in an atmosphere of 5% CO₂ in air.

Stock solutions of steroids and posaconazole were made in high purity dimethyl sulfoxide (DMSO) and stored under argon gas in glass containers at −20° C. until use. Control arms contained a vehicle only control (e.g. DMSO).

### Transfection

Given the frequency with which HSD activity can be conferred on (non-hematopoietic) cells of hamsters (Chinese Hamster Ovary (“CHO”) cells), mice (MLO-Y4 and MC3T3-E1 cells), humans (HEK-293s) and Pichia Pastoris, it was unexpected when no detectable resistance to steroid was conferred by transfecting human lymphoid cells with expression vectors encoding human HSD11B1, HSD11B2, ten HSD11B1L variants as well as the HSD11B1L reference sequence for chimpanzee (Pan troglodytes) (denoted herein as HSD11B1Lchimp; SEQ ID NO: 14) and the modified transcortin SER6mod.

Cells were electroporated on a 4D-NUCLEOFECTOR X system (Lonza Walkersville Inc, Walkersville Md.) according to manufacturer's instructions. Between 1E05 to 7.5E04 cells in 20 μl volume of electroporation solution and 0.5 to 0.75 pg of vector DNA or 0.5 pg of manufacturer's green fluorescent protein (GFP) control vector were added to each well. The 16 well NUCLEOCUVETTE strip was used with electroporation solution SF Cell Line Media and electroporation setting DC-100. Following electroporation, cells were transferred to warmed culture media and incubated overnight. Assessment of transfection efficiency and cell viability was performed within 24 hours of electroporation by measuring green fluorescence protein and 7AAD dye exclusion using flow cytometry on cells transfected with the GFP control vector.

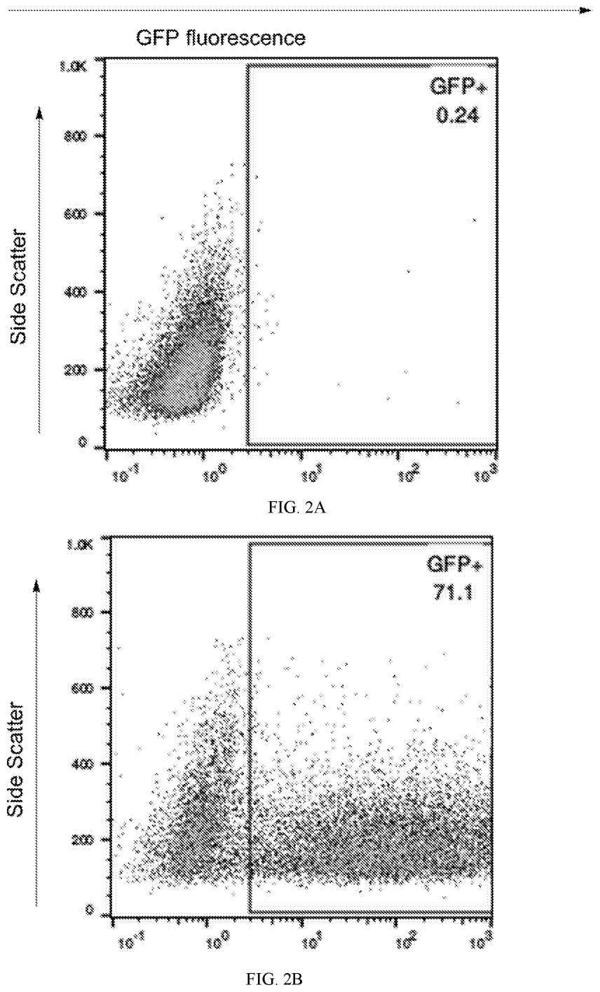

FIGS. 2A and 2B depict the transduction efficiency. Flow cytometry of lymphoid cells electroporated with vector containing GFP performed in parallel to electroporation with vectors containing cloned HSD genes. Green fluorescent protein detection in cells electroporated with a GFP containing vector (right panel) compared to electroporation with non-GFP vector (left panel) demonstrates the electroporation conditions supported efficient gene transfer.

### Cell Survival/Killing Assay

One thousand cells per well were placed in 96 U-bottom plates and drugs added. After incubation for 48 hours, cell number was determined using luminescent assays that measure the intracellular ATP in intact cells (CELLTITER-GLO or CELLTITER-GLO 2.0) (Promega, Madison, Wis.). In accordance with manufacturer's instructions, cell lysates were transferred to white multiwell plates and read on a PerkinElmer ENVISION 2103 Multilabel plate reader. Data was captured via Perkin Elmer EnVision software, and expressed as a proportion of their respective controls where the control value was 100 percent.

Table 2 shows the survival (percentage) of cells transfected with genes that were further challenged with steroids. RS4;11 cells were electroporated with vectors for the genes: human HSD11B1L variants ‘a’ through ‘i’ (SEQ ID NOs: 13, 9, 12, 10, 7, 11, 8, 6, and 5, respectively), chimpanzee HSD11B1L (chimp) (SEQ ID NO: 14 for protein; SEQ ID NO: 19 for DNA), human HSD11B1 (HSD11B1; SEQ ID NO: 4) and HSD11B2 (HSD11B2; SEQ ID NO:3) genes and the modified transcortin SER6mod (SEQ ID NO: 1). Cells were incubated with 100 nM of dexamethazone (DEX) or prednisolone (PRED) for 48 hours. Cell survival was calculated with respect to gene electroporated cells that were not treated with steroids (treated with vehicle alone) (control, deemed 100%). Values are expressed as mean percentage of control and standard deviation (+/−). This shows electroporated genes did not result in “protection”, or resistance to DEX or PRED. It was surprising that using electroporation (typically a transient expression) HSD11B2 did not confer detectable protection given the HSD11B2 transfection studies in other cell types as shown by others to generate enzyme activity. However, the use of lenti-transduction (the same vector packaged into a lenti particle, followed by integration into the target cell genome) was needed to show a result.

TABLE 2
Survival (percentage) of cells transfected with genes and challenged
with dexamethasone (DEX) or prednisolone (PRED), compared to
transfected cells treated with vehicle alone without steroids (deemed
100%). Values expressed as mean percentage of control ±
standard deviation.

| Transfected with Gene that encodes protein | | |
|---|---|---|
| sequence: | DEX | PRED |
| HSD11B1L isoform a (SEQ ID NO: 13) | 4.5 ± 0.49 | 22.5 ± 5.6 |
| HSD11B1L isoform b (SEQ ID NO: 9) | 3.7 ± 0.17 | 22.5 ± 2.2 |
| HSD11B1L isoform c (SEQ ID NO: 12) | 4.8 ± 0.53 | 26.8 ± 3.9 |
| HSD11B1L isoform d (SEQ ID NO: 10) | 4.4 ± 0.29 | 24.6 ± 2.7 |
| HSD11B1L isoform e (SEQ ID NO: 7) | 5.0 ± 0.64 | 27.5 ± 3.7 |
| HSD11B1L isoform f (SEQ ID NO: 11) | 7.1 ± 0.64 | 40.1 ± 5.5 |
| HSD11B1L isoform g (SEQ ID NO: 8) | 5.6 ± 0.39 | 26.2 ± 3.5 |
| HSD11B1L isoform h (SEQ ID NO: 6) | 4.5 ± 0.75 | 26.0 ± 2.4 |
| HSD11B1L isoform i (SEQ ID NO: 5) | 4.6 ± 0.33 | 27.4 ± 1.8 |
| HSD11B1Lchimp GBLOCK ® (SEQ ID NO: 19 | 5.8 ± 0.92 | 31.6 ± 4.9 |
| for gene) | | |
| HSD11B1Lchimp (SEQ ID NO: 14 for protein) | | |
| Corticosteroid 11-beta-dehydrogenase isozyme 1 | 4.6 ± 0.53 | 24.0 ± 4.6 |
| (human HSD11B1; SEQ ID NO: 4) | | |
| Corticosteroid 11-beta-dehydrogenase isozyme 2 | 4.4 ± 0.54 | 21.9 ± 9.7 |
| (human HSD 2; SEQ ID NO: 3) | | |
| SER6mod (SEQ ID NO: 1) | 5.1 ± 0.65 | 28.1 ± 7.1 |

### Production of Recombinant Lentivirus

HSD11B1, HSD11B2 and apool of HSD11B1L vectors were used to generate lentiviral particles. Vectors containing the GBLOCKS® encoding HSD11B1, HSD11B2 and a pool of eleven other vectors (with GBLOCKS® encoding HSD1B1L variants a, b, c, d, e, f, g, h, i, chimp and SER6mod) were added to a system used to generate lentiviral particles pseudotyped with Vesicular stomatitis Indiana virus G protein (VSV-G) (Cincinnati Children's Hospital Viral Vector Core, Cincinnati, Ohio). Three separate supernatants containing viral particles with HSD11B1 (“BONE”), HSD11B2 (“BTWO”) and the pool of eleven vectors (“THREE-MIX”) were concentrated by ultracentifugation. Titer was measured using ap24 dipstick (Lenti-X GoStix, Takara Bio USA, Mountain View, Calif.).

Target cells (lymphoid RS4;11) were exposed to viral particles at multiplicity of infection (MOI) of 3 for two hours and then diluted with 6× volume of cell culture medium, incubated overnight, then washed by centrifugation and resuspended in conditioned cell culture media for continued culture prior to use in assays.

### Testing Lenti-Transduced Cells

Depending on the experiment, between 500 to 1,000 cells per well were placed in 96 U-bottom plates and drugs added. After incubation for 48 hours, cell number was determined using luminescent assays that measure the intracellular ATP in intact cells (CELLTITER-GLO or CELLTITER-GLO 2.0) (Promega, Madison, Wis.). In accordance with manufacturer's instructions, cell lysates were transferred to white multiwell plates and read on a PerkinElmer ENVISION 2103 Multilabel plate reader. Data was captured via Perkin Elmer EnVision software and expressed as a proportion of their respective controls where the control value was 100 percent.

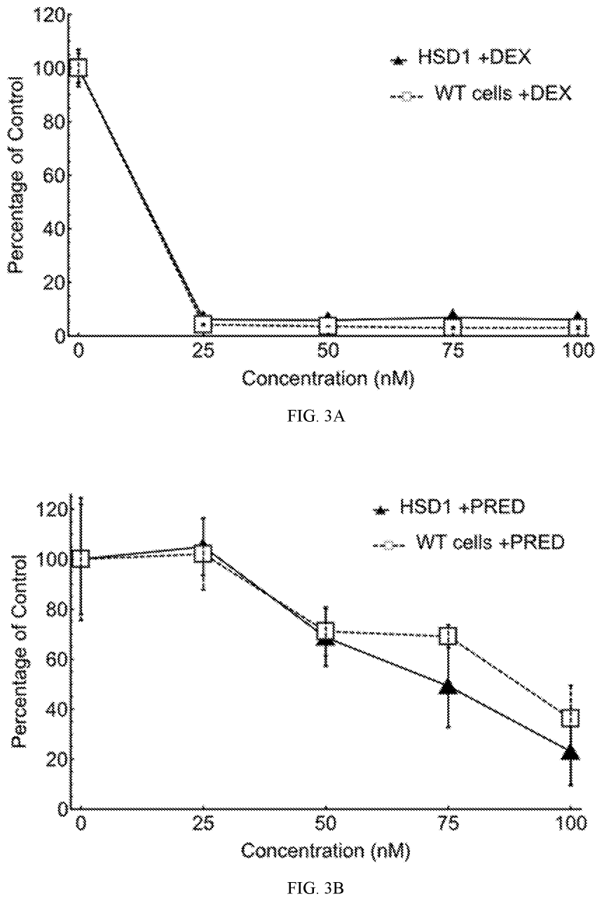

Following transduction with HSD11B1 lenti “BONE”, the genetically modified lymphoid cells did not exhibit resistance to dexamethasone (DEX) or prednisolone (PRED), as the survival of these RS4;11 cells transduced with the HSD11B1 lenti-vector in the presence of DEX or PRED, at various concentrations, were similar to that of the wild-type RS4;11 cells (FIGS. 3A and 3B).

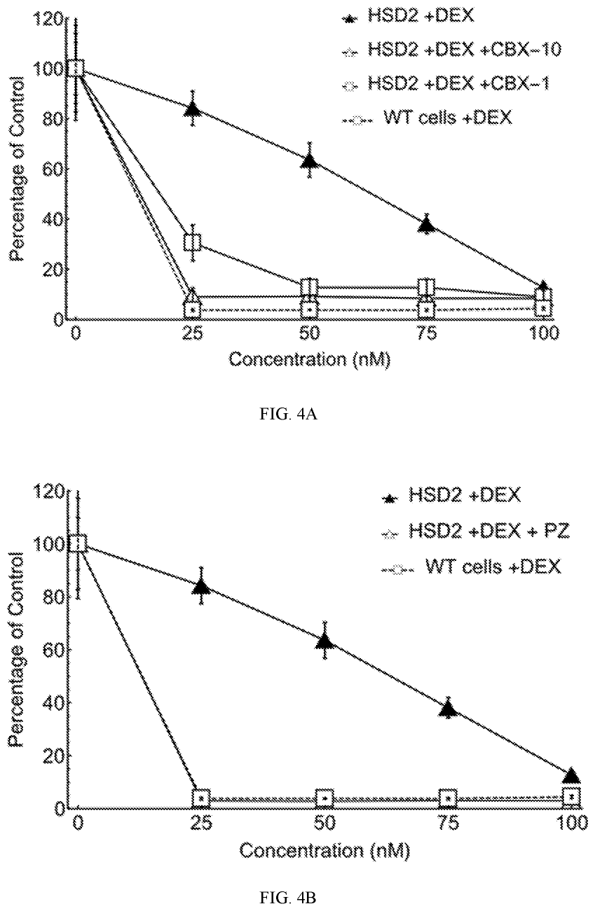

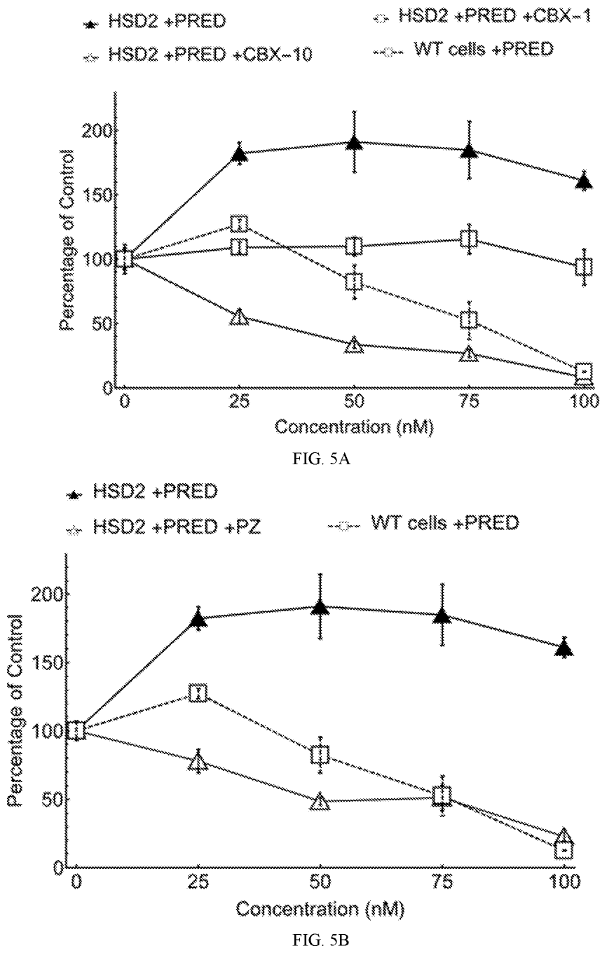

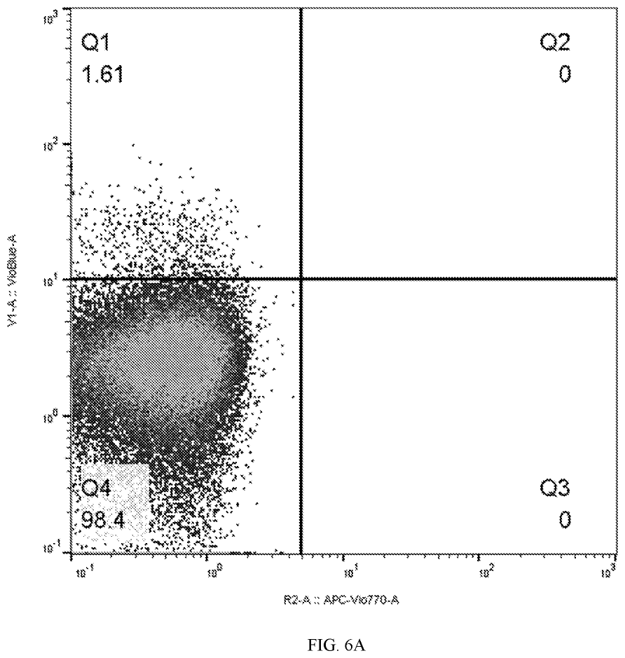

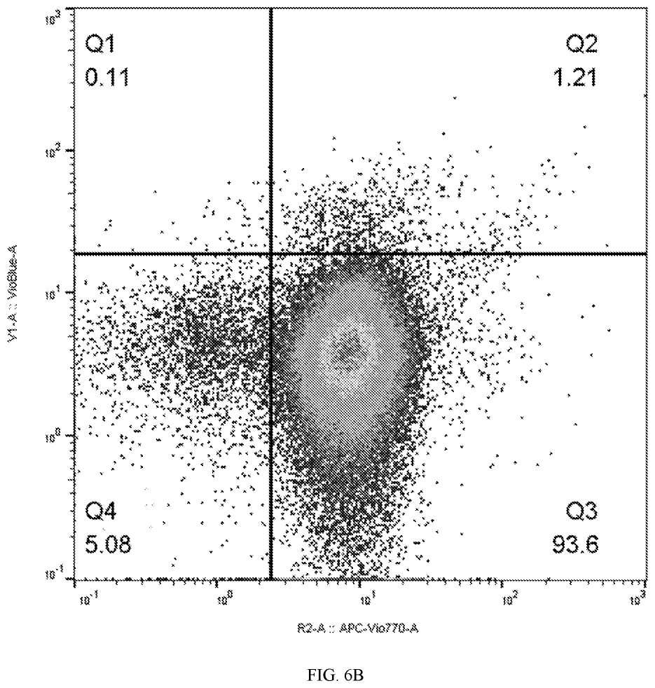

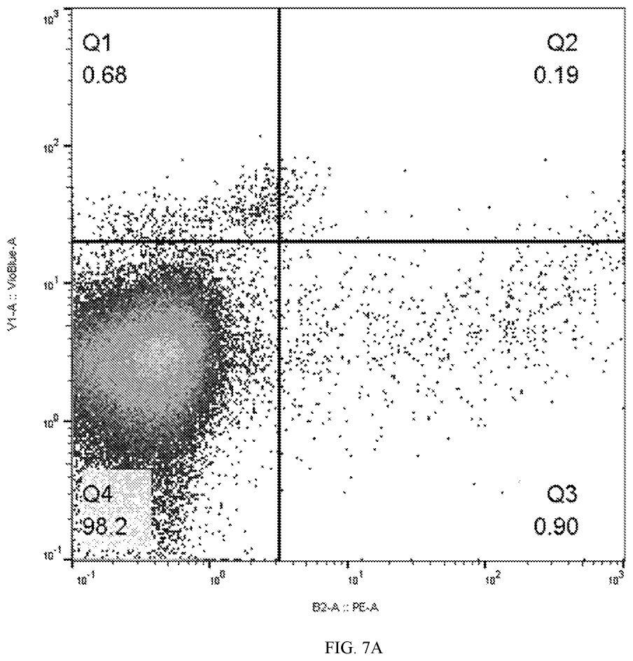

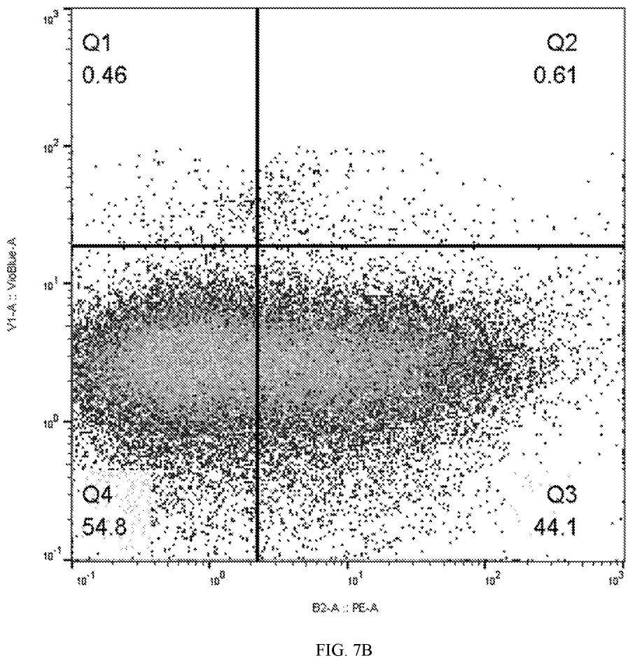

Following transduction with HSD11B2 lenti “BTWO”, the genetically modified lymphoid cells demonstrated protection (resistance) against steroids. FIGS. 4A and 4B show HSD11B2-transduced cells exhibit near complete protection against (resistance to) the effects of dexamethasone compared to control arms. FIGS. 5A and 5B show HSD11B2-transduced cells exhibit near complete protection against (resistance to) the effects of prednisolone compared to control arms. Steroid protection was reduced or eliminated by dosing transduced cells with exemplary HSD inhibitors, posaconazole (PZ; FIGS. 4B and 5B) and carbenoxolone (CBX; FIGS. 4A and 5A). Carbenoxolone was tested at two doses (1 μM denoted as “CBX-1”, and 10 μM denoted as “CBX-10”) and showed a dose response effect. The protection demonstrated here was substantially greater than the marginal result described in Zhang H et al., with adherent mouse cells in Biochemical and Biophysical Research Communications 490 (2017) 1399-1406.

### Additional Assays

Cells transduced with the HSD11B1Lpool lenti would be subject to selection in steroids. Any populations that grow out would be tested for resistance and the gene which conferred resistance would be identified. This was contemplated to flush out an HSD11B1L gene.

The influence of HSD11B2 or HSD11B1 on gene expression was contemplated. Human leukocyte specimens from donor 1 (male, for example) would be split—one half transduced with HSD11B1, other half mock treated. Donor 2 (female) same, split, one half transduced with HSD11B2 and the other half mock treated. All 4 populations would be pooled and contacted with steroid, for a period of hours, followed by single-cell based RNA-sequencing as exemplified by the 10× system (10× GENOMICS, Pleasanton, Calif.). Since we could differentiate each individual (e.g., via analysis of single cell gene expression of sex-specific genes or human leukocyte antigen (HLA) haplotypes and by computationally clustering individual cells into cell types by their global gene expression profile) and whether the cell expresses an HSD, this would show, within individuals, within their identifiable cell subpopulations, and between HSD11B1 (no expected effect on steroid response, i.e., the control arm) and HSD11B2 (showing protection against steroid was conferred) the effects on steroid-responsive gene expression. This could be performed in vitro or in immuno-compromised mice.

Single cell gene expression (10×), RNA-Seq analysis or similar formats would be obtained from core laboratories at commercial vendors or academic core laboratories. The conferring of glucocorticoid resistance is assessed by methods including:

(i) survival in the presence of glucocorticoids, measured by methods including but not limited to: cell counts, cell viability, MTT assay, apoptosis assays (tube or plate-based, imaging, flow cytometry);
(ii) using techniques to measure reduction in immune activity;
(iii) cells lines that die in the presence of glucocorticoids;
(iv) monitoring the translocation of GR from the cytoplasm to the nucleus;
(v) reporter genes and constructs that ‘report’ glucocorticoid action via GR. Campana et al related materials available from DiscoveRx and its PathHunter® CHO-K1 GR Nuclear Translocation Cell Line used in accordance with manufacturer's instructions, Affymetrix (Thermo-Fischer) or other vendors);
(iv) analysis of gene expression by measuring the altered expression of genes that are responsive to signals generated by the GR;
(v) measurement of the decreased levels of 11-hydroxy steroids and (vi) measurement of increased levels of 11-keto steroids.

### Clinical Application

In general, if adoptively transferred gene modified leukocytes are resistant to glucocorticoids while cells in the recipient remained responsive to the immunosuppressive effects of glucocorticoids, the administration of glucocorticoids to such a patient would reduce the function of such endogenous cells while preserving the activity of the adoptively transferred gene modified resistant leukocytes.

For example, in allogeneic kidney transplantation, patients may be medicated with immunosuppressive drugs such as cyclosporin or tacrolimus in combination with a glucocorticoid to prevent rejection of the graft by components of the immune system. Immunocompromised patients are at risk of developing uncontrolled viral infections such as BK virus (BKV) and human cytomegalovirus (CMV). For example, recipients of bone marrow (BMT) or renal transplants, may experience severe renal and urological complications from BKV. There is currently no effective therapeutic for BKV, and the only treatment available in solid organ transplantation is a reduction of immunosuppressive treatment—including glucocorticoids—with the concomitant risk of graft rejection.

Anti-viral T cells can be generated from peripheral blood obtained from a bone marrow donor against CMV, EBV or adenovirus. To make these anti-viral T cells, bone marrow donor blood leukocytes are exposed to viral antigens in vitro using recombinant adenoviral vectors modified to express CMV proteins, and EBV lymphoblastoid cell lines. During this process, the cell population can be transduced or transected with vectors containing a gene for HSD11B2, HSD11B1 or one of the genes for HSD11B1L isoforms, or SER6mod.

Glucocorticoid resistant anti-viral T cells can be infused into a patient. A preferred embodiment is where at least ten percent of genetically modified leukocytes express a gene that confers resistance to 11-beta-hydroxysteroids.

In glioblastoma, glucocorticoids are used to reduce cerebral edema. Gene modified leukocytes (e.g. CAR-T cells) may be used to treat the tumor. If adoptively transferred gene modified anti-tumor leukocytes are also resistant to glucocorticoids while cells in the recipient remained responsive to the immunosuppressive effects of glucocorticoids, the administration of glucocorticoids to such a patient would reduce the function of such endogenous cells while preserving the activity of the adoptively transferred gene modified glucocorticoid resistant anti-tumor leukocytes. To make these anti-glioblastoma leukocytes, during their ex vivo culture the cell population can be transduced or transected or co-transduced or co-transfected with vectors containing a gene for HSD11B2 or one of the gene for HSD11B1L isoforms, or SER6mod. A preferred embodiment is where at least ten percent of genetically modified leukocytes express a gene that confers resistance to 11-beta-hydroxysteroids.

In anti-tumor immunotherapy, certain tumors may contain cells that suppress or regulate anti-tumor cells, thereby reducing the effectiveness of an anti-tumor response.

In general, if adoptively transferred leukocytes had resistance to glucocorticoids but suppressor or regulatory cells within a tumor remained responsive to the immunosuppressive effects of glucocorticoids, the administration of glucocorticoids to a patient would reduce the function of suppressor/regulatory cells while preserving the effector activity of the adoptively transferred gene modified leukocytes. To make these anti-tumor leukocytes, during their ex vivo culture the cell population can be transduced or transected or co-transduced or co-transfected with vectors containing a gene for HSD11B2 or one of the gene for HSD11B1L isoforms, or SER6mod. A preferred embodiment is where at least ten percent of genetically modified leukocytes express a gene that confers resistance to 11-beta-hydroxysteroids.

In adoptive immunotherapy, transferred cells may produce undesirable side effects and treatment of these side effects may include the use of glucocorticoids including dexamethasone. Other approaches to controlling undesired adoptive cell function have used cell surface markers or suicide genes.

In any of the forgoing examples of clinical use, and in the application of this invention, resistance to glucocorticoids of genetically modified leukocytes expressing HSD11B2, an HSD11B1L variant or HSD11B1 can be reduced by administration to the patient of a preferred inhibitor of the enzyme.

In any of the forgoing examples of clinical use, and in the application of this invention, resistance to glucocorticoids of genetically modified leukocytes expressing SER6mod can be overcome by administration to the patient of increased levels of glucocorticoids, including dexamethasone.

Sequences of SEQ ID Nos: 1-14 are shown below. (Sequences of SEQ ID Nos: 15-30 are shown above.)

TABLE 3
Sequences of SEQ ID Nos: 1-14.

| SEQ ID NO. | Name | Sequences |
|---|---|---|
| 1 | Protein encoded | MDPNAAYVNMSNHHRGLASANVDFAFSLYK |
| | by SER6mod | HLVALSPKKNIFISPVSISMALAMLSLGTC |
| | gene. | GHTRAQLLQGLGFNLTERSETEIHQGFQHL |
| | (begins at residue | HQLFAKSDTSLEMTMGNALFLDGSLELLES |
| | 23 of transcortin) | FSADIKHYYESEVLAMNFQDWATASRQINS |
| | | YVKNKTQGKIVDLFSGLDSPAILVLVNYIF |
| | | FKGTWTQPFDLASTREENFYVDETTVVKVP |
| | | MMLQSSTISYLHDAELPCQLVQMNYVGNGT |
| | | VFFILPDKGKMNTVIAALSRDTINRWSAGL |
| | | TSSQVDLYIPKVTISGVYDLGDVLEEMGIA |
| | | DLFTNQANFSRITQDAQLKSSKVVHKAVLQ |
| | | LNEEGVDTAGSTGVTLNLTSKPIILRFNQP |
| | | FIIMIFDHFTWSSLFLARVMNPV |
| 2 | Transcortin | MPLLLYTCLLWLPTSGLWTVQAMDPNAAYV |
| | | NMSNHHRGLASANVDFAFSLYKHLVALSPK |
| | | KNIFISPVSISMALAMLSLGTCGHTRAQLL |
| | | QGLGFNLTERSETEIHQGFQHLHQLFAKSD |
| | | TSLEMTMGNALFLDGSLELLESFSADIKHY |
| | | YESEVLAMNFQDWATASRQINSYVKNKTQG |
| | | KIVDLFSGLDSPAILVLVNYIFFKGTWTQP |
| | | FDLASTREENFYVDETTVVKVPMMLQSSTI |
| | | SYLHDAELPCQLVQMNYVGNGTVFFILPDK |
| | | GKMNTVIAALSRDTINRWSAGLTSSQVDLY |
| | | IPKVTISGVYDLGDVLEEMGIADLFTNQAN |
| | | FSRITQDAQLKSSKVVHKAVLQLNEEGVDT |
| | | AGSTGVTLNLTSKPIILRFNQPFIIMIFDH |
| | | FTWSSLFLARVMNPV |
| 3 | Corticosteroid | MERWPWPSGGAWLLVAARALLQLLRSDLRL |
| | 11-beta- | GRPLLAALALLAALDWLCQRLLPPPAALAV |
| | dehydrogenase | LAAAGWIALSRLARPQRLPVATRAVLITGC |
| | isozyme 2 | DSGFGKETAKKLDSMGFTVLATVLELNSPG |
| | (HSD 1 1B2) | AIELRTCCSPRLRLLQMDLTKPGDISRVLE |
| | | FTKAHTTSTGLWGLVNNAGHNEVVADAELS |
| | | PVATFRSCMEVNFFGALELTKGLLPLLRSS |
| | | RGRIVTVGSPAGDMPYPCLGAYGTSKAAVA |
| | | LLMDTFSCELLPWGVKVSIIQPGCFKTESV |
| | | RNVGQWEKRKQLLLANLPQELLQAYGKDYI |
| | | EHLHGQFLHSLRLAMSDLTPVVDAITDALL |
| | | AARPRRRYYPGQGLGLMYFIHYYLPEGLRR |
| | | RFLQAFFISHCLPRALQPGQPGTTPPQDAA |
| | | QDPNLSPGPSPAVAR |
| 4 | Corticosteroid | MAFMKKYLLPILGLFMAYYYYSANEEFRPE |
| | 11-beta- | MLQGKKVIVTGASKGIGREMAYHLAKMGAH |
| | dehydrogenase | VVVTARSKETLQKVVSHCLELGAASAHYIA |
| | isozyme 1 | GTMEDMTFAEQFVAQAGKLMGGLDMLILNH |
| | (HSD11B1) | ITNTSLNLFHDDIHHVRKSMEVNFLSYVVL |
| | | TVAALPMLKQSNGSIVVVSSLAGKVAYPMV |
| | | AAYSASKFALDGFFSSIRKEYSVSRVNVSI |
| | | TLCVLGLIDTETAMKAVSGIVHMQAAPKEE |
| | | CALEIIKGGALRQEEVYYDSSLWTTLLIRN |
| | | PCRKILEFLYSTSYNMDRFINK |
| 5 | hydroxysteroid | MKVLLLTGLGALFFAYYWDDNFDPGGLDYL |
| | 11-beta- | VLNHIGGAPAGTRARSPQATRWLMQVNFVS |
| | dehydrogenase | YVQLTSRALPSLTDSKGSLVVVSSLLGRVP |
| | 1-like protein | TSFSTPYSAAKFALDGFFGSLRRELDVQDV |
| | isoform i | NVAITMCVLGLRDRASAAEAVRSSTSRPRQ |
| | precursor | PEHRGVPLQSQTAMFLPPTVPGARTLTETP |
| | (HSD11B1Li) | LRGWPQPKMKSSRQKSKTEKNDGHLEPVTA |
| | | WEVQVPRVRRLCRGLARPHLFGHD |
| 6 | hydroxysteroid | MKVLLLTGLGALFFAYYWDDNFDPGKLCEL |
| | 11-beta- | RATDVAGAAQPDGQQGLPGGGVLAARPRAH |
| | dehydrogenase | VVLHSLLGGQVCAGRLLRLPAAGAGRAGRE |
| | 1-like protein | RGHHHVRPGPPRSRLRRRGSQGSHEGQGGP |
| | isoform h | GAQGSPGRDPRRRHARGRRLLPVAFPPAVL |
| | precursor | APALATAPAGLVYPPGAQRHGRGSLSTGGC |
| | (HSD11B1Lh) | PSSPRRQCSSLQLSLEPEHSQRHP |
| 7 | hydroxysteroid | MKVLLLTGLGALFFAYYWDDNFDPASLQGA |
| | 11-beta- | RVLLTGANAGVGEELAYHYARLGSHLVLTA |
| | dehydrogenase | HTEALLQKVVGNCRKLGAPKVFYIAADMAS |
| | 1-like protein | PEAPESVVQFALDKLGGLDYLVLNHIGGAP |
| | isoform e | AGTRARSPQATRWLMQVNFVSYVQLTSRAL |
| | (HSD11B1Le) | PSLTDSKGSLVVVSSLLGRVPTSFSTPYSA |
| | | AKFALDGFFGSLRRELDVQDVNVAITMCVL |
| | | GLRDRASAAEAVRSSTSRPRQPEHRGVPLQ |
| | | SQTAMFLPPTVPGARTLTETPLRGWPQPKM |
| | | KSSRQKSKTEKNDGHLEPVTAWEVQVPRVR |
| | | RLCRGLARPHLFGHD |
| 8 | hydroxysteroid | MANLGTLQLLPPRFKRFSCLSLPNIWITGM |
| | 11-beta- | PVPATSVPCPSAGPHRTMKVLLLTGLGALF |
| | dehydrogenase | FAYYWDDNFDPASLQGARVLLTGANAGVGE |
| | 1-like protein | ELAYHYARLGSHLVLTAHTEALLQKVVGNC |
| | isoform g | RKLGAPKVFYIAADMASPEAPESVVQFALD |
| | (HSD11B1Lg) | KLGGLDYLVLNHIGGAPAGTRARSPQATRW |
| | | LMQVNFVSYVQLTSRALPSLTDSKGSLVVV |
| | | SSLLGRVPTSFSTPYSAAKFALDGFFGSLR |
| | | RELDVQDVNVAITMCVLGLRDRASAAEAVR |
| | | GVTRVKAAPGPKAALAVIRGGATRAAGVFY |
| | | PWRFRLLCLLRRWLPRPRAWFIRQELNVTA |
| | | AAA |
| 9 | hydroxysteroid | MKVLLLTGLGALFFAYYWDDNFDPASLQGA |
| | 11-beta- | RVLLTGANAGVGEELAYHYARLGSHLVLTA |
| | dehydrogenase | HTEALLQKVVGNCRKLGAPKVFYIAADMAS |
| | 1-like protein | PEAPESVVQFALDKLGGLDYLVLNHIGGAP |
| | isoform b | AGTRARSPQATRWLMQVNFVSYVQLTSRAL |
| | precursor | PSLTDSKGSLVVVSSLLGRVPTSFSTPYSA |
| | (HSD1B1Lb) | AKFALDGFFGSLRRELDVQDVNVAITMCVL |
| | | GLRDRASAAEAVRGVTRVKAAPGPKAALAV |
| | | IRGGATRAAGVFYPWRFRLLCLLRRWLPRP |
| | | RAWFIRQELNVTAAAA |
| 10 | hydroxysteroid | MQVNFVSYVQLTSRALPSLTDSKGSLVVVS |
| | 11-beta- | SLLGRVPTSFSTPYSAAKFALDGFFGSLRR |
| | dehydrogenase | ELDVQDVNVAITMCVLGLRDRASAAEAVRG |
| | 1-like protein | VTRVKAAPGPKAALAVIRGGATRAAGVFYP |
| | isoform d | WRFRLLCLLRRWLPRPRAWFIRQELNVTAA |
| | (HSD 1B 1Ld) | AA |
| 11 | hydroxysteroid | MQVNFVSYVQLTSRALPSLTDSKGSLVVVS |
| | 11-beta- | SLLGRVPTSFSTPYSAAKFALDGFFGSLRR |
| | dehydrogenase | ELDVQDVNVAITMCVLGLRDRASAAEAVRS |
| | 1-like protein | STSRPRQPEHRGVPLQSQTAMFLPPTVPGA |
| | isoform f | RTLTETPLRGWPQPKMKSSRQKSKTEKNDG |
| | (HSD11B1Ld) | HLEPVTAWEVQVPRVRRLCRGLARPHLFGH |
| | | D |
| 12 | hydroxysteroid | MASPEAPESVVQFALDKLGGLDYLVLNHIG |
| | 11-beta- | GAPAGTRARSPQATRWLMQVNFVSYVQLTS |
| | dehydrogenase | RALPSLTDSKGSLVVVSSLLGRVPTSFSTP |
| | 1-like protein | YSAAKFALDGFFGSLRRELDVQDVNVAITM |
| | isoform c | CVLGLRDRASAAEAVRGVTRVKAAPGPKAA |
| | (HSD11B1Lc) | LAVIRGGATRAAGVFYPWRFRLLCLLRRWL |
| | | PRPRAWFIRQELNVTAAAA |
| 13 | hydroxysteroid | MKVLLLTGLGALFFAYYWDDNFDPGGLDYL |
| | 11-beta- | VLNHIGGAPAGTRARSPQATRWLMQVNFVS |
| | dehydrogenase | YVQLTSRALPSLTDSKGSLVVVSSLLGRVP |
| | 1-like protein | TSFSTPYSAAKFALDGFFGSLRRELDVQDV |
| | isoform a | NVAITMCVLGLRDRASAAEAVRGVTRVKAA |
| | precursor | PGPKAALAVIRGGATRAAGVFYPWRFRLLC |
| | (HSD11B1La) | LLRRWLPRPRAWFIRQELNVTAAAA |
| 14 | HSD11B1L | MKVLLLTGLGALFFAYYWDDNFDPASLQGA |
| | chimp | RVLLTGANAGVGEELAYHYARLGSHLVLTA |
| | | HTEALLQKVVGNCRKLGAPKVFYIAADMAS |
| | | PEAPESVVQFALDKLGEGLGLNPGVRDRGL |
| | | GLRDRTRIGLWCRLQVNFVSYVQLTSRALP |
| | | SLTDSKGSLVVVSSLLGRVPTSFSTPYSAA |
| | | KFALDGFFGSLRRELDVQDVNVAITMCVLG |
| | | LRDRASAAEAVRGVTRVKAAPGPKAALAVI |
| | | RGGATRAAGVFYPWRFRLLCLLRRWLPRPR |
| | | AWFIRQELNVTAAAAA |

### Example 2

### Construct/Vector Design

Synthetic DNA oligomers were obtained from GeneWiz (South Plainfield, N.J.) and IDT. Using standard cloning techniques and sequence verification, synthetic DNA sequences were used to construct: (i) a modified HSD11B2 gene wherein the sequence corresponding to approximately exon 1 of HSD111B2 was not codon modified but remained “wild type”, such modified sequence was named “wtExon1-none” (asset forth in SEQ ID No.: 31), and (ii) a bicistronic sequence encoding HSD11B2 plus a sequence encoding a cell surface marker (“tag”) with an intervening “2A” sequence encoding a “self-cleaving peptide”, such bicistronic sequence was named “B2-Tag” (polynucleotide set forth in SEQ ID No.: 32, which encodes a polypeptide set forth in SEQ ID No.: 33). Separate polypeptides can be obtained from the translation of a single RNA which where one or more 2A sequences are placed between regions encoding the distinct polypeptides or proteins. During ribosomal translation, the growing polypeptide can undergo cleavage in region of the 2A peptide. Specifically, the intervening self-cleaving coding sequence herein comprised two 2A sequences in tandem (T2A-P2A) located between a pair of genes, that is, a “bicistronic” construct of the general structure: first coding region—tandem 2A region—second coding region. Single 2A sequences such as T2A, P2A or E2A, doublets (e.g., P2A-T2A), triplets (e.g., P2A-T2A-E2A) or more groups of 2A sequences can also be used in the 2A region instead of a tandem region. Multiple separate peptides can also be expressed using 2A regions between the desired coding regions. Liu et al. showed examples of 3 or 4 separate proteins being expressed through the use of 2 or 3 intervening 2A regions, respectively, interspersed in a single transcript. In other words, tri- and quad-cistronic constructs.

Polypeptides generated by the use of 2A regions can be expected to include amino acids derived from the 2A region which remain following self-cleavage of the 2A region or regions. Studies have shown the 2A regions that remain post-cleavage are found at the C-terminus of the peptide or protein encoded in the sequence before (i.e., at the 5′ of) the 2A region and at the N-terminus of the peptide or protein encoded in the sequence following (i.e., at the 3′ of) a 2A region.

Due to common amino sequences in 2A sequences (e.g., C-terminal NPGP using the single letter amino acid code) alternate codon usage may be required to encode 2A regions to avoid interference with the function of a gene construct, for example, by homologous or complementary sequences impeding cloning, preventing proper vector generation and function or generating secondary structure within the RNA transcript or with a delivered nucleic acid such as an RNA that may directly encode a protein.

Incorporation of a cell surface tag into a poly-cistronic gene construct permits the detection of transduction of the gene construct as well as isolation of cells using isolation techniques such as flow cytometry or bead-based purification techniques, where such isolation techniques are generally known in the field. Detection of expressed proteins, including cell surface tags, is afforded through reagents with antigen or epitope-specific affinity such as antibodies or aptamers. The detection of the cell surface tag gives a strong indication that cleavage has occurred and that gene products are present in the cell. Intracellular antigens can be detected following permeabilization of a cell and may be conducted instead of, or in concert with, detection of antigens expressed on the cell surface. Binding of epitope-specific affinity reagent to its target antigen or epitope can be detected by standard methods including direct or indirect means that are well known in the field including flow cytometry, mass cytometry, ELISA, enzyme assays and fluorescent microscopy.

Examples of direct detection include, but are not limited to, creating a modified antibody which contains structures that can be detected such as fluorophore dyes and quantum dots, stable isotopes, nucleic acid tags, nuclear magnetic resonance (NMR) tags, enzymes including ribozymes, biotin, and radioisotope. Indirect means include using reagents that bind to the primary antibody. Examples of indirect detection include, but are not limited to, detecting binding of a primary antibody using a secondary reagent where such secondary reagent is labeled and where such labels can be chosen from the list of detection materials including but not limited to fluorophore dyes and quantum dots, stable isotopes, nucleic acid tags, nuclear magnetic resonance (NMR) tags, enzymes including ribozymes, biotin and radioisotope. Examples of secondary reagents include but is not limited to avidin, antisera, monoclonal antibodies, aptamers, Fc region binding proteins, anti-idiotype antibodies and peptides that bind to grooves, clefts or pockets in antibody structures or aptamers.

Chessie 13-39.1 is a linear epitope that was originally mapped to amino acids 252-273 (RPVVSTQLLLNGSLAEEEVVIR; SEQ ID No.: 34) of gp160 of the LAI strain of human immunodeficiency virus one (HIV-1). Chessie 13-39.1 epitope can be bound with antibodies from the Anti-HIV-1 gp160 Hybridoma (Chessie 13-39.1; IgG1)(NIH AIDS Reagent Program, catalog number 1209, lot number 040144, antibody deposited by Dr. George Lewis). Binding of Chessie 13-39.1 antibody or a Chessie 13-39.1 epitope binding primary reagent can be detected by standard methods including direct or indirect means.

Amino acid sequences from the granulocyte-macrophage colony-stimulating factor (GM-CSF) leader sequence have been used to express cell surface proteins from transgenes (U.S. Pat. No. 8,802,374 to M. C. Jensen, which is incorporated by reference herein in its entirety).

CD8a is a well characterized single chain protein that is primarily expressed on the cell surface of T-lymphocytes. CD8a has an extracellular region, a transmembrane span and an intracellular signaling tail. Forms of CD8a have been described that have a truncated intracellular tail and which lack detectable signaling capability.

One or more of the above-described gene elements in this Example were cloned into linearized pCCL-c-MNDU3c-X lenti-vector backbones using standard cloning techniques and confirmed with Sanger sequencing:

In some embodiments, Applicant's cell surface tag sequence (“tag” or “tag sequence”) has a general structure: polypeptide from GM-CSF leader sequence-spacer sequence-Chessie 13.39.1 epitope-spacer-CD34 derived sequence-small region of CD8a extracellular stalk-spacer-a short intracellular region of CD8a that lacks signaling capacity-stop codon, e.g., an embodiment of the “tag” has a polynucleotide of which as set forth in SEQ ID No.: 35, which translates as a polypeptide sequence as set forth in SEQ ID No.: 36.

In some embodiments, Applicant's sequences of the “HSD11B2-2A regions-tag” insert (“tag” is downstream of HSD11B2) has a general structure of: region homologous to vector-Kozak-ATG-rest of gene encoding HSD11B2 gene without a stop codon-T2AP2A-tag sequence-stop codon-region homologous to vector, e.g., an embodiment has a polynucleotide sequence as set forth in SEQ ID No.: 32, which translates as a polypeptide sequence as set forth in SEQ ID No.:33.

In some embodiments, Applicant's sequences of the combined “tag-2A regions-HSD11B2” insert (“tag” is upstream of HSD11B2) have a general structure of: region homologous to vector-Kozak-ATG-tag sequence gene without a stop codon-T2AP2A-codon optimized HSD11B2 gene-stop codon-region homologous to vector, e.g., an embodiment has a polynucleotide sequence as set forth in SEQ ID No.: 37, which translates to a polypeptide sequence as set forth in SEQ ID No.: 38.

For clarity, Kozak sequences overlap with start ATG (Met) codon and the first base of the codon following the ATG. Furthermore, the ATG start codon is shown here as a separate region solely for clarity and as seen in the sequence listings, it is not indicative of two consecutive ATG start codons.

### Cell Preparation and Transduction

Human peripheral blood leukocytes (HPBL) were prepared by density gradient centrifugation of anti-coagulated human blood (Cincinnati Children's Hospital Cell Processing Core). HPBL were stimulated with CYTOSTIM™, inteleukin 2 (IL-2) in TEXMACS™ media (all reagents, Miltenyi Biotech) according to manufacturer's instructions for three days. These conditions resulted in a population of cells that were predominantly T-lymphocytes. After stimulation, the entire population of stimulated cells (“stimulated leukocytes”) were cryopreserved in cell freezing media which contained dimethyl sulfoxide (DMSO). Prior to experiments, cryopreserved stimulated leukocytes were thawed, washed twice and cultured in TEXMACS™ media supplemented with 10% (v/v) heat inactivated fetal bovine serum (HI-FBS, VWR). After an overnight incubation, typical cell viability post-thaw was greater than 95% as determined by Trypan blue dye exclusion. Post-thaw, cells were cultured overnight prior to transduction with lentivirus. VSV-G pseudotyped lentiviral particles in supernatant were generated by transfection of 293T cells, either by calcium phosphate precipitation or use of PEIPro (Polyplus-transfection SA, New York, N.Y.) with lenti-vector and helper plasmids of the Delta 8.9 system which includes (Viral Vector Core, Cincinnati Children's Hospital). Two rounds of culture supernatant were collected from 293T cultures, pooled and virus concentrated by ultracentrifugation (“concentrated viral supernatant”). Multiple small volume aliquots of concentrated viral supernatant were frozen at −80° C. Target cells were concentrated by centrifugation, resuspended in a small volume and transduced with concentrated viral supernatant at an estimated multiplicity of infection (MOI) of 1.0 for two hours at 37° C. Following transduction, cells were diluted in TEXMACS™ with 10% HI-FBS and 100 U/mL IL-2 and cultured at least overnight. In some experiments, a second round of transduction was conducted 24 hours following the first transduction using the same procedure. Following transduction, cells were cultured in TEXMACS™ with 10% HI-FBS and 100 U/mL IL-2 for 48 to 72 hours before use in experiments.

### Assays

For steroid degradation studies, stimulated leukocytes (5×10⁴ per 96 well) were plated into U-bottom 96 well plates in TEXMACS™ with 100 U/ml IL-2. Steroids and enzyme inhibitors were added to the noted final concentrations alongside DMSO-only vehicle controls. Cultures were incubated with these added compounds overnight for 18 to 24 hours depending on the experiment (steroid amount at 100 nM). Presented values are experiments with the same incubation time. Wells were aspirated and supernatants recovered following centrifugation of cells (450 g, 4.5 minutes). Supernatants were frozen at −20° C. until analysis. Cell pellets were frozen at −80° C. and retained for viral copy number determination.

Viral copy number (VCN) was assessed on cell pellets by real-time polymerase chain reaction (RT-PCR) by extracting the genomic DNA and using primers and probes for the R-U5 region of the lentiviral vector. VCN was determined using a cGMP testing protocol conducted by the Translational Trials Support & Development Laboratory of Cincinnati Children's Hospital. VCN gives a value (ratio) of integrated viral genomes per host cell genome. To permit direct comparison of the intrinsic activity of each enzyme construct—independent of any variability of target cell transduction or number of transduced cells in an experimental arm—VCN values were combined with the number of live cells measured at the end of incubation to adjust data to reflect a uniform level of cells carrying the transgene.

Cortisol and prednisolone levels in supernatants were detected using an enzyme-linked immunosorbent assay (ELISA) kit (Cortisol ELISA kit, Enzo Life Sciences, Farmingdale, N.Y.) in accordance with manufacturer's instructions. Tissue culture supernatants were diluted in assay buffer to fall within the linear, dynamic response range of the ELISA. Readout was on a PerkinElmer ENVISION 2103 Multilabel plate reader using 405 nm with path length adjustment at 570 nm. Levels were calculated using a 7-point standard curve using the cortisol standards provided in the kit and curve fitting using four parameter logistic regression using code written on Mathematica software (version 10.0, Wolfram Research, Champagne, Ill.). Adjustment was made for the kit's 129% response to prednisolone compared to cortisol (100%).

Dexamethasone levels in supernatants were detected using an enzyme-linked immunosorbent assay (ELISA) kit (Dexamethasone, Neogen Corporation, Lexington Ky.) in accordance with manufacturer's instructions. Tissue culture supernatants were diluted in assay buffer to fall within the linear, dynamic response range of the ELISA. Readout was on a PerkinElmer ENVISION 2103 Multilabel plate reader at 635 nm. A 8-point standard curve was generated from serial dilutions of a certified reference Dexamethasone standard (CERILLIANT®, Millipore-Sigma, St. Louis, Mo.) recommended by Neogen for use with their kit. A standard curve was obtained by fitting a four parameter logistic regression curve to the standards using code written on Mathematica software (version 10.0, Wolfram Research, Champagne, Ill.). Experimental levels were calculated from the standard curve.

Cortisol and cortisone levels in supernatants were quantified by the Michigan Regional Comprehensive Metabolomics Resource Core at the University of Michigan, Ann Arbor, Mich., in a delta 4 (D4-) steroid hormone assay using combined ultra-high pressure liquid chromatography and triple quadrapole (UHPLC-QQQ) mass spectrometry. In brief, target steroid analytes were chromatographically separated on a 2.1 mm×50 mm Biphenyl column in a 20 min cycle. All analytes and internal standards were measured by ESI ionization with positive or negative polarity (analyte dependent) on a UHPLC-QQQ mass spectrometer using multiple reaction monitoring (MRM) methods and a 13-point standard curve.

Flow cytometry was performed on a Miltenyi MACSQUANT@ Analyzer 7. T-lymphocyte markers were labeled using conjugated antibodies for CD3 (APC-H7 conjugate) (Human Naive/Memory T Cell Panel, BD Biosciences, San Jose, Calif.). The cell surface tag was detected using supernatant from the Chessie 13-9.1 hybridoma (a murine IgG1 antibody; NIH AIDS Reagent Program, catalog number 1209, lot number 040144, antibody deposited by Dr. George Lewis) followed by staining with a secondary goat-anti-mouse-IgG polyclonal conjugated with Phycoerythrin (PE) (Immunoreagents, Raleigh, N.C.).

Prior to antibody labeling, cells were incubated with an Fc-receptor blocker (Human TRUSTAIN FCX™, BioLegend, Pacific Heights, Calif.). Live versus dead cell identification utilized a dye added after staining (ZOMBIE VIOLET®, BioLegend, Pacific Heights, Calif.). Analysis of marker expression following flow cytometry used FlowJo software (version 10.5.3, FlowJo LLC, Ashland, Oreg.) and selection of events with live, single cells and positive/negative gates set using fluorescence minus one (FMO) and/or secondary only controls.

Longer persistence in peripheral blood compared to non steroid-resistant cells has been achieved in animal models or humans.

### Results

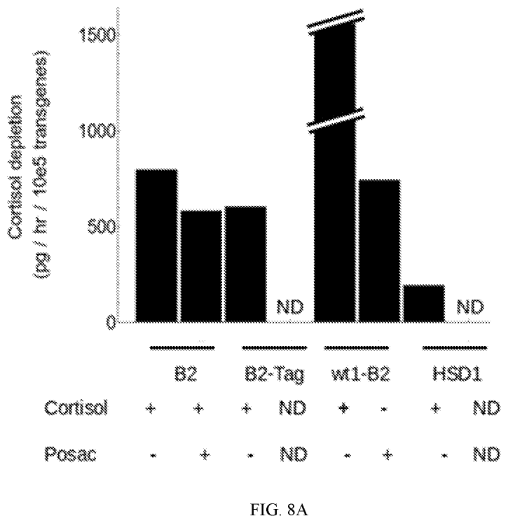

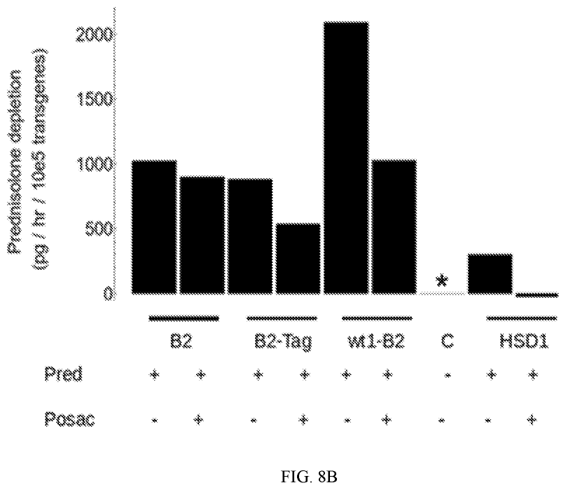

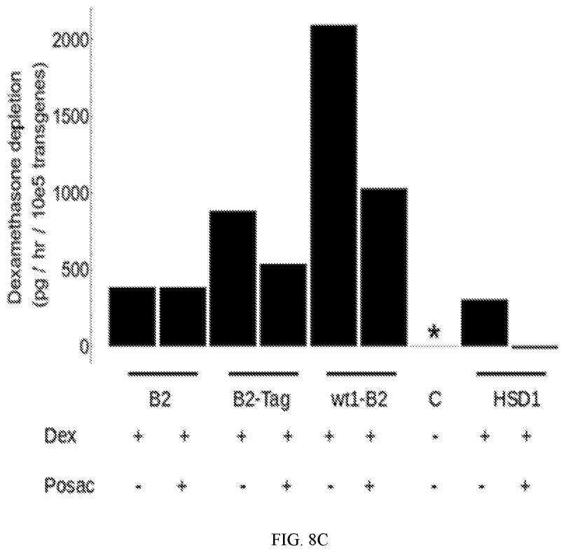

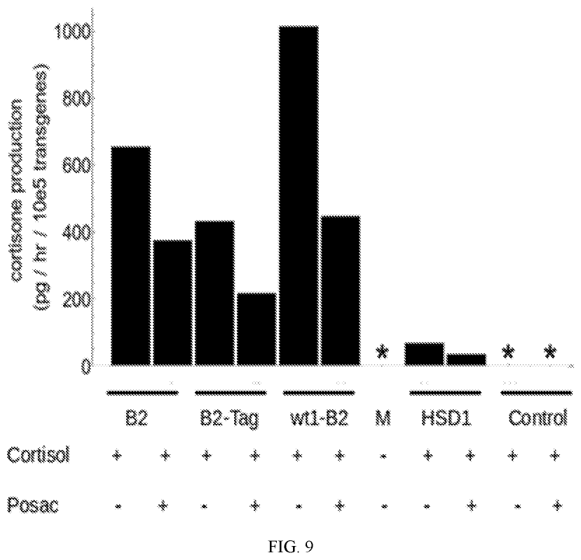

The lentivectors described herein all showed transduction of stimulated leukocytes, measured by significant levels of viral copies (VCN assay). In the case of the B2-Tag construct, transduction was also monitored using flow cytometry detection of a cell surface tag expressed by the B2-Tag construct (FIGS. 8A-8C). In FIG. 8A, with “wt1-B2,” the cortisol levels at end of assay were below the limit of quantitation by the ELISA, indicating all cortisol were depleted and the depletion rate incalculable from this data, hence the broken bar thereof. Since cortisol depletion and cortisone production are directly related, assuming no inter-conversion, in analyzing the cortisone production (measured by UHPLC-Mass Spectrometry in FIG. 9), a rate of cortisol depletion is indirectly calculated to be about 1,014 pg/hr/10e5 copies of the transgene. The “wt1-B2” bar could be assumed to have this height, approximately the location of the lower bar-break icon.

Transduction of stimulated leukocytes with genes encoding HSD11B1 and HSD11B2 conferred the ability to degrade steroids (FIG. 9). With the HSD11B2 genes (HSD11B2, B2-Tag and wtExon1-none) this degradation was inhibited by a known HSD11B2 inhibitor, posaconazole (FIG. 9). HSD11B1 degradation of steroids was also inhibited by posaconazole. Although HSD11B1 displayed a lower rate of depletion (conversion) of cortisol, prednisolone and dexamethasone compared to HSD2 constructs, HSD11B1 depletion (conversion) of non-natural steroids prednisolone and dexamethasone was strongly inhibited by posaconazole.

The HSD11B2 constructs showed distinct properties with respect to enzymatic activity, measured by steroid degradation and conversion, and their sensitivity to the inhibitor posaconazole. In contrast to the codon optimized sequence of HSD11B2, wtExon1-none retains the sequence of exon 1 of a “wild type” HSD11B2 mRNA. Exon 1 of HSD11B2 is a region that may be involved in regulation of the HSD11B2 gene through the action of the antisense transcript AC009061.1 present in the genome on the opposite strand to the HSD11B2 gene and which has been detected to be expressed.

The negative strand of the HDS11B2 gene includes a region that results in detectable transcripts. The transcript form this region has been given various designations including AC009061.1, ENST00000567261.1, ENSG00000261320.1, Locus LF212233 and JP 2014500723-A/19736: Polycomb-Associated Non-Coding RNAs and maps to chromosome region hg38 chr16:67,430,667-67,431,464. The AC009061.1 transcript begins in exon 1 of HSD11B2 and continues as a complementary sequence through the translation start site, the predicted Kozak region and along the 5′ untranslated region of HSD11B2 gene. It has features resembling a long non-coding RNA (lncRNA) which can be found overlapping with regions of the sense strand and promoter regions AC009061.1 has also been classified a member of the Polycomb-Associated Non-Coding RNA family of RNA regulators of gene expression. Whereas RNA complementary to mRNA can result in suppression of gene expression, expression of some genes are stimulated by the presence of complementary sequences (e.g. small activating RNAs; saRNAs).

The bicistronic construct B2-Tag resulted in a B2 protein with additional amino acids on the C-terminal end. These additional amino acids may alter the stability, intracellular location and interaction of the modified HSD11B2 protein with other cell components and proteins.

As shown by the data, the three HSD11B2 constructs and the HSD11B1 construct had distinctive activities and sensitivities to the model HSD inhibitor posaconazole. The tested HSD11B2 constructs are in three distinct forms: i) a fully codon optimized HSD11B2 construct (B2), ii) a construct that was codon optimized but which retained the wild-type exon 1 sequence (wtExon1-none), and iii) a construct which resulted in additional amino acids on the C-terminal end of HSD11B2 (B2-Tag). These observations can be exploited to generate new constructs. Constructs can be generated to have a desired degree of HSD activity and sensitivity to HSD inhibitors by incorporating one or more of the three elements of: codon optimization, wild-type exon 1, and C-terminal extension.

Further modifications of HSD constructs include the incorporation of modifications to the protein sequence that increases or reduces enzyme activity. There are other examples of changes that cause reductions of enzyme activity. Gene evolution and screening of variants can identify HSD genes with increased activity towards substrates as well as sensitivity to, or resistance from, enzyme inhibitors.

It is contemplated that HSD11B1 gene constructs can also be built as bicistronic constructs in the general form of “Desired Gene-HSD11B1” or “HSD11B1-Desired Gene”, that is where a Desired Gene sequence is located at the 5′ or 3′ of the region encoding the HSD11B1 gene and may include intervening self-cleaving sequences such as members of the 2A family.

Poly-cistronic constructs of HSD11B1 and HSD11B2 are also contemplated and readily constructed. By way of example, a bicistronic HSD11B2 gene where construct is of the general form “Desired Gene-2A region-HSD11B2 gene” (Desired Gene is upstream of HSD11B2 gene), that is, where the Desired Gene is a cell surface marker gene located at the 5′ of the HSD11B2 gene and is followed by a cleavable 2A linked which results in modifications to the N-terminus of the HSD11B2 gene following 2A cleavage (“B2-TagBefore,” i.e., “tag” is upstream of HSD11B2, having an exemplary polynucleotide sequence as set forth in SEQ ID No.: 37, which translates to a polypeptide sequence as set forth in SEQ ID No.: 38).

As seen in HEK-293 (human embryonic kidney) cells, modifications of HSD11B1 and HSD11B2 wherein additional amino acid sequences are added to their N- or C-termini can produce HSD11B1 or HSD11B2 proteins that have enzyme activities that are indistinguishable from unmodified enzymes and retain the ability to localize intracellularly. This study is the first study to show that modified HSD11B2 constructs have useful properties when expressed in leukocytes. Sequence related structures have been identified and can be used to create new HSD constructs with improved steroid degradation properties and wherein such constructs also have varying degrees of responsiveness to inhibitors.

In these studies, some exemplary HSD inhibitors such as carbenoxolone and posaconazole are described. Methods are provided to evaluate leukocytes that have been gene modified with a HSD construct for the cells' ability to degrade steroids and the degree to which HSD activity can be reduced by a contemplated inhibitor. In this manner, HSD constructs can be designed, evaluated and then chosen for use based on five general considerations related to their clinical use. These considerations are: i) the dehydrogenase activity of the HSD construct against the anticipated steroid or steroids to be used clinically and where resistance to the steroid(s) is desired, ii) the ability to inhibit the enzyme using an inhibitor compound, preferably a compound that can reach concentrations in the patient that inhibits the HSD and wherein use of the compound is not contraindicated as a result of the patients current or contemplated future medical condition, iii) the ability to “by-pass” the conferred steroid resistance by dosing the patient with a distinct steroid that has been determined to not be a good substrate for the HSD, iv) where there is a requirement or likelihood to use drugs that would be an inhibitor of one HSD construct (e.g. HSD11B2) and instead choose to use an alternate HSD (e.g. HDS1), and v) to choose an alternate drug that is clinically effective for the patients underlying condition (e.g., from the azole class of anti-fungals) but to select a drug (e.g., from a class of drugs) where the drug is chosen with respect to its ability to inhibit the HSD construct being used. By way of an example of consideration (v), when measured using HEK-293 cell lysates with added exogenous NAD, azole fungicides have inhibitory potency against HSD11B2 in the order of (increasing left to right) Albendazole<Climbazole<Tioconazole<Sertaconazole<Butoconazole<Keotconazole<Terconazole<Posaconazole<Itraconazole. This contrasts with HSD11B1 which, when evaluated for HSD11B1 reductase activity in cell lysates in the presence of added NADPH, the order of azole fungicides' inhibitory potency is essentially reversed. Dosing a patient with a drug such as an azole fungicide determined to have low inhibitory activity against an HSD-transduced leukocyte supports treatment of the underlying condition that requires the antifungal and preserves the steroid resistance of the adoptive cell therapy. Dosing the same patient with a drug such as a different azole fungicide determined to have high inhibitory activity against an HSD in a transduced leukocyte allows treatment of the underlying condition and reduction of the HSD's steroid degradation function, thus rendering the immunotherapeutic cells more responsive to steroids.

As such, exemplary and non-limiting embodiments of the invention are demonstrated, considering the three general factor for HSD selection, with different sequence modifications and the use of “by-pass” steroids or enzyme inhibitors.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A population of genetically modified leukocytes wherein at least ten percent of the genetically modified leukocytes express a gene that confers resistance to a glucocorticoid.

2. The population of genetically modified leukocytes of claim 1, wherein the gene that confers resistant to a glucocorticoid is selected from a group consisting of 11-beta-hydroxysteroid dehydrogenase type II (HSD11B2), 11-beta-hydroxysteroid dehydrogenase type I (HSD11B1), and a combination thereof.

3. The population of genetically modified leukocytes of claim 1, wherein the gene encodes corticosteroid 11-beta-dehydrogenase isozyme 2 or the gene comprises a polynucleotide sequence set forth in any one of SEQ ID Nos.: 31, 18 and 32, wherein the 11-beta-dehydrogenase isozyme 2 comprises a polypeptide sequence set forth in any one of SEQ ID Nos.: 3, 33 and 38.

4. (canceled)

5. (canceled)

6. The population of genetically modified leukocytes of claim 2, wherein the gene encodes corticosteroid 11-beta-dehydrogenase isozyme 1 or the gene comprises a polynucleotide sequence set forth in SEQ ID No.:17, and the leukocytes comprises lymphocytes.

7. (canceled)

8. The population of genetically modified leukocytes of claim 1, wherein the genetically modified leukocytes comprise a first vector comprising the gene that confers resistance to a glucocorticoid, and the genetically modified leukoeytes further comprise a genetic modification to provide a therapeutic effect for adoptive cell transfer.

9. (canceled)

10. The population of genetically modified leukocytes of claim 1, wherein the gene that confers resistance to a glucocorticoid is transfected into leukocytes to form the genetically modified leukocytes, and the leukocytes are stimulated with an antigen before the transfection.

11. The population of genetically modified leukocytes of claim 1, wherein the gene that confers resistance to a glucocorticoid is transfected into leukocytes to form the genetically modified leukocytes, and the leukocytes are stimulated with an antigen after the transfection.

12. The population of genetically modified leukocytes of claim 1, wherein the leukocytes are selected from the group consisting of cytotoxic T-cells, helper T-cells, large granular lymphocytes, leukocyte precursors, lymphocytes, mast cells, memory cells, natural killer cells, natural killer T cells, regulatory T-cells (Tregs), suppressor T-cells, T-cells, tumor infiltrating lymphocytes, and a combination thereof.

13. A pharmaceutical composition comprising a population of genetically modified leukocytes of claim 1, and a pharmaceutically acceptable carrier or diluent.

14. A method of modulating steroid resistance of immune cells in a mammalian subject in need thereof, comprising:
administering a therapeutically effective amount of the pharmaceutical composition of claim 13 to increase resistance to steroid of the immune cells in the subject;
and optionally further administering an effective amount of an inhibitor of 11-beta-hydroxysteroid dehydrogenase to the subject to reduce steroid resistance of the immune cells in the subject.

15. The method of claim 14, wherein the inhibitor of 11-beta-hydroxysteroid dehydrogenase comprises carbenoxolone, itraconazole, hydroxyitraconazole, ketaconazole, or posaconazole.

16. The method of claim 15, wherein the inhibitor of 11-beta-hydroxysteroid dehydrogenase is carbenoxolone.

17. The method of claim 14, wherein the subject is administered with a therapy selected from the group consisting of glucocorticoid, a nonsteroidal anti-inflammatory drug, an anti-infective, and a chemotherapeutic agent.

18. The method of claim 14, wherein the genetically modified leukocytes of the pharmaceutical composition are further modified to express a recombinant T-cell receptor or a chimeric T cell antigen receptor.

19. A method of treating or reducing the likelihood of a cancer, an infection, or an auto-immune disorder in a patient in need thereof comprising:
administering a therapeutically effective amount of a population of genetically modified leukocytes according to claim 1.

20. (canceled)

21. (canceled)

22. A method of improving the in vitro growth of genetically modified leukocytes of claim 1, said leukocytes expressing an HSD11B2 gene, comprising
incubating said leukocytes with an effective amount of an inhibitor of HSD11B2 activity.

23. A method of screening an inhibitor capable of reversing glucocorticoid resistance, comprising:
contacting an effective amount of a candidate agent with a population of cells including at least five percent genetically modified leukocytes that express a gene that confers resistance to 11-beta-hydroxysteroids;
measuring resistance to steroids of the population of cells; and
identifying the candidate agent as an inhibitor capable of reversing glucocorticoid resistance when a loss or reduction of resistance to steroids of the population of cells is measured and identifying the candidate agent is not an inhibitor capable of reversing glucocorticoid resistance when no loss or reduction of resistance is measured.

24. An expression vector comprising a gene that encodes a protein which confers resistance to a glucocorticoid, wherein the vector comprises a polynucleotide sequence set forth in any one of SEQ ID Nos. 31, 32, 37, 18, 17 and 19-29.

25. The expression vector of claim 24, further comprising a backbone of SEQ ID NO:30.

26. A method of producing a population of genetically modified leukocytes, comprising electroporating leukocytes with the expression vector of claim 24 to produce the genetically modified leukocytes.


SEQUENCE LISTING (46 sequences)

SEQ ID NO: 1 - protein, 383 aa, Artificial Sequence
  synthetic construct
MDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 2 - protein, 405 aa, Homo sapiens
MPLLLYTCLLWLPTSGLWTVQAMDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 3 - protein, 405 aa, Homo sapiens
MERWPWPSGGAWLLVAARALLQLLRSDLRLGRPLLAALALLAALDWLCQRLLPPPAALAVLAAAGWIALSRLARPQRLPVATRAVLITGCDSGFGKETAKKLDSMGFTVLATVLELNSPGAIELRTCCSPRLRLLQMDLTKPGDISRVLEFTKAHTTSTGLWGLVNNAGHNEVVADAELSPVATFRSCMEVNFFGALELTKGLLPLLRSSRGRIVTVGSPAGDMPYPCLGAYGTSKAAVALLMDTFSCELLPWGVKVSIIQPGCFKTESVRNVGQWEKRKQLLLANLPQELLQAYGKDYIEHLHGQFLHSLRLAMSDLTPVVDAITDALLAARPRRRYYPGQGLGLMYFIHYYLPEGLRRRFLQAFFISHCLPRALQPGQPGTTPPQDAAQDPNLSPGPSPAVAR

SEQ ID NO: 4 - protein, 292 aa, Homo sapiens
MAFMKKYLLPILGLFMAYYYYSANEEFRPEMLQGKKVIVTGASKGIGREMAYHLAKMGAHVVVTARSKETLQKVVSHCLELGAASAHYIAGTMEDMTFAEQFVAQAGKLMGGLDMLILNHITNTSLNLFHDDIHHVRKSMEVNFLSYVVLTVAALPMLKQSNGSIVVVSSLAGKVAYPMVAAYSASKFALDGFFSSIRKEYSVSRVNVSITLCVLGLIDTETAMKAVSGIVHMQAAPKEECALEIIKGGALRQEEVYYDSSLWTTLLIRNPCRKILEFLYSTSYNMDRFINK

SEQ ID NO: 5 - protein, 234 aa, Homo sapiens
MKVLLLTGLGALFFAYYWDDNFDPGGLDYLVLNHIGGAPAGTRARSPQATRWLMQVNFVSYVQLTSRALPSLTDSKGSLVVVSSLLGRVPTSFSTPYSAAKFALDGFFGSLRRELDVQDVNVAITMCVLGLRDRASAAEAVRSSTSRPRQPEHRGVPLQSQTAMFLPPTVPGARTLTETPLRGWPQPKMKSSRQKSKTEKNDGHLEPVTAWEVQVPRVRRLCRGLARPHLFGHD

SEQ ID NO: 6 - protein, 204 aa, Homo sapiens
MKVLLLTGLGALFFAYYWDDNFDPGKLCELRATDVAGAAQPDGQQGLPGGGVLAARPRAHVVLHSLLGGQVCAGRLLRLPAAGAGRAGRERGHHHVRPGPPRSRLRRRGSQGSHEGQGGPGAQGSPGRDPRRRHARGRRLLPVAFPPAVLAPALATAPAGLVYPPGAQRHGRGSLSTGGCPSSPRRQCSSLQLSLEPEHSQRHP

SEQ ID NO: 7 - protein, 315 aa, Homo sapiens
MKVLLLTGLGALFFAYYWDDNFDPASLQGARVLLTGANAGVGEELAYHYARLGSHLVLTAHTEALLQKVVGNCRKLGAPKVFYIAADMASPEAPESVVQFALDKLGGLDYLVLNHIGGAPAGTRARSPQATRWLMQVNFVSYVQLTSRALPSLTDSKGSLVVVSSLLGRVPTSFSTPYSAAKFALDGFFGSLRRELDVQDVNVAITMCVLGLRDRASAAEAVRSSTSRPRQPEHRGVPLQSQTAMFLPPTVPGARTLTETPLRGWPQPKMKSSRQKSKTEKNDGHLEPVTAWEVQVPRVRRLCRGLARPHLFGHD

SEQ ID NO: 8 - protein, 333 aa, Homo sapiens
MANLGTLQLLPPRFKRFSCLSLPNIWITGMPVPATSVPCPSAGPHRTMKVLLLTGLGALFFAYYWDDNFDPASLQGARVLLTGANAGVGEELAYHYARLGSHLVLTAHTEALLQKVVGNCRKLGAPKVFYIAADMASPEAPESVVQFALDKLGGLDYLVLNHIGGAPAGTRARSPQATRWLMQVNFVSYVQLTSRALPSLTDSKGSLVVVSSLLGRVPTSFSTPYSAAKFALDGFFGSLRRELDVQDVNVAITMCVLGLRDRASAAEAVRGVTRVKAAPGPKAALAVIRGGATRAAGVFYPWRFRLLCLLRRWLPRPRAWFIRQELNVTAAAA

SEQ ID NO: 9 - protein, 286 aa, Homo sapiens
MKVLLLTGLGALFFAYYWDDNFDPASLQGARVLLTGANAGVGEELAYHYARLGSHLVLTAHTEALLQKVVGNCRKLGAPKVFYIAADMASPEAPESVVQFALDKLGGLDYLVLNHIGGAPAGTRARSPQATRWLMQVNFVSYVQLTSRALPSLTDSKGSLVVVSSLLGRVPTSFSTPYSAAKFALDGFFGSLRRELDVQDVNVAITMCVLGLRDRASAAEAVRGVTRVKAAPGPKAALAVIRGGATRAAGVFYPWRFRLLCLLRRWLPRPRAWFIRQELNVTAAAA

SEQ ID NO: 10 - protein, 152 aa, Homo sapiens
MQVNFVSYVQLTSRALPSLTDSKGSLVVVSSLLGRVPTSFSTPYSAAKFALDGFFGSLRRELDVQDVNVAITMCVLGLRDRASAAEAVRGVTRVKAAPGPKAALAVIRGGATRAAGVFYPWRFRLLCLLRRWLPRPRAWFIRQELNVTAAAA

SEQ ID NO: 11 - protein, 181 aa, Homo sapiens
MQVNFVSYVQLTSRALPSLTDSKGSLVVVSSLLGRVPTSFSTPYSAAKFALDGFFGSLRRELDVQDVNVAITMCVLGLRDRASAAEAVRSSTSRPRQPEHRGVPLQSQTAMFLPPTVPGARTLTETPLRGWPQPKMKSSRQKSKTEKNDGHLEPVTAWEVQVPRVRRLCRGLARPHLFGHD

SEQ ID NO: 12 - protein, 199 aa, Homo sapiens
MASPEAPESVVQFALDKLGGLDYLVLNHIGGAPAGTRARSPQATRWLMQVNFVSYVQLTSRALPSLTDSKGSLVVVSSLLGRVPTSFSTPYSAAKFALDGFFGSLRRELDVQDVNVAITMCVLGLRDRASAAEAVRGVTRVKAAPGPKAALAVIRGGATRAAGVFYPWRFRLLCLLRRWLPRPRAWFIRQELNVTAAAA

SEQ ID NO: 13 - protein, 205 aa, Homo sapiens
MKVLLLTGLGALFFAYYWDDNFDPGGLDYLVLNHIGGAPAGTRARSPQATRWLMQVNFVSYVQLTSRALPSLTDSKGSLVVVSSLLGRVPTSFSTPYSAAKFALDGFFGSLRRELDVQDVNVAITMCVLGLRDRASAAEAVRGVTRVKAAPGPKAALAVIRGGATRAAGVFYPWRFRLLCLLRRWLPRPRAWFIRQELNVTAAAA

SEQ ID NO: 14 - protein, 286 aa, Homo sapiens
MKVLLLTGLGALFFAYYWDDNFDPASLQGARVLLTGANAGVGEELAYHYARLGSHLVLTAHTEALLQKVVGNCRKLGAPKVFYIAADMASPEAPESVVQFALDKLGEGLGLNPGVRDRGLGLRDRTRIGLWCRLQVNFVSYVQLTSRALPSLTDSKGSLVVVSSLLGRVPTSFSTPYSAAKFALDGFFGSLRRELDVQDVNVAITMCVLGLRDRASAAEAVRGVTRVKAAPGPKAALAVIRGGATRAAGVFYPWRFRLLCLLRRWLPRPRAWFIRQELNVTAAAAA

SEQ ID NO: 15 - DNA, 20 bp, Artificial Sequence
  synthetic construct
ccaaggacctgaaatgaccc

SEQ ID NO: 16 - DNA, 32 bp, Artificial Sequence
  synthetic construct
ctgaataataagatgacatgaactactactgc

SEQ ID NO: 17 - DNA, 970 bp, Artificial Sequence
  synthetic construct
cccctcactcggcgcgatctagatctcgaatcgccctgtcggatggcttttatgaaaaaatatctcctccccattctggggctcttcatggcctactactactattctgcaaacgaggaattcagaccagagatgctccaaggaaagaaagtgattgtcacaggggccagcaaagggatcggaagagagatggcttatcatctggcgaagatgggagcccatgtggtggtgacagcgaggtcaaaagaaactctacagaaggtggtatcccactgcctggagcttggagcagcctcagcacactacattgctggcaccatggaagacatgaccttcgcagagcaatttgttgcccaagcaggaaagctcatgggaggactagacatgctcattctcaaccacatcaccaacacttctttgaatctttttcatgatgatattcaccatgtgcgcaaaagcatggaagtcaacttcctcagttacgtggtcctgactgtagctgccttgcccatgctgaagcagagcaatggaagcattgttgtcgtctcctctctggctgggaaagtggcttatccaatggttgctgcctattctgcaagcaagtttgctttggatgggttcttctcctccatcagaaaggaatattcagtgtccagggtcaatgtatcaatcactctctgtgttcttggcctcatagacacagaaacagccatgaaggcagtttctgggatagtccatatgcaagcagctccaaaggaggaatgtgccctggagatcatcaaagggggagctctgcgccaagaagaagtgtattatgacagctcactctggaccactcttctgatcagaaatccatgcaggaagatcctggaatttctctactcaacgagctataatatggacagattcataaacaagtagcctgaaaaaggggtacctttaagaccaatgacttacaaggcagctgtag

SEQ ID NO: 18 - DNA, 1318 bp, Artificial Sequence
  synthetic construct
cccctcactcggcgcgatctagatctcgaatcgccagcccgctgggccgccatggagcgttggccttggccatcgggtggtgcttggctgctcgtggctgctcgtgcactgctgcagctgctgcgttcagacctgcgtctgggtcgtccactgctggcagcactggcactgctggctgcactcgactggctgtgccagcgtctgctgcctccaccagctgcactcgctgtgctggctgctgctggttggatcgcattgtcccgtctggcacgtccacagcgtctgccagtggctactcgtgcagtgctcatcaccggttgtgactctggttttggtaaggagacggctaagaaactggactccatgggtttcacggtgctggctaccgtattggagttgaacagccctggtgctatcgagctgcgtacctgctgctcccctcgtctaaggctgctgcagatggacctgaccaaaccaggagacattagccgtgtgctagagttcaccaaggctcacaccaccagcaccggtctgtggggtctcgtcaacaacgcaggtcacaatgaagtagttgctgatgcagagctgtctccagtggctactttccgtagctgcatggaggtgaatttctttggtgcactcgagctgaccaagggtctcctgcctctgctgcgtagctcaaggggtcgtatcgtgactgtgggaagcccagcaggagacatgccatatccatgcttgggagcttatggaacctccaaagcagctgtggcactactcatggacacattcagctgtgaactccttccttggggagtcaaggtcagcatcatccagcctggttgcttcaagacagagtcagtgagaaacgtgggtcagtgggaaaagcgtaagcaattgctgctggctaacctgcctcaagagctgctgcaggcttacggtaaggactacatcgagcacttgcatggacagttcctgcactcgctacgtctggctatgtccgacctcaccccagttgtagatgctatcacagatgcactgctggcagctaggcctcgtcgtcgttattaccctggtcagggtctgggactcatgtacttcatccactactacctgcctgaaggtctgaggcgtcgtttcctgcaggctttcttcatcagtcactgtctgcctcgagcactgcagcctggtcagcctggtactaccccaccacaggacgcagctcaggacccaaacctgagccctggtccttccccagcagtggctaggtgacctgaaaaaggggtacctttaagaccaatgacttacaaggcagctgtag

SEQ ID NO: 19 - DNA, 938 bp, Artificial Sequence
  synthetic construct
cccctcactcggcgcgatctagatctcgaatcgaggaccatgaaggtgcttctcctcacaggactgggagctctgttcttcgcttattattgggatgacaactttgacccagctagcctccagggagcacgagtgctgctgacaggagctaatgctggtgttggtgaggagctggcttatcactacgcacgtctgggttcccacctggtgctcactgctcacactgaggctctcctgcagaaggtggtaggaaactgcaggaagctgggtgctcctaaggtcttctacatcgcagcagacatggcttcccctgaggcacctgagagcgtggtgcagtttgcactggacaagctgggtgagggactgggtctgaatcctggagtcagggaccgtggtctaggtcttagggacaggaccagaattggactgtggtgccgtctgcaggtaaactttgtgagctacgtgcaactgacgtcgagggcactgcctagcctgacagacagcaagggttccctggtggtggtgtcctcgctgctcggtcgtgtgcctacgtcgttctccactccatactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggggagtcacgagggtcaaggcagctccaggacctaaggcagctctggctgtgatccgtggtggtgctacgcgtgcagctggtgtcttctacccatggcgtttccgtctgctgtgcttgctcaggcgttggctgccacgtccaagggcttggtttatccgtcaggagctcaacgtcacggctgcagctgcagcttgaggtacctttaagaccaatgacttacaaggcagctgtag

SEQ ID NO: 20 - DNA, 1079 bp, Artificial Sequence
  synthetic construct
cccctcactcggcgcgatctagatctcgaatcgaggaccatggcaaatctcggtacactacaacttctgcctcctaggttcaagcgattctcctgcctcagcctcccaaatatctggattacaggtatgcctgtgccagctacctctgtcccttgtccttctgcaggtccacacaggaccatgaaggtgcttctcctcacaggactgggagctctgttcttcgcttattattgggatgacaacttcgacccagctagcctccagggagcacgagtgctgctgacaggagctaacgctggtgttggtgaggagctggcttatcactacgcacgtctgggttcccacctggtgctcactgctcacactgaggctctcctgcagaaggtggtaggaaactgcaggaagctgggtgctcctaaggtcttctacatcgcagcagacatggcttcccctgaggcacctgagagcgtggtgcagtttgcactggacaagctgggtggactggactacctcgtgctgaaccacatcggtggtgctccagctggtacgcgagctcgtagccctcaggcaactcgttggctcatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggggagtcacgagggtcaaggcagctccaggacctaaggcagctctggctgtgatccgtggtggtgctacgcgtgcagctggtgtcttctacccatggcgtttccgtctgctgtgcttgctcaggcgttggctaccacgtccaagggcttggtttatccgtcaggagctcaacgtcacggctgcagcagcttgaggtacctttaagaccaatgacttacaaggcagctgtag

SEQ ID NO: 21 - DNA, 938 bp, Artificial Sequence
  synthetic construct
cccctcactcggcgcgatctagatctcgaatcgaggaccatgaaggtgcttctcctcacaggactgggagctctgttcttcgcttattattgggatgacaacttcgacccagctagcctccagggagcacgagtgctgctgacaggagctaacgctggtgttggtgaggagctggcttatcactacgcacgtctgggttcccacctggtgctcactgctcacactgaggctctcctgcagaaggtggtaggaaactgcaggaagctgggtgctcctaaggtcttctacatcgcagcagacatggcttcccctgaggcacctgagagcgtggtgcagtttgcactggacaagctgggtggactggactacctcgtgctgaaccacatcggtggtgctccagctggtacgcgagctcgtagccctcaggcaactcgttggctcatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggggagtcacgagggtcaaggcagctccaggacctaaggcagctctggctgtgatccgtggtggtgctacgcgtgcagctggtgtcttctacccatggcgtttccgtctgctgtgcttgctcaggcgttggctaccacgtccaagggcttggtttatccgtcaggagctcaacgtcacggctgcagcagcttgaggtacctttaagaccaatgacttacaaggcagctgtag

SEQ ID NO: 22 - DNA, 688 bp, Artificial Sequence
  synthetic construct
cccctcactcggcgcgatctagatctcgaatcgaggaccatggcttcccctgaggcacctgagagcgtggtgcagtttgcactggacaagctgggtggactggactacctcgtgctgaaccacatcggtggtgctccagctggtacgcgagctcgtagccctcaggcaactcgttggctcatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggggagtcacgagggtcaaggcagctccaggacctaaggcagctctggctgtgatccgtggtggtgctacgcgtgcagctggtgtcttctacccatggcgtttccgtctgctgtgcttgctcaggcgttggctaccacgtccaagggcttggtttatccgtcaggagctcaacgtcacggctgcagcagcttgacctgaaaaaggggtacctttaagaccaatgacttacaaggcagctgtag

SEQ ID NO: 23 - DNA, 547 bp, Artificial Sequence
  synthetic construct
cccctcactcggcgcgatctagatctcgaatcgaggaccatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggggagtcacgagggtcaaggcagctccaggacctaaggcagctctggctgtgatccgtggtggtgctacgcgtgcagctggtgtcttctacccatggcgtttccgtctgctgtgcttgctcaggcgttggctaccacgtccaagggcttggtttatccgtcaggagctcaacgtcacggctgcagcagcttgacctgaaaaaggggtacctttaagaccaatgacttacaaggcagctgtag

SEQ ID NO: 24 - DNA, 1025 bp, Artificial Sequence
  synthetic construct
cccctcactcggcgcgatctagatctcgaatcgaggaccatgaaggtgcttctcctcacaggactgggagctctgttcttcgcttattattgggatgacaacttcgacccagctagcctccagggagcacgagtgctgctgacaggagctaacgctggtgttggtgaggagctggcttatcactacgcacgtctgggttcccacctggtgctcactgctcacactgaggctctcctgcagaaggtggtaggaaactgcaggaagctgggtgctcctaaggtcttctacatcgcagcagacatggcttcccctgaggcacctgagagcgtggtgcagtttgcactggacaagctgggtggactggactacctcgtgctgaaccacatcggtggtgctccagctggtacgcgagctcgtagccctcaggcaactcgttggctcatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggagctcaacgtcaaggccaaggcagcctgagcacaggggagtgcctctccagtcccagacggcaatgttcctccctccaactgtccctggagctagaacactcacagagacacctctgagaggatggccacagcctaagatgaagtcatcaagacagaaaagcaaaaccgagaaaaacgacggacacctggaaccagtcacggcttgggaggtgcaggtgcctcgtgttaggcgtctttgtaggggacttgcaaggcctcacctgtttggtcatgattgaggtacctttaagaccaatgacttacaaggcagctgtag

SEQ ID NO: 25 - DNA, 706 bp, Artificial Sequence
  synthetic construct
cccctcactcggcgcgatctagatctcgaatcgaggaccatgaaggtgcttctcctcacaggactgggagctctgttcttcgcttattattgggatgacaacttcgacccaggtggactggactacctcgtgctgaaccacatcggtggtgctccagctggtacgcgagctcgtagccctcaggcaactcgttggctcatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggggagtcacgagggtcaaggcagctccaggacctaaggcagctctggctgtgatccgtggtggtgctacgcgtgcagctggtgtcttctacccatggcgtttccgtctgctgtgcttgctcaggcgttggctaccacgtccaagggcttggtttatccgtcaggagctcaacgtcacggctgcagcagcttgacctgaaaaaggggtacctttaagaccaatgacttacaaggcagctgtag

SEQ ID NO: 26 - DNA, 692 bp, Artificial Sequence
  synthetic construct
cccctcactcggcgcgatctagatctcgaatcgaggaccatgaaggtgcttctcctcacaggactgggagctctgttcttcgcttattattgggatgacaacttcgacccaggtaaactttgtgagctacgtgcaactgacgtcgcaggtgctgctcagcctgacggacagcaaggactccctggtggtggtgtcctcgctgctaggccacgtgctcacgtcgttctccactccctactcggtggtcaagtttgcgctggaaggcttcttaggctccctgcagcaggagctggacgtgcaggacgtgaacgtggtcatcaccatgtgcgtcctggacctccaagatcgcgtctccgtcgtcgaggtagtcagggaagtcacgagggtcaaggtggtcctggagctcaaggtagccctggtcgtgatccaaggaggcgtcacgcacgtggtaggcgtcttctacctgtggcatttccacctgctgtgcttgctccagcactggctaccgcacctgcaggtctggtttatccaccaggagctcaacgtcacggtcgtggtagcctgagcaccggaggatgcccttccagtcctagaaggcaatgttcctccctccaactgtccctggagccagaacactcacagagacacccttgaggtacctttaagaccaatgacttacaaggcagctgtag

SEQ ID NO: 27 - DNA, 623 bp, Artificial Sequence
  synthetic construct
cccctcactcggcgcgatctagatctcgaatcgaggaccatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggagctcaacgtcaaggccaaggcagcctgagcacaggggagtgcctctccagtcccagacggcaatgttcctccctccaactgtccctggagctagaacactcacagagacacctctgagaggatggccacagcctaagatgaagtcatcaagacagaaaagcaaaaccgagaaaaacgacggacacctggaaccagtcacggcttgggaggtgcaggtgcctcgtgttaggcgtctttgtaggggacttgcaaggcctcacctgtttggtcatgattgaggtacctttaagaccaatgacttacaaggcagctgtag

SEQ ID NO: 28 - DNA, 782 bp, Artificial Sequence
  synthetic construct
cccctcactcggcgcgatctagatctcgaatcgaggaccatgaaggtgcttctcctcacaggactgggagctctgttcttcgcttattattgggatgacaacttcgacccaggtggactggactacctcgtgctgaaccacatcggtggtgctccagctggtacgcgagctcgtagccctcaggcaactcgttggctcatgcaggtaaactttgtgagctacgtgcaactgacgtcgagggcactgcctagcctgacggacagcaagggttccctggtggtggtgtcctcgctgctcggtcgtgtgcctacgtcgttctccactccttactcggcagctaagtttgcactggacggtttcttcggttccctgaggagggagctggacgtgcaggacgtgaacgtggctatcaccatgtgcgtcctgggtctccgagatcgtgcttccgctgctgaggcagtcaggagctcaacgtcaaggccaaggcagcctgagcacaggggagtgcctctccagtcccagacggcaatgttcctccctccaactgtccctggagctagaacactcacagagacacctctgagaggatggccacagcctaagatgaagtcatcaagacagaaaagcaaaaccgagaaaaacgacggacacctggaaccagtcacggcttgggaggtgcaggtgcctcgtgttaggcgtctttgtaggggacttgcaaggcctcacctgtttggtcatgattgaggtacctttaagaccaatgacttacaaggcagctgtag

SEQ ID NO: 29 - DNA, 1246 bp, Artificial Sequence
  synthetic construct
cccctcactcggcgcgatctagatctcgaatcgctatactggacaatggatcctaacgctgcttatgtgaacatgagtaaccatcacaggggtctggcttcagctaacgttgactttgctttcagcctgtataagcacctagtggctttgagtcctaaaaagaacattttcatctcccctgtgagcatctccatggctttagctatgctgtccctgggtacctgtggtcacacaagggctcagcttctccagggtctgggtttcaacctcactgagaggtctgagactgagatccaccagggtttccagcacctgcaccaactctttgcaaagtcagacaccagcttagaaatgaccatgggtaatgctttgtttcttgatggtagcctggagttgctggagtcattctcagcagacatcaagcactactatgagtcagaggtcttggctatgaatttccaggactgggcaacagctagcagacagatcaacagctatgtcaagaataagacacagggaaaaattgtcgacttgttttcaggactggatagcccagctatcctcgtcctggtcaactatatcttcttcaaaggtacatggacacagccttttgacctggcaagcaccagggaggagaacttctatgtggacgagacaactgtggtgaaggtgcctatgatgttgcagtcgagcaccatcagttaccttcatgacgcagagctcccttgccagctggtgcagatgaactacgtgggtaatggaactgtcttcttcatccttccagacaagggaaagatgaacacagtcatcgctgcactgagcagggacacgattaacaggtggtccgcaggtctgaccagcagccaggtggacctgtacattccaaaggtcaccatctctggagtctatgacctcggagatgtgctggaggaaatgggtattgcagacttgttcaccaaccaggcaaatttctcacgtatcacccaggacgctcagctgaagtcatcaaaggtggtccataaagctgtgctgcaactcaatgaggagggtgtggacacagctggttccactggagtcaccctaaacctgacgtccaagcctatcatcttgcgtttcaaccagcctttcatcatcatgatcttcgaccacttcacctggagcagccttttcctggcaagggttatgaacccagtgtaacctgaaaaaggggtacctttaagaccaatgacttacaaggcagctgtag

SEQ ID NO: 30 - DNA, 6571 bp, Artificial Sequence
  synthetic construct
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 31 - DNA, 1318 bp, Artificial Sequence
  wtExon1-none
cccctcactcggcgcgatctagatctcgaatcgccagcccgctgggccgccatggagcgctggccttggccgtcgggcggcgcctggctgctcgtggctgcccgcgcgctgctgcagctgctgcgctcagacctgcgtctgggccgcccgctgctggcggcgctggcgctgctggccgcgctcgactggctgtgccagcgcctgctgcccccgccggccgcactcgccgtgctggccgccgccggctggatcgcgttgtcccgcctggcgcgcccgcagcgcctgccggtggccactcgcgcggtgctcatcaccggttgtgactctggttttggtaaggagacggctaagaaactggactccatgggtttcacggtgctggctaccgtattggagttgaacagccctggtgctatcgagctgcgtacctgctgctcccctcgtctaaggctgctgcagatggacctgaccaaaccaggagacattagccgtgtgctagagttcaccaaggctcacaccaccagcaccggtctgtggggtctcgtcaacaacgcaggtcacaatgaagtagttgctgatgcagagctgtctccagtggctactttccgtagctgcatggaggtgaatttctttggtgcactcgagctgaccaagggtctcctgcctctgctgcgtagctcaaggggtcgtatcgtgactgtgggaagcccagcaggagacatgccatatccatgcttgggagcttatggaacctccaaagcagctgtggcactactcatggacacattcagctgtgaactccttccttggggagtcaaggtcagcatcatccagcctggttgcttcaagacagagtcagtgagaaacgtgggtcagtgggaaaagcgtaagcaattgctgctggctaacctgcctcaagagctgctgcaggcttacggtaaggactacatcgagcacttgcatggacagttcctgcactcgctacgtctggctatgtccgacctcaccccagttgtagatgctatcacagatgcactgctggcagctaggcctcgtcgtcgttattaccctggtcagggtctgggactcatgtacttcatccactactacctgcctgaaggtctgaggcgtcgtttcctgcaggctttcttcatcagtcactgtctgcctcgagcactgcagcctggtcagcctggtactaccccaccacaggacgcagctcaggacccaaacctgagccctggtccttccccagcagtggctaggtgacctgaaaaaggggtacctttaagaccaatgacttacaaggcagctgtag

SEQ ID NO: 32 - DNA, 1962 bp, Artificial Sequence
  B2-Tag After“ or ”tag“ downstream of B2, including overlap with vector
cccctcactcggcgcgatctagatctcgaatcgccagcccgctgggccgccatggagcgttggccttggccatcgggtggtgcttggctgctcgtggctgctcgtgcactgctgcagctgctgcgttcagacctgcgtctgggtcgtccactgctggcagcactggcactgctggctgcactcgactggctgtgccagcgtctgctgcctccaccagctgcactcgctgtgctggctgctgctggttggatcgcattgtcccgtctggcacgtccacagcgtctgccagtggctactcgtgcagtgctcatcaccggttgtgactctggttttggtaaggagacggctaagaaactggactccatgggtttcacggtgctggctaccgtattggagttgaacagccctggtgctatcgagctgcgtacctgctgctcccctcgtctaaggctgctgcagatggacctgaccaaaccaggagacattagccgtgtgctagagttcaccaaggctcacaccaccagcaccggtctgtggggtctcgtcaacaacgcaggtcacaatgaagtagttgctgatgcagagctgtctccagtggctactttccgtagctgcatggaggtgaatttctttggtgcactcgagctgaccaagggtctcctgcctctgctgcgtagctcaaggggtcgtatcgtgactgtgggaagcccagcaggagacatgccatatccatgcttgggagcttatggaacctccaaagcagctgtggcactactcatggacacattcagctgtgaactccttccttggggagtcaaggtcagcatcatccagcctggttgcttcaagacagagtcagtgagaaacgtgggtcagtgggaaaagcgtaagcaattgctgctggctaacctgcctcaagagctgctgcaggcttacggtaaggactacatcgagcacttgcatggacagttcctgcactcgctacgtctggctatgtccgacctcaccccagttgtagatgctatcacagatgcactgctggcagctaggcctcgtcgtcgttattaccctggtcagggtctgggactcatgtacttcatccactactacctgcctgaaggtctgaggcgtcgtttcctgcaggctttcttcatcagtcactgtctgcctcgagcactgcagcctggtcagcctggtactaccccaccacaggacgcagctcaggacccaaacctgagccctggtccttccccagcagtggctaggggcagcggtgaaggacgcggttcactcctcacgtgtggcgatgtggaagagaatccaggtccaggctctggggctactaacttcagccttcttaaacaggcgggagacgttgagaaccctggacctatgcttttgctggtgacttcccttctgctttgcgaactgcctcatccggcctttctcctgatcccccgcaggcccgtcgtgagcacccaactgttgctgaatggttcactggctgaggaggaggtcgtgatacggccagccgagcttcctacacaaggcacgttcagcaacgtcagcacgaatgtcagtcctgcgccacggcctcctaccccagcgccaaccatagcaagtcaaccgctcagcttgagaccagctgcatgcagacccgcagctggtggagctgtacatacacgaggccttgactttgcgtgtgatatttatatctgggcacccttggcagggacgtgtggcgtcctgctgctgtccctcgtaattacgctctactgcaaccacagaaaccgaaggagggtatgcaaatgtccacggcccgttgtctgacctgaaaaaggggtacctttaagaccaatgacttacaaggcagctgtagatcttagccactttttaaaagaaaaggggggactggaagggctaattcactcccaacgaagacaagatctgctttttgcttgt

SEQ ID NO: 33 - protein, 592 aa, Artificial Sequence
  translated ”B2-Tag After“
MERWPWPSGGAWLLVAARALLQLLRSDLRLGRPLLAALALLAALDWLCQRLLPPPAALAVLAAAGWIALSRLARPQRLPVATRAVLITGCDSGFGKETAKKLDSMGFTVLATVLELNSPGAIELRTCCSPRLRLLQMDLTKPGDISRVLEFTKAHTTSTGLWGLVNNAGHNEVVADAELSPVATFRSCMEVNFFGALELTKGLLPLLRSSRGRIVTVGSPAGDMPYPCLGAYGTSKAAVALLMDTFSCELLPWGVKVSIIQPGCFKTESVRNVGQWEKRKQLLLANLPQELLQAYGKDYIEHLHGQFLHSLRLAMSDLTPVVDAITDALLAARPRRRYYPGQGLGLMYFIHYYLPEGLRRRFLQAFFISHCLPRALQPGQPGTTPPQDAAQDPNLSPGPSPAVARGSGEGRGSLLTCGDVEENPGPGSGATNFSLLKQAGDVENPGPMLLLVTSLLLCELPHPAFLLIPRRPVVSTQLLLNGSLAEEEVVIRPAELPTQGTFSNVSTNVSPAPRPPTPAPTIASQPLSLRPAACRPAAGGAVHTRGLDFACDIYIWAPLAGTCGVLLLSLVITLYCNHRNRRRVCKCPRPVV

SEQ ID NO: 34 - protein, 22 aa, Artificial Sequence
  a.a. 252-273 of gp160
RPVVSTQLLLNGSLAEEEVVIR

SEQ ID NO: 35 - DNA, 492 bp, Artificial Sequence
  exemplary tag polynucleotide sequence
atgctgcttttggtaacttcactgcttctctgcgagctcccccaccccgcctttcttttgatcccttccgggggtggtagccgaccagtagtgagtactcagttgttgctcaatgggtcacttgctgaagaagaggtagtcattaggagtggaggtggcagcgaacttccgacccaaggcacattttcaaatgtgtctactaacgtctcaccagccaagcccactacgacatctgggggtggatcacctgcaccaaggccaccaactccggcgcccaccattgctagccaacccttgtctctgagacccgaagcctgccgccccgctgcgggaggcgcagtacatactaggggtctcgattttgcctgtgatatatatatctgggcacctcttgctggtacctgtggcgttcttttgttgtctttggtgataacattgtattgcaatcatcgcaatcgccgacgggtatgcaagtgtccacgacccgtcgtg

SEQ ID NO: 36 - protein, 164 aa, Artificial Sequence
  exemplary tag polypeptide sequence
MLLLVTSLLLCELPHPAFLLIPSGGGSRPVVSTQLLLNGSLAEEEVVIRSGGGSELPTQGTFSNVSTNVSPAKPTTTSGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 37 - DNA, 1857 bp, Artificial Sequence
  B2-TagBefore”, or tag is upstream of HSD11B2 (including overlap with vector) (1857bp)
atctcgaatcgccagcccgctgggccgccatgcttttgctggtgacttcccttctgctttgcgaactgcctcatccggcctttctcctgatcccccgcaggcccgtcgtgagcacccaactgttgctgaatggttcactggctgaggaggaggtcgtgatacggccagccgagcttcctacacaaggcacgttcagcaacgtcagcacgaatgtcagtcctgcgccacggcctcctaccccagcgccaaccatagcaagtcaaccgctcagcttgagaccagctgcatgcagacccgcagctggtggagctgtacatacacgaggccttgactttgcgtgtgatatttatatctgggcacccttggcagggacgtgtggcgtcctgctgctgtccctcgtaattacgctctactgcaaccacagaaaccgaaggagggtatgcaaatgtccacggcccgttgtcggcagcggtgaaggacgcggttcactcctcacgtgtggcgatgtggaagagaatccaggtccaggctctggggctactaacttcagccttcttaaacaggcgggagacgttgagaaccctggacctatggagcgttggccttggccatcgggtggtgcttggctgctcgtggctgctcgtgcactgctgcagctgctgcgttcagacctgcgtctgggtcgtccactgctggcagcactggcactgctggctgcactcgactggctgtgccagcgtctgctgcctccaccagctgcactcgctgtgctggctgctgctggttggatcgcattgtcccgtctggcacgtccacagcgtctgccagtggctactcgtgcagtgctcatcaccggttgtgactctggttttggtaaggagacggctaagaaactggactccatgggtttcacggtgctggctaccgtattggagttgaacagccctggtgctatcgagctgcgtacctgctgctcccctcgtctaaggctgctgcagatggacctgaccaaaccaggagacattagccgtgtgctagagttcaccaaggctcacaccaccagcaccggtctgtggggtctcgtcaacaacgcaggtcacaatgaagtagttgctgatgcagagctgtctccagtggctactttccgtagctgcatggaggtgaatttctttggtgcactcgagctgaccaagggtctcctgcctctgctgcgtagctcaaggggtcgtatcgtgactgtgggaagcccagcaggagacatgccatatccatgcttgggagcttatggaacctccaaagcagctgtggcactactcatggacacattcagctgtgaactccttccttggggagtcaaggtcagcatcatccagcctggttgcttcaagacagagtcagtgagaaacgtgggtcagtgggaaaagcgtaagcaattgctgctggctaacctgcctcaagagctgctgcaggcttacggtaaggactacatcgagcacttgcatggacagttcctgcactcgctacgtctggctatgtccgacctcaccccagttgtagatgctatcacagatgcactgctggcagctaggcctcgtcgtcgttattaccctggtcagggtctgggactcatgtacttcatccactactacctgcctgaaggtctgaggcgtcgtttcctgcaggctttcttcatcagtcactgtctgcctcgagcactgcagcctggtcagcctggtactaccccaccacaggacgcagctcaggacccaaacctgagccctggtccttccccagcagtggctaggtgacctgaaaaaggggtacctttaagaccaatgacttacaaggcagctgtag

SEQ ID NO: 38 - protein, 592 aa, Artificial Sequence
  translated “B2-TagBefore” (592 aa)
MLLLVTSLLLCELPHPAFLLIPRRPVVSTQLLLNGSLAEEEVVIRPAELPTQGTFSNVSTNVSPAPRPPTPAPTIASQPLSLRPAACRPAAGGAVHTRGLDFACDIYIWAPLAGTCGVLLLSLVITLYCNHRNRRRVCKCPRPVVGSGEGRGSLLTCGDVEENPGPGSGATNFSLLKQAGDVENPGPMERWPWPSGGAWLLVAARALLQLLRSDLRLGRPLLAALALLAALDWLCQRLLPPPAALAVLAAAGWIALSRLARPQRLPVATRAVLITGCDSGFGKETAKKLDSMGFTVLATVLELNSPGAIELRTCCSPRLRLLQMDLTKPGDISRVLEFTKAHTTSTGLWGLVNNAGHNEVVADAELSPVATFRSCMEVNFFGALELTKGLLPLLRSSRGRIVTVGSPAGDMPYPCLGAYGTSKAAVALLMDTFSCELLPWGVKVSIIQPGCFKTESVRNVGQWEKRKQLLLANLPQELLQAYGKDYIEHLHGQFLHSLRLAMSDLTPVVDAITDALLAARPRRRYYPGQGLGLMYFIHYYLPEGLRRRFLQAFFISHCLPRALQPGQPGTTPPQDAAQDPNLSPGPSPAVAR

SEQ ID NO: 39 - protein, 19 aa, Artificial Sequence
  P2A without GSG
ATNFSLLKQAGDVEENPGP

SEQ ID NO: 40 - protein, 22 aa, Artificial Sequence
  P2A with GSG
GSGATNFSLLKQAGDVEENPGP

SEQ ID NO: 41 - protein, 20 aa, Artificial Sequence
  E2A without GSG
QCTNYALLKLAGDVESNPGP

SEQ ID NO: 42 - protein, 23 aa, Artificial Sequence
  E2A with GSG
GSGQCTNYALLKLAGDVESNPGP

SEQ ID NO: 43 - protein, 22 aa, Artificial Sequence
  F2A without GSG
VKQTLNFDLLKLAGDVESNPGP

SEQ ID NO: 44 - protein, 25 aa, Artificial Sequence
  F2A with GSG
GSGVKQTLNFDLLKLAGDVESNPGP

SEQ ID NO: 45 - protein, 18 aa, Artificial Sequence
  T2A without GSG
EGRGSLLTCGDVEENPGP

SEQ ID NO: 46 - protein, 21 aa, Artificial Sequence
  T2A with GSG
GSGEGRGSLLTCGDVEENPGP